(19) United States
(12) Patent Application Publication
(10) Pub. No.: US 2024/0216611 A1
(43) Pub. Date: Jul. 4, 2024

(54) ON-BODY MEDICAMENT DELIVERY DEVICES FOR ADMINISTRATION OF MEDICAMENT

(71) Applicant: kaleo, Inc., Richmond, VA (US)

(72) Inventors: Paul F. MEYERS, Fishers, IN (US); Michael J. ROE, Powhatan, VA (US)

(73) Assignee: kaleo, Inc., Richmond, VA (US)

(21) Appl. No.: 18/288,965

(22) PCT Filed: May 9, 2022
(86) PCT No.: PCT/US2022/028334

§ 371 Date: Oct. 30, 2023

Related U.S. Application Data
(60) Provisional application No. 63/186,513, filed on May 10, 2021.

(51) Int. Cl. A61M 5/20; A61M 5/24

(52) U.S. Cl. CPC A61M 5/2046; A61M 5/2033; A61M 5/2448

(57) ABSTRACT

An apparatus includes a housing, a medicament container, a needle assembly, and an insertion member. The needle assembly includes a needle coupling member and a needle. A flow passageway is defined between a first end portion and a second end portion of the needle coupling member. The needle coupling member is rotatably coupled within the housing such that it rotates between a first orientation and a second orientation such that a portion of the needle extends from the housing in the second orientation. The insertion member is movable within the housing from a first position to a second position. A contact portion of the insertion member engages the second end portion of the needle coupling member to move the needle coupling member from the first orientation to the second orientation when the insertion member moves from the first position to the second position.

### CROSS-REFERENCE TO RELATED APPLICATIONS

This application claims benefit of priority to U.S. Provisional Application No. 63/186,513, entitled “On-Body Medicament Delivery Devices for Administration of Medicament,” filed May 10, 2021, which is incorporated herein by reference in its entirety.

### BACKGROUND

The embodiments described herein relate to medicament delivery devices. More particularly, the embodiments described herein relate to on-body medicament delivery devices for delivery of large volumes and/or viscous medicaments from a prefilled syringe or cartridge.

Advances in pharmaceutical products have resulted in new medicaments or therapeutic substances formulated to include high molecular weight compounds, compounds with complex molecular structures, living cells, and/or biologics. Such medicaments often have a very high viscosity (e.g., greater than about 100 centipoise at room temperature). As such, known delivery systems are often not able to produce the high forces needed to deliver these compositions. Additionally, some dosing regimens require high delivered volumes (e.g., 2 mL or more). Such increased dosages are often not practical with many known delivery systems, which may be designed for delivery of smaller volumes (e.g., 1.0 mL or 0.5 mL). Moreover, the increased delivery volumes may also result in longer delivery times (e.g., greater than 10 seconds), thus rendering certain delivery devices undesirable. In addition, many spring-based delivery systems that delivery larger volumes require longer springs that increase the overall size of the delivery device and therefore provide a more challenging and larger sized device for users to handle or carry.

As such, there has been increased interest in on-body delivery injectors, which can be temporarily attached to the patient's body during delivery. Such systems can allow for longer delivery times without requiring the patient or caregiver to hold the delivery device in place. Known on-body delivery systems, however, can be complicated to use, expensive, or incompatible with delivering high viscosity medicaments. For example, some known on-body delivery systems require that the user perform several operations to prepare the device for use (e.g., assembling portions of the device or transferring the desired dose from a vial into the device). Some known on-body delivery systems include an electronic system the produces the delivery force. Although possibly suitable for small molecule formulations (or formulations that are not highly viscous), such systems may not be able to generate the pressure required to deliver certain therapeutic substances. In addition, the complexity of some of these on-body delivery systems may lead to use-related hazards.

Other known systems include a spring-based actuation system that moves a piston rod to insert the needle or inject the medicament. Adapting such systems to deliver larger doses and/or more viscous substances, however, may not be practical due to the likely increase in the size (e.g., length) to accommodate the larger dose and produce the desired force. For example, the force and pressure necessary to overcome the resistance of a spring-based actuation system may be incompatible with the force and pressure required for the proper delivery of medicaments or therapeutic substances including high molecular weight compounds. Accordingly, many known delivery systems may not be able to provide appropriate force and/or develop the desired flow rate for effective delivery of such higher viscosity substances. Moreover, even if an injection device is capable of producing the desired force, such devices may result in undesirable delivery conditions or rates, which can compromise the substance being delivered or cause excessive pain or discomfort during the delivery process. For example, if the rate of delivery is too high, the resulting shear forces may damage the molecules within the substance, thereby reducing efficacy.

Other known systems may include a gas-based actuation system that relies on a quantity of pressurized gas to insert the needle and/or inject the medicament. However, the magnitude of the gas pressure required to insert the needle may be inconsistent with the magnitude of the gas pressure that is desirable to inject the medicament. At the same time, the magnitude of the gas pressure optimized for the injecting of the medicament may be inadequate to properly insert the needle. Accordingly, many known delivery systems may not be able to provide appropriate force and/or develop the desired flow rate for effective delivery of such higher viscosity substances. Moreover, even if an injection device can produce the desired force, such devices may result in undesirable delivery conditions or rates, which can compromise the substance being delivered or cause excessive pain or discomfort during the delivery process.

Additionally, when gas-based actuation systems are employed, it may be necessary to reduce the gas pressure within the injection device so that subsequent actions, such as needle retraction, may occur. However, a reduction in the gas pressure too quickly may result in an inadequate injection of the medicament. Similarly, the maintenance of the gas pressure at too great a magnitude following delivery of the medicament may result in the needle remaining inserted in the patient for a longer period than is required to inject the medicament.

Thus, a need exists for improved methods and devices for injection devices, including on-body delivery systems.

### SUMMARY

Medicament injectors, including on-body medicament delivery systems for administration of medicaments are described herein. In some embodiments, an apparatus includes a housing that defines a primary gas chamber and an insertion gas flow path. A medicament container is contained within the housing. The medicament container contains a medicament and includes an elastomeric member that seals the medicament within the medicament container, with the medicament container and the elastomeric member defining a medicament container gas chamber. The apparatus also includes a needle assembly. The needle assembly includes a needle carrier and a needle coupled to the needle carrier. The needle carrier defines a portion of a boundary of a needle actuation gas chamber. The needle carrier is configured to move within the housing between a first needle carrier position and a second needle carrier position, with the needle being within the housing when the needle carrier is in the first needle carrier position and outside of the housing when the needle is in the second needle carrier position. The apparatus includes a flow restriction assembly disposed with the housing. The flow restriction assembly defines a portion of a boundary of the primary gas chamber and a delivery gas flow path. The flow restriction assembly is configured to move within the housing to move the medicament container between a first container position and a second container position. The needle carrier is in fluid communication with the medicament container when the medicament container is in the second container position. Additionally, the apparatus includes an energy storage member configured to produce a pressurized gas when the energy storage member is actuated. The pressurized gas flows into the primary gas chamber to move the medicament container from the first container position to the second container position. A first portion of the pressurized gas flows within the insertion gas flow path and into the needle actuation gas chamber to move the needle carrier from the first needle carrier position to the second needle carrier position. A second portion of the pressurized gas flows through the delivery gas flow path and into the medicament container gas chamber to move the elastomeric member within the medicament container.

In some embodiments, an apparatus includes a housing that defines a primary gas chamber and an insertion gas flow path. The apparatus includes a medicament container within the housing. The medicament container contains a medicament and includes an elastomeric member that seals the medicament within the medicament container. The medicament container and the elastomeric member define a medicament container gas chamber. The apparatus includes a needle assembly having a needle carrier and a needle coupled to the needle carrier. The needle carrier is coupled to medicament container by a coupling member that selectively places the needle in fluid communication with the medicament container. The needle carrier defines a portion of a boundary of a needle actuation gas chamber. The needle carrier is configured to move within the housing been a first needle carrier position and a second needle carrier position, with the needle being within the housing when the needle carrier is in the first needle carrier position and outside of the housing when the needle is in the second needle carrier position. The apparatus also includes a flow restriction assembly disposed within the housing. The flow restriction assembly defines a portion of a boundary of the primary gas chamber and a delivery gas flow path. The flow restriction assembly is configured to move within the housing to move the medicament container between a first container position and a second container position. Additionally, the apparatus includes an energy storage member configured to produce a pressurized gas when the energy storage member is actuated. The pressurized gas flows into the primary gas chamber to move the medicament container from the first container position to the second container position. A first portion of the pressurized gas flows within the insertion gas flow path and into the needle actuation gas chamber to move the needle carrier from the first needle carrier position to the second needle carrier position. A second portion of the pressurized gas flows through the delivery gas flow path and into the medicament container gas chamber to move the elastomeric member within the medicament container.

In some embodiments, an apparatus includes a housing that defines a primary gas chamber and includes a vent portion. The apparatus includes a medicament container within the housing. The medicament container contains a medicament and includes an elastomeric member that seals the medicament within the medicament container. The medicament container and the elastomeric member define a medicament container gas chamber. Additionally, the elastomeric member is configured to move within the medicament container when a pressure within the medicament container gas chamber is greater than a first pressure threshold. The apparatus also includes a needle assembly that includes a needle carrier and a needle coupled to the needle carrier. The needle carrier defines a portion of a boundary of a needle actuation gas chamber, with the needle carrier being in fluid communication with the medicament container. The needle carrier is configured to move within the housing been a first needle carrier position and a second needle carrier position, with the needle being within the housing when the needle carrier is in the first needle carrier position and outside of the housing when the needle is in the second needle carrier position. Additionally, the apparatus includes an energy storage member configured to deliver a pressurized gas to the medicament container gas chamber and the needle actuation gas chamber via the primary gas chamber when the energy storage member is actuated. Further, the apparatus includes a vent assembly that includes a valve member within the vent portion of the housing. The vent portion defines an inlet orifice in fluid communication with the primary gas chamber and an outlet orifice in fluid communication with an exterior volume surrounding the housing. The valve member includes a seal positioned between the inlet orifice and the outlet orifice when the valve member is in a first valve position so as to fluidically isolate the primary gas chamber from the exterior volume. The valve member is configured to transition to a second valve position when the pressure within the medicament container gas chamber is greater than a second pressure threshold to place the primary gas chamber in fluid communication with the exterior volume via the inlet orifice and the outlet orifice. The second pressure threshold is greater than the first pressure threshold.

In some embodiments, an apparatus includes a housing, a medicament container, a needle assembly, and an insertion member. The medicament container is at least partially within the housing. The needle assembly includes a needle coupling member and a needle. The needle coupling member has a first end portion and a second end portion. A flow passageway is defined between the first end portion and the second end portion. The first end portion of the needle coupling member is coupled to the medicament container to place the flow passageway in fluid communication with the medicament container. The second end portion of the needle coupling member is coupled to the needle to place the needle in fluid communication with the flow passageway. The needle coupling member rotatably coupled within the housing such that it rotates between a first orientation and a second orientation. The needle is within the housing when the needle coupling member is in the first orientation, and a portion of the needle is outside of the housing when the needle coupling member is in the second orientation. The insertion member includes a contact portion. The insertion member is movable within the housing from a first position to a second position. The contact portion engages the second end portion of the needle coupling member to move the needle coupling member from the first orientation to the second orientation when the insertion member moves from the first position to the second position.

The apparatuses described herein may be employed to deliver a dose of a medicament in accordance with various described methods. For example, in some embodiments, the method may include placing a medical injector against the body. In this position, the medical injector is actuated such that an energy storage member produces a force within the primary gas chamber. The method may also include delivering a portion of the pressurized gas to the first medicament container gas chamber and the second medicament container gas chambers via the primary gas chamber. The first and second elastomeric members are maintained at a first longitudinal position until a pressure within the respective medicament container gas chamber is greater than the first pressure threshold. In accordance with the method, one of the elastomeric members is stopped at a second longitudinal position while the other elastomeric member is located between the first longitudinal position and the second longitudinal position. The pressure is maintained in the first and second medicament container gas chambers at a magnitude that is greater than the first pressure threshold and less than a second pressure threshold until each elastomeric member is positioned at the second longitudinal position. Following the positioning of each elastomeric member at the second longitudinal position, the method includes increasing the pressure within the first and second medicament container gas chambers. Additionally, the method includes transitioning the valve member to a second position when the pressure within at least one of the first medicament container gas chamber or second medicament gas chamber is greater than the second pressure threshold to place the primary gas chamber in fluid communication with the exterior volume.

### BRIEF DESCRIPTION OF THE DRAWINGS

FIG. 1 is a chart showing the time for delivery of 1 mL of a substance via a medicament delivery device according to an embodiment, as a function of the gas pressure and viscosity.

FIG. 2 is a front perspective view of a medicament delivery device according to an embodiment.

FIG. 3 is a rear perspective view of the medicament delivery device of FIG. 2.

FIG. 4 is atop view of the medicament delivery device of FIG. 2.

FIG. 5 is a side view of the medicament delivery device of FIG. 2.

FIG. 6 is a bottom perspective view of the medicament delivery device of FIG. 2 in a first needle configuration according to an embodiment.

FIG. 7 is a bottom perspective view of the medicament delivery device of FIG. 2 in a second needle configuration according to an embodiment.

FIG. 8 is a cross-sectional view of the medicament delivery device taken at line A-A in FIG. 5.

FIG. 9 is a cross-sectional view of the medicament delivery device taken at line B-B in FIG. 5.

FIG. 10 is a partial top view of a medicament delivery device in a first actuator configuration according to an embodiment.

FIG. 10A is an enlarged cross-sectional top view of a portion of the medicament delivery device of FIG. 10 showing the system actuator assembly.

FIG. 11 is a partial top view of a medicament delivery device in a second actuator configuration according to an embodiment.

FIG. 12 is a partial side perspective view of a medicament delivery device in a first needle configuration according to an embodiment.

FIG. 13 is a partial side perspective view of a medicament delivery device in a second needle configuration according to an embodiment.

FIG. 14 is a cross-sectional view of the medicament delivery device taken at line C-C in FIG. 5 in a first delivery configuration.

FIG. 14A is an enlarged view of the medicament delivery device of FIG. 14 showing the gas vent assembly and the flow control mechanism.

FIG. 15 is a cross-sectional view of the medicament delivery device taken at line C-C in FIG. 5 in a second delivery configuration.

FIG. 16 is a cross-sectional view of the medicament delivery device taken at line C-C in FIG. 5 in a third delivery configuration.

FIG. 17 is a cross-sectional view of the medicament delivery device taken at line C-C in FIG. 5 in a fourth delivery configuration.

FIG. 18 is a cross-sectional view of the medicament delivery device taken at line D-D in FIG. 4 in a first needle orientation.

FIG. 19 is a cross-sectional view of the medicament delivery device taken at line D-D in FIG. 4 in a second needle orientation.

FIG. 20 is a perspective view of an insertion member according to an embodiment.

FIG. 21 is a bottom perspective view of a needle assembly according to an embodiment.

FIG. 22 is a top perspective view of the needle assembly of FIG. 21.

FIG. 23 is a cross-sectional view of the needle assembly of FIG. 22.

FIG. 24 is a front perspective view of a medicament delivery device according to an embodiment.

FIG. 25 is a side perspective view of the medicament delivery device of FIG. 24.

FIG. 26 is a side view of the medicament delivery device of FIG. 24.

FIG. 27 is a partial top perspective view of a medicament delivery device in a second actuator configuration according to an embodiment.

FIG. 28 is a cross-sectional view of the medicament delivery device taken at line E-E in FIG. 24 in a first delivery configuration.

FIG. 29 is a cross-sectional view of the medicament delivery device taken at line E-E in FIG. 24 in a second delivery configuration.

FIG. 30 is a cross-sectional view of the medicament delivery device taken at line E-E in FIG. 24 in a third delivery configuration.

FIG. 30A is an enlarged view of the delivery control assembly in FIG. 29.

FIG. 31 is a cross-sectional view of the medicament delivery device taken at line E-E in FIG. 24 in a fourth delivery configuration.

FIG. 32 is a cross-sectional view of the medicament delivery device taken at line E-E in FIG. 24 in a fifth delivery configuration.

FIG. 32A is an enlarged view of the delivery control assembly in FIG. 31.

FIG. 33 is a cross-sectional view of the medicament delivery device taken at line E-E in FIG. 24 in a sixth delivery configuration.

FIG. 34 is a front perspective view of a medicament delivery device according to an embodiment.

FIG. 35 is a side perspective view of the medicament delivery device of FIG. 24.

FIG. 36 is a side view of the medicament delivery device of FIG. 34.

FIG. 37 is a cross-sectional view of the medicament delivery device taken at line F-F in FIG. 36 in a first delivery configuration.

FIG. 38 is a cross-sectional view of the medicament delivery device taken at line F-F in FIG. 36 in a second delivery configuration.

FIG. 39 is a cross-sectional view of the medicament delivery device taken at line F-F in FIG. 36 in a third delivery configuration.

FIG. 40 is a cross-sectional view of the medicament delivery device taken at line F-F in FIG. 36 in a fourth delivery configuration.

FIG. 41 is a cross-sectional view of the medicament delivery device taken at line F-F in FIG. 36 in a fifth delivery configuration.

FIG. 41A is an enlarged cross-sectional view of the delivery control assembly in FIG. 41.

FIG. 42 is a cross-sectional view of the medicament delivery device taken at line F-F in FIG. 36 in a sixth delivery configuration.

FIG. 42A is an enlarged cross-sectional view of the delivery control assembly in FIG. 42.

FIG. 43 is a top perspective view of a needle assembly of FIG. 37.

FIG. 44 is a cross-sectional view of the needle assembly of FIG. 43.

FIG. 45 is a top perspective view of a medicament delivery device according to an embodiment.

FIG. 46 is a top view of the medicament delivery device of FIG. 45.

FIG. 47 is a bottom perspective view of the medicament delivery device of FIG. 45.

FIG. 48 is a schematic view of a medicament delivery device in a first configuration according to an embodiment.

FIG. 49 is a schematic view of the medicament delivery device of FIG. 48 in a second configuration.

FIG. 50 is a front perspective view of a medicament delivery device according to an embodiment.

FIG. 51 is a bottom perspective view of the medicament delivery device of FIG. 50 in a first configuration.

FIG. 52 is a top view of the medicament delivery device of FIG. 50.

FIG. 53 is a side view of the medicament delivery device of FIG. 50.

FIG. 54 is an end view of the medicament delivery device of FIG. 50 with an outer cover removed.

FIG. 55 is a side perspective view of the medicament delivery device of FIG. 50 in a second configuration.

FIG. 56 is a top perspective view of the medicament delivery device of FIG. 50 with an outer cover removed.

FIG. 57 is a top perspective view of the medicament delivery device of FIG. 56 with a portion of the housing removed.

FIG. 58 is a partial top perspective view of the medicament delivery device of FIG. 56 in the first configuration with a portion of the housing removed.

FIG. 59 is a side view of the medicament delivery device of FIG. 56 in the first configuration with a portion of the housing removed.

FIG. 60 is a partial perspective cross-sectional view of the medicament delivery device of FIG. 56, taken along the line X₁-X₁ in FIG. 53, with the medicament container in a first container position.

FIG. 61 is a partial perspective cross-sectional view of the medicament delivery device of FIG. 55, taken along the line X₁-X₁ in FIG. 53, with the medicament container in a second container position.

FIG. 62 is a side cross-sectional view of the medicament delivery device of FIG. 56, taken along the line X₂-X₂ in FIG. 52, in a first configuration.

FIG. 63 is a side cross-sectional view of the medicament delivery device of FIG. 55, taken along the line X₂-X₂ in FIG. 52, in a second configuration.

FIG. 64 is an end perspective view of a flow restriction assembly according to an embodiment.

FIG. 65 is a cross-sectional view of the medicament delivery device of FIG. 55, taken along the line X₁-X₁ in FIG. 53, with an expandable assembly in a second configuration.

FIG. 66 is an enlarged cross-sectional view of a portion of a vent assembly of the medicament delivery device of FIG. 65.

FIG. 67 is a cross-sectional view of the medicament delivery device of FIG. 55, taken along the line X₁-X₁ in FIG. 53, with the elastomeric members at a second longitudinal position.

FIG. 68 is an enlarged cross-sectional view of a portion of a vent assembly of the medicament delivery device of FIG. 66 illustrating the valve member in a second position.

FIG. 69 is a flow chart of a method for delivering a medicament according to an embodiment.

### DETAILED DESCRIPTION

Medical injectors including on-body medicament delivery devices for administration of medicaments are described herein. In some embodiments, an apparatus includes a housing, a medicament container/medicament container assembly, a needle assembly, and an insertion member. The medicament container is at least partially within the housing. The needle assembly includes a needle coupling member and a needle. The needle coupling member has a first end portion and a second end portion. A flow passageway is defined between the first end portion and the second end portion. The first end portion of the needle coupling member is coupled to the medicament container to place the flow passageway in fluid communication with the medicament container. The second end portion of the needle coupling member is coupled to the needle to place the needle in fluid communication with the flow passageway. The needle coupling member rotatably coupled within the housing such that it rotates between a first orientation and a second orientation. The needle is within the housing when the needle coupling member is in the first orientation, and a portion of the needle is outside of the housing when the needle coupling member is in the second orientation. The insertion member includes a contact portion. The insertion member is movable within the housing from a first position to a second position. The contact portion engages the second end portion of the needle coupling member to move the needle coupling member from the first orientation to the second orientation when the insertion member moves from the first position to the second position.

As used herein, the terms “substance” or “medicament” includes any constituent of a therapeutic substance. A medicament can include such constituents regardless of their state of matter (e.g., solid, liquid or gas). Moreover, a medicament can include the multiple constituents that can be included in a therapeutic substance in a mixed state, in an unmixed state and/or in a partially mixed state. A medicament can include both the active constituents and inert constituents of a therapeutic substance. Accordingly, as used herein, a medicament can include non-active constituents such as, water, colorant or the like.

The term “about” when used in connection with a referenced numeric indication means the referenced numeric indication plus or minus up to 10 percent of that referenced numeric indication. For example, “about 100” means from 90 to 110.

In a similar manner, term “substantially” when used in connection with, for example, a geometric relationship, a numerical value, and/or a range is intended to convey that the geometric relationship (or the structures described thereby), the number, and/or the range so defined is nominally the recited geometric relationship, number, and/or range. For example, two structures described herein as being “substantially parallel” is intended to convey that, although a parallel geometric relationship is desirable, some non-parallelism can occur in a “substantially parallel” arrangement. By way of another example, a structure defining a volume that is “substantially 0.50 milliliters (mL)” is intended to convey that, while the recited volume is desirable, some tolerances can occur when the volume is “substantially” the recited volume (e.g., 0.50 mL). Such tolerances can result from manufacturing tolerances, measurement tolerances, and/or other practical considerations (such as, for example, minute imperfections, age of a structure so defined, a pressure or a force exerted within a system, and/or the like). As described above, a suitable tolerance can be, for example, of 10% of the stated geometric construction, numerical value, and/or range. Furthermore, although a numerical value modified by the term “substantially” can allow for and/or otherwise encompass a tolerance of the stated numerical value, it is not intended to exclude the exact numerical value stated.

As used herein, the term “set” can refer to multiple features or a singular feature with multiple parts. For example, when referring to set of walls, the set of walls can be considered as one wall with multiple portions, or the set of walls can be considered as multiple, distinct walls. Thus, a monolithically-constructed item can include a set of walls. Such a set of walls can include, for example, multiple portions that are either continuous or discontinuous from each other. A set of walls can also be fabricated from multiple items that are produced separately and are later joined together (e.g., via a weld, an adhesive, or any suitable method).

As used in this specification and the appended claims, the words “proximal” and “distal” refer to direction closer to and away from, respectively, an operator of the medical device. Thus, for example, the end of the medicament delivery device contacting the patient's body would be the distal end of the medicament delivery device, while the end opposite the distal end would be the proximal end of the medicament delivery device.

As used herein, the terms “stiffness” or “rigidity” relate to an object's resistance to deflection, deformation, and/or displacement produced by an applied force, and is generally understood to be the opposite of the object's “flexibility.” For example, a gas release member with greater stiffness is more resistant to deflection, deformation and/or displacement when exposed to a force than a gas release member having a lower stiffness. Similarly stated, a gas release member having a higher stiffness can be characterized as being more rigid than a gas release member having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of a portion of the object different than the portion of the object to which the force is directly applied. Said another way, in some objects, the point of deflection is distinct from the point where force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness (or flexibility) of the object can be decreased by decreasing and/or changing the shape of the object.

Thus, an object that deforms readily under small forces, such as, for example, a wire, a filament, a cord, or the like is said to be a flexible object.

The therapeutic compositions described herein can be included in any suitable medicament delivery device as described herein or in International Patent Publication No. WO2017/004345, entitled “Auto-Injectors for Administration of a Medicament Within a Prefilled Syringe,” filed Jun. 30, 2016 (“the ′4345 PCT”), International Patent Publication No. WO2020/140040, entitled “Devices and Methods for Delivery of Substances Within a Prefilled Syringe,” filed Dec. 27, 2019 (“the ′0040 PCT”), International Patent Publication No. WO2018/136413, entitled “Medicament Delivery Devices with Wireless Connectivity and Event Detection,” filed Jan. 16, 2018 (“the ′6413 PCT”), and/or WO2020/018433, entitled “Medicament Delivery Devices with Wireless Connectivity and Compliance Detection,” filed Jul. 15, 2019 (“the ′8433 PCT”), each of which is incorporated herein by reference in its entirety. For example, in some embodiments, a drug product configured for administration by an untrained user (such a person accompanying the patient) can include a dose of icatibant. Such drug products can include, for example, an auto-injector having a needle length and delivery profile (e.g., flow of the icatibant) sufficient to produce subcutaneous injection. In other embodiments, a drug product can include a therapeutic substance including of a monoclonal antibody. Such drug products can include, for example, an auto-injector having multiple prefilled syringe containers and that delivers the medicament from each of the syringes in one operation to deliver the desired dose. By including multiple syringes, such arrangements can allow for higher doses while still using a standard fill volume within the prefilled syringe.

In some embodiments, a gas-powered medicament delivery device can result in a compact device, in which the outer dimensions of the housing are not substantially larger than the length of the medicament container disposed therein. For example, as shown and described herein, in some embodiments, a medicament delivery device can be devoid of a mechanical linkage that exerts or transfers a force to an elastomeric member to expel a medicament from a medicament container therein. Similarly stated, in some embodiments, a medicament delivery device can be devoid of mechanical linkages (rams, rods) that transfer force to the elastomeric member. Rather, in some embodiments, the elastomeric member can exert a force onto a member (e.g., an expandable member) to provide control over the delivery. Such medicament delivery devices (or medicament delivery mechanisms) are considered to be “pistonless” systems. As one example, in a pistonless, gas-powered auto-injector, the force exerted by the gas can move the medicament container relative to the housing and similarly, can move the elastomeric member relative to (e.g., within) the medicament container. In some embodiments, by not including a movable mechanism, a piston, and/or the like, a height of the medical injector can be reduced relative to, for example, the height of a device that includes a rigid, single length piston.

For example, any of the medicament delivery devices described herein can include any suitable “pistonless” design, such as those described in the ′4345 PCT, the ′0040 PCT, or in International Patent Publication No. WO 2016/154427, entitled “DEVICES AND METHODS FOR DELIVERING A LYOPHILIZED MEDICAMENT,” filed on Mar. 24, 2016, which is incorporated herein by reference in its entirety.

In some embodiments, the characteristics of the medicament, the medicament container and the needle are such that the force required to achieve the desired injection is not possible via manual injection. Accordingly, in some embodiments a device can include an energy storage member configured to produce the desired force (and/or pressure within the medicament container) to deliver the medicament. For example, in certain circumstances, the pressure of the medicament within a needle-based medicament container can be modeled by the Hagen-Poiseuille law, as indicated below:

$$P = ( 8 * μ * L * Q ) / ( Π * {R}^{4} ) \qquad ( 1 )$$

where P is the pressure of the medicament within the medicament container, is the viscosity of the medicament, L is the length of the needle (not shown), Q is the flow rate of the medicament through the needle, and R is the radius of the lumen defined by the needle. Because the pressure (and/or force) required to inject a high viscosity fluid through a small-bore needle is proportional to the inverse of the radius of the lumen of the needle to the fourth power, the pressure of the medicament within the medicament container necessary to achieve the desired flow rate can, at times, be relatively high. By including a gas-based energy storage member, the desired pressure can be achieved.

In some embodiments, the energy storage member can be configurable to include various amounts of stored energy without changing the size of the energy storage member. In such embodiments, therefore, a high force (e.g., to inject viscous medicaments) can be achieved in the same packaging that is used for lower viscosity medicaments. For example, in some embodiments, the energy storage member can be a compressed gas cylinder having any desired pressure (and thus, mass) of gas therein. Accordingly, the pressure and/or force can be achieved to complete the operations described herein, regardless of the medicament.

In such embodiments, the use of a non-mechanical energy storage member (e.g., gas, propellant, magnetic, electronic or the like) can produce a sufficiently high force to produce the desired pressure within the medicament container to produce the desired injection. For example, in such embodiments having a larger diameter, the amount of force needed to produce a desired internal pressure increases significantly. In some embodiments, any of the medicament delivery devices shown herein can include a gas-based energy storage system configured to produce a gas pressure (e.g., within the gas chamber) of between about 200 psi and about 2700 psi. In some embodiments, any of the injectors shown herein can include a gas-based energy storage system configured to produce a gas pressure of about 200 psi, 300 psi, 400 psi, 500 psi, 600 psi, 700 psi, 800 psi, 900 psi, 1100 psi, 1200 psi, 1300 psi, 1500 psi, 1700 psi, 1900 psi, 2100 psi, 2300 psi, 2500 psi, or 2700 psi. In some embodiments, any of the injectors shown herein can include a gas-based energy storage system configured to produce a gas pressure of between about 200 psi to 7000 psi. The gas pressure can be produced by any suitable mechanism, such as, for example, by puncturing a compressed gas container, releasing a propellant (e.g., hydrofluoroalkane), releasing a refrigerant (e.g., R134a), releasing a liquefied gas, triggering a chemical reaction, or the like.

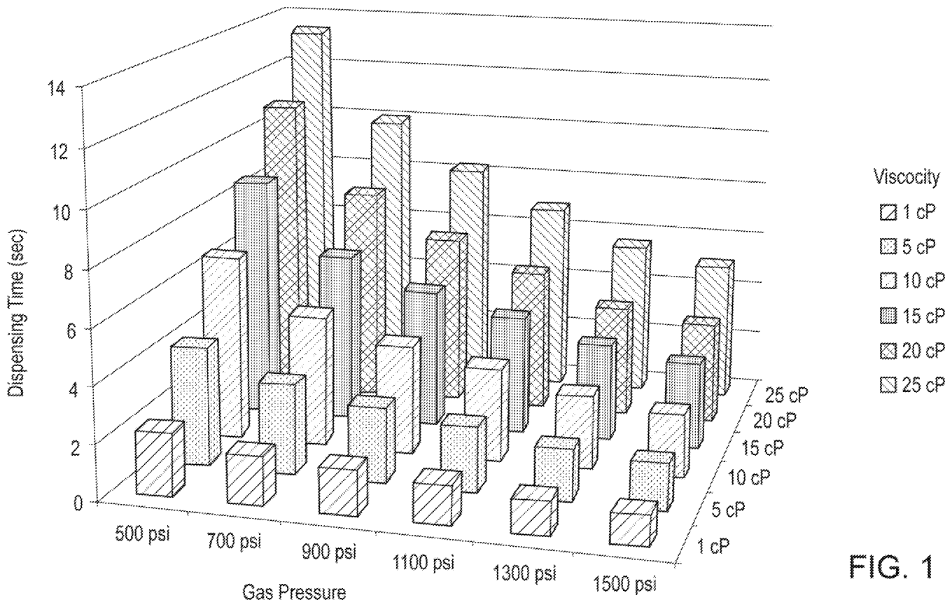

FIG. 1 is a chart showing the delivery time for delivering 1 mL of a substance using a medicament delivery device of the types shown and described herein and in the ′4335 PCT and the ′0040 PCT as a function of both the gas pressure and the viscosity of the substance. As shown, the delivery time can be tailored to meet desired performance characteristics by adjusting the gas pressure within the device. For example, in some embodiments, any of the devices and drug products described herein can be used to perform a method of subcutaneous injection by limiting the gas pressure during the injection event. By limiting the gas pressure, the injection force (and therefore the momentum of the substance leaving the device) can be reduced to ensure that the substance is delivered subcutaneously and not intramuscularly. Control of the momentum of the substance leaving the device and the injection speed of the substance can also minimize pain to a patient, particularly when the substance is highly viscous (e.g., greater than about 100 centipoise at room temperature). By way of another example, in some embodiments, injection of medicaments or therapeutic substances including high molecule weight compounds (e.g., greater than about 5 kDa) may require an injection force less than a force required to overcome a spring-based actuation system of an autoinjector to prevent shearing and therefore damage to the medicament or therapeutic substance.

In some embodiments, the gas pressure can be controlled during the injection event by limiting the amount of pressurized gas within the compressed gas container. In other embodiments, the gas pressure can be controlled by selective movement of a gas release valve, such as the release valve described below, during a delivery (e.g., injection) event. Similarly stated, in some embodiments, any of the devices described herein (or in the ′4345 PCT, the ′0040 PCT, or the ′6413 PCT) can include a valve and a mechanism that opens the valve by a predetermined amount during a delivery event. In yet other embodiments, any of the devices described herein (or in the ′4345 PCT, the ′0040 PCT, or the ′6413 PCT) can include a porous flow restriction member that provides some amount of pressure reduction during a delivery event.

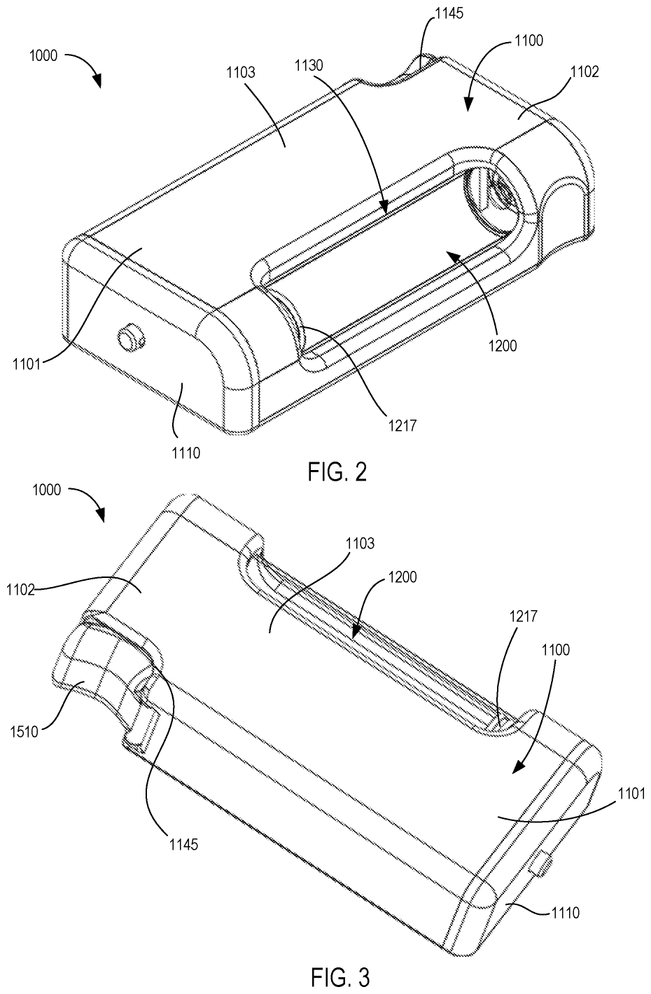

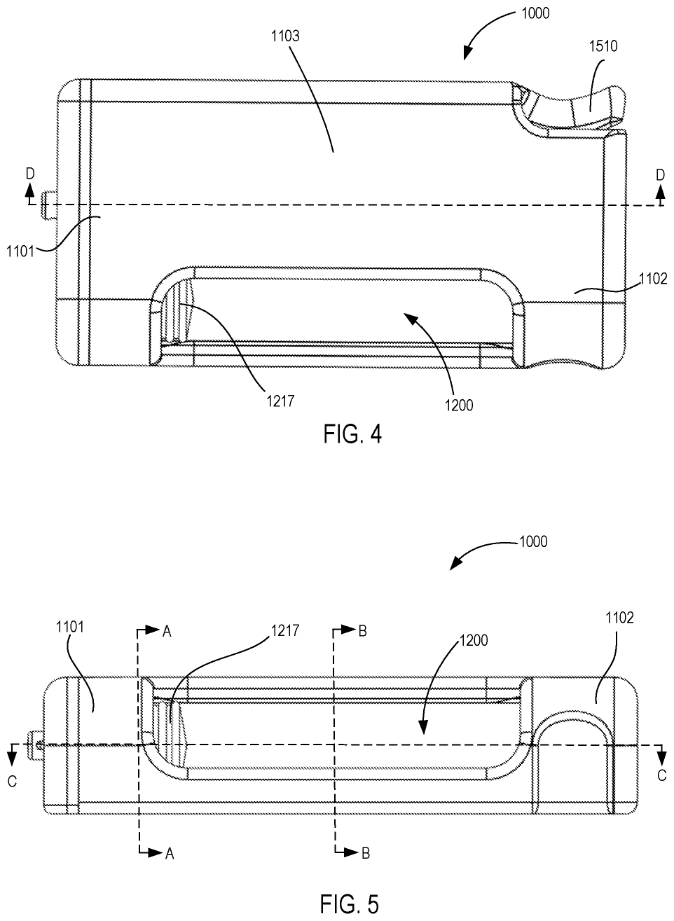

In some embodiments, a medicament delivery device can be an auto-injector having a pistonless delivery system in which the force exerted by the gas can move a needle assembly to extend at least partially from the auto-injector and move an elastomeric member relative to (e.g., within) the medicament container assembly. For example, FIGS. 2-19 show a medicament delivery device 1000 (also referred to herein as “auto-injector,” “injector,” “medical injector,” or “device”), according to an embodiment. The medicament delivery device 1000 is a gas-powered auto-injector configured to deliver a medicament contained within a medicament container assembly 1200, as described herein. In some embodiments, the medicament container assembly is a pre-filled syringe or pre-filled cartridge. In other embodiments, the medicament container assembly can be a syringe or cartridge than can be filled by a user or a healthcare professional. The housing can include a lid or end cap that can be secured to the housing via a latch or other locking mechanism so that the user can load the pre-filled syringe of pre-filled cartridge into the medical injector prior to use. The lid can include a sealing mechanism such as an O-ring in order to ensure a tight seal onto the housing to ensure no gas escapes during activation and subsequent delivery of the medicament. A discussion of the components of the medical injector 1000 will be followed by a discussion of the operation of the medical injector 1000. Certain aspects of the medical injector 1000 can be similar to or substantially the same to the medical injectors described in the ′4345 PCT, the ′0040 PCT, the ′6413 PCT, U.S. patent application Ser. No. 13/357,935 (now U.S. Pat. No. 9,084,849) entitled, “MEDICAMENT DELIVERY DEVICES FOR ADMINISTRATION OF A MEDICAMENT WITHIN A PREFILLED SYRINGE,” filed on Jan. 25, 2012 (referred to henceforth as the “′849 patent”), the disclosures of each of which are incorporated herein by reference in its entirety.

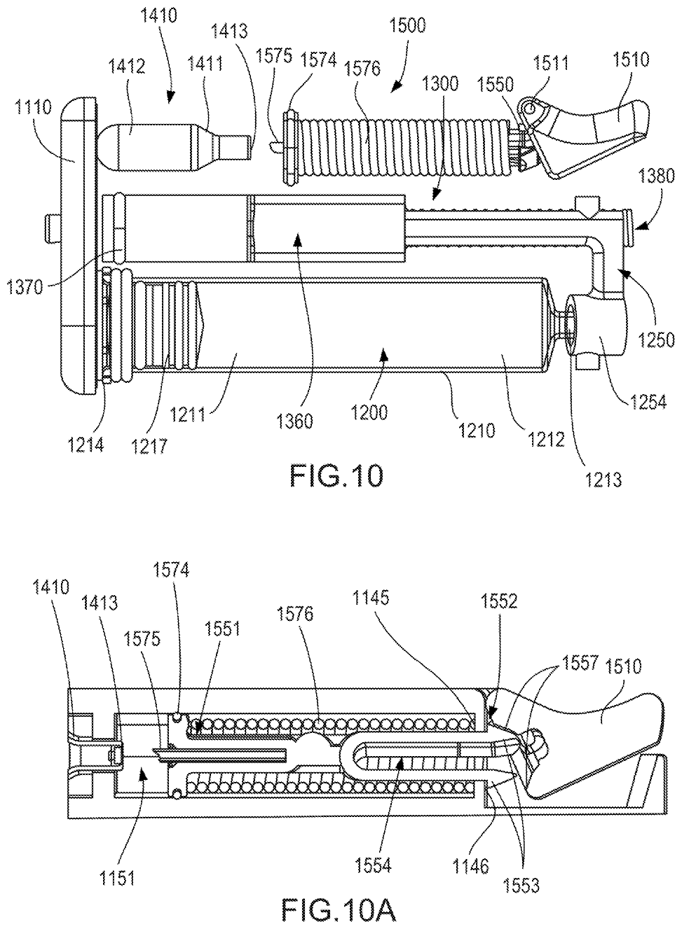

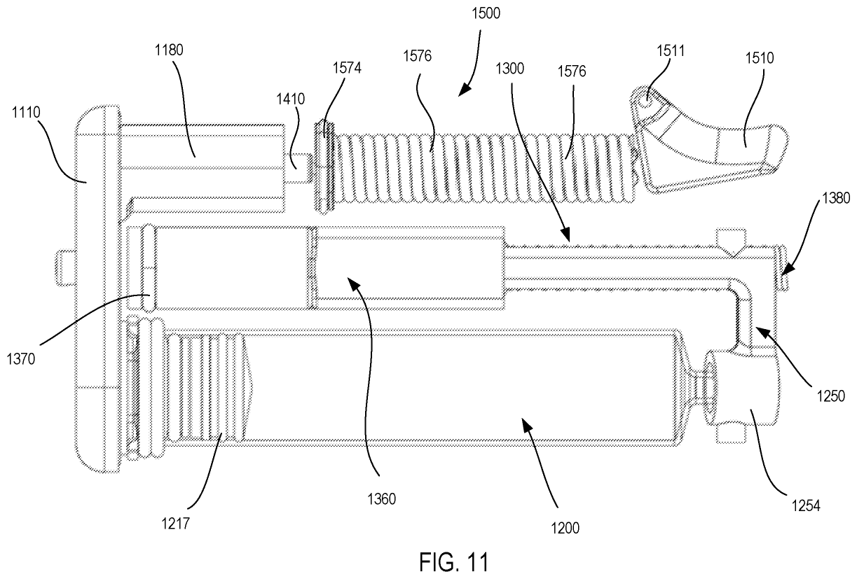

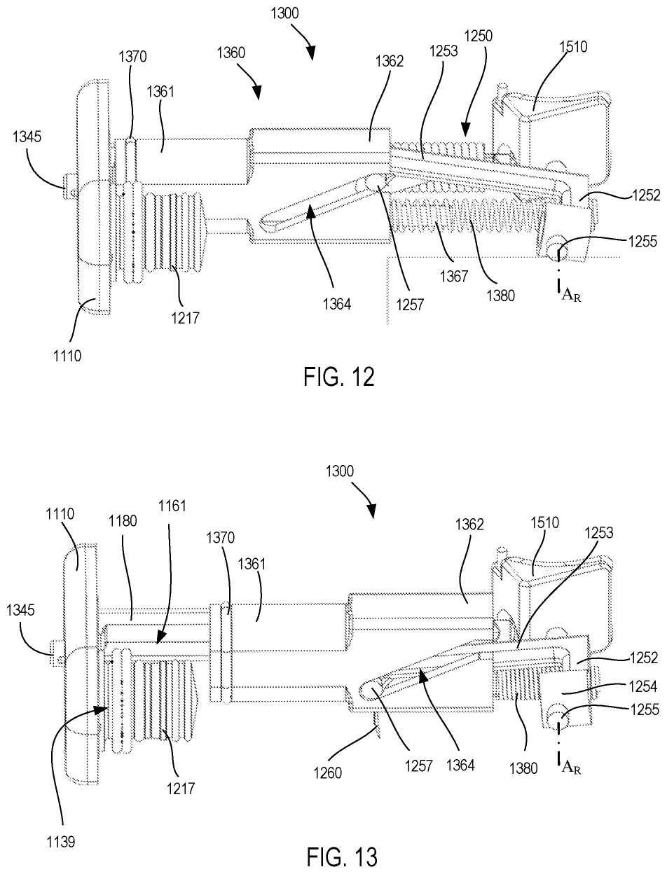

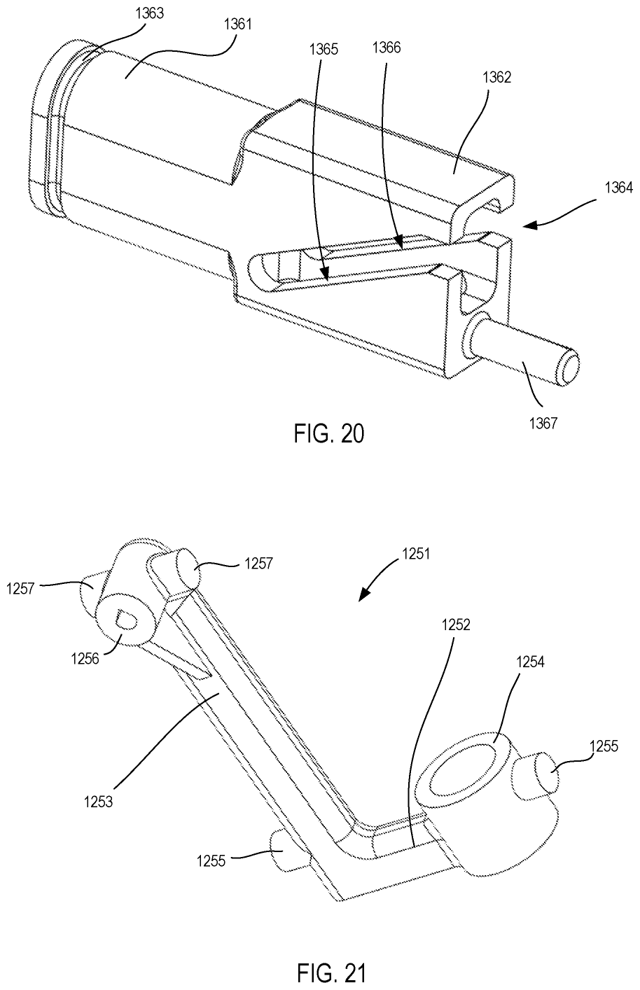

FIGS. 2-23 show a medicament delivery device 1000 (also referred to as a medical injector or on body delivery device) according to an embodiment. The medicament delivery device 1000 includes a housing 1100 (see e.g., FIGS. 2-7), a system actuation assembly 1500 (see e.g., FIGS. 10 and 11), a medicament container assembly 1200 (see FIGS. 10-11 and 14-17), a medicament delivery mechanism 1300 (see e.g., FIGS. 12-19). As shown in FIGS. 2-7, the housing 1100 has a first end portion 1101 and a second end portion 1102. The first end portion 1101 is the end of the device where pressurize gas is vented, and is opposite the end where the medicament exits from the medicament container assembly 1200; the first end portion 1101 is also referred to herein as the proximal end portion. The second end portion 1102 is the end of the device where the delivery end of the medicament container assembly 1200is located and is opposite the end where the gas chamber 1440 is defined; the second end portion is also referred to herein as the distal end portion. The housing 1100 includes a top portion 1103 extending between the first end portion 1101 and a second end portion 1102. The top portion 1103 defines a status indicator aperture 1130. The status indicator aperture 1130 can allow a patient to monitor the status and/or contents of the medicament container assembly 1200, a position of an elastomeric member 1217 within the medicament container assembly 1200, and/or and the medicament contained within the housing 1100. For example, by visually inspecting the status indicator apertures 1130, a patient can determine whether the medicament container assembly 1200 contains a medicament and/or whether the medicament has been dispensed. In some embodiments, the status indicator aperture 1130 includes a length to display an entire, or substantially the entire, stroke length of the elastomeric member 1217 during operation.

The housing 1100 includes a bottom portion 1104 extending between the first end portion 1101 and a second end portion 1102. The bottom portion 1104 includes a contact surface for contacting a body surface of a patient. The bottom portion 1104 includes a needle aperture 1105 configured to allow a needle 1260 of the needle assembly 1250 to pass through during operation. In some embodiments, the bottom portion 1104 includes an adhesive material for temporarily securing the bottom portion 1104 to the body surface of the patient. In some embodiments, the bottom portion 1104 can be covered by a removable film, which can function to protect the adhesive material and also cover the needle aperture 1105.

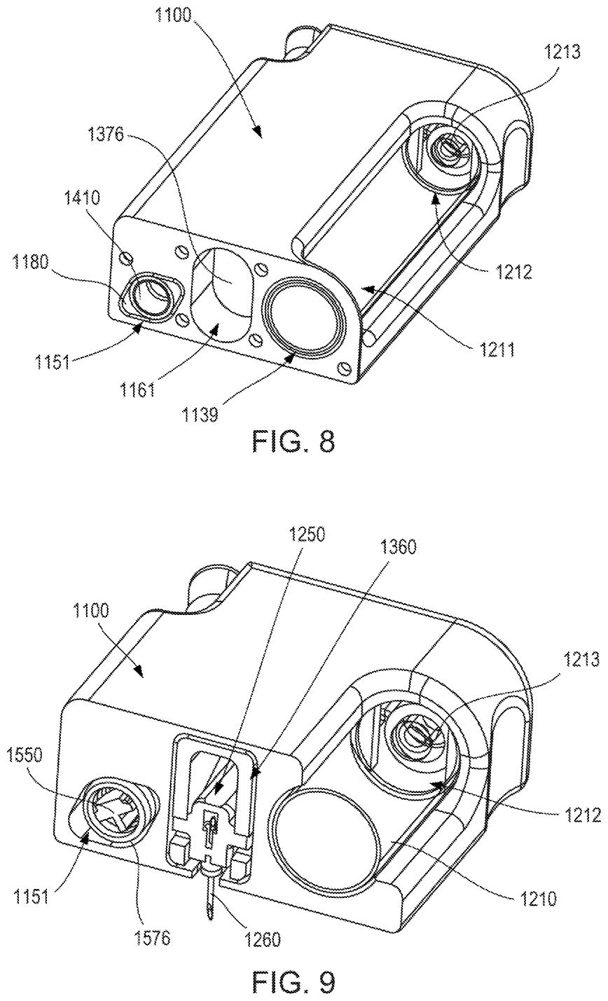

As shown in FIGS. 8, 9, and 14-17, the housing 1100 defines a medicament cavity 1139, a gas container cavity 1151, and a delivery mechanism cavity 1161. The gas container cavity 1151 is configured to receive the gas container 1410 and a portion of the system actuator assembly 1500 (e.g., a release member 1550 and the spring 1576, as shown in FIGS. 8-11). The first (i.e., proximal) end portion of the gas container cavity 1151 is configured to support the gas container retention member 1180, as described in further detail herein. The gas container cavity 1151 is in fluid communication with the medicament cavity 1139 and the delivery mechanism cavity 1161 via a gas passageway 1135 defined in the housing 1100, as described in further detail herein.

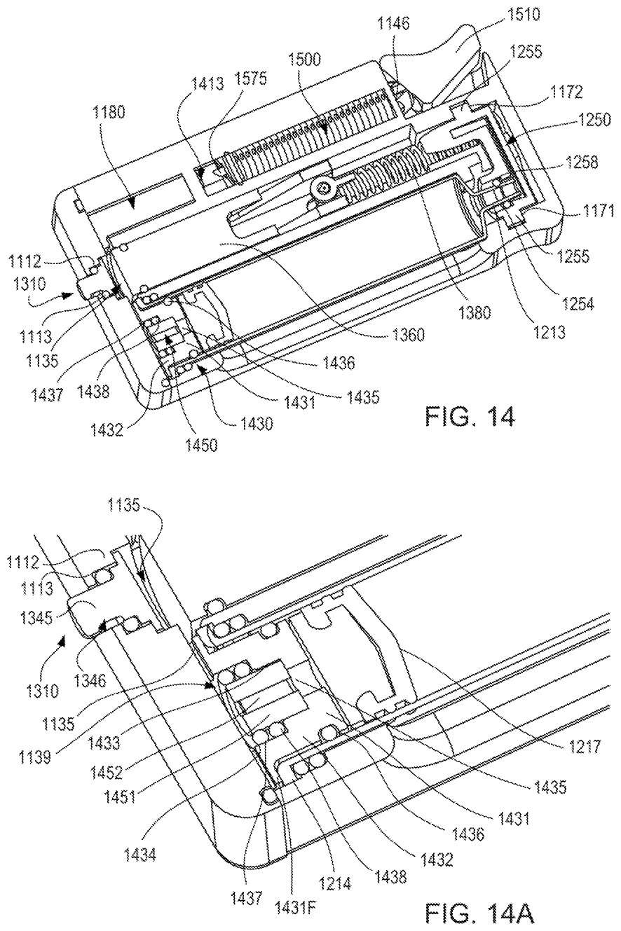

The medicament cavity 1139 is configured to receive the medicament container assembly 1200 and at least a portion of the medicament delivery mechanism 1300. In particular, as described below, the medicament delivery mechanism 1300 includes an insertion member 1360 (also referred to as a needle assembly carrier) and a needle assembly 1250 (see e.g., FIGS. 12, 13, 18 and 19). The medicament cavity 1139 is in fluid communication with the gas container cavity 1151, the delivery mechanism cavity 1161, and a vent opening 1112 (see e.g., FIGS. 14A, 16, and 17).

The first (i.e., proximal) end portion 1101 of the housing 1100 includes a housing cap 1110 (see e.g., FIGS. 8-11). A gas container retention member 1180 is configured to be inserted into the gas container cavity 1151 to retain a gas container 1410 that contains a pressurized gas (see e.g., FIGS. 10 and 11). The housing cap 1110 is configured to retain the gas container retention member 1180 within the gas container cavity 1151. When the medicament delivery device 1000 is actuated, pressurized gas from the gas container 1410 is conveyed from the gas container cavity 1151 to the medicament cavity 1139 and to the delivery mechanism cavity 1161 via the gas passageway 1135 of the housing 1100. Said another way, the gas passageway 1135 places the gas container cavity 1151 in fluid communication with the medicament cavity 1139 and the delivery mechanism cavity 1161. Thus, the pressurized gas from the gas container 1410 is conveyed from the gas container cavity 1151 to the delivery mechanism cavity 1161 to move the insertion member 1360 and the needle assembly 1250. The pressurized gas conveyed to the proximal portion of the medicament cavity 1139 also serves as a pressurized gas reservoir used to inject the medicament, as described herein.

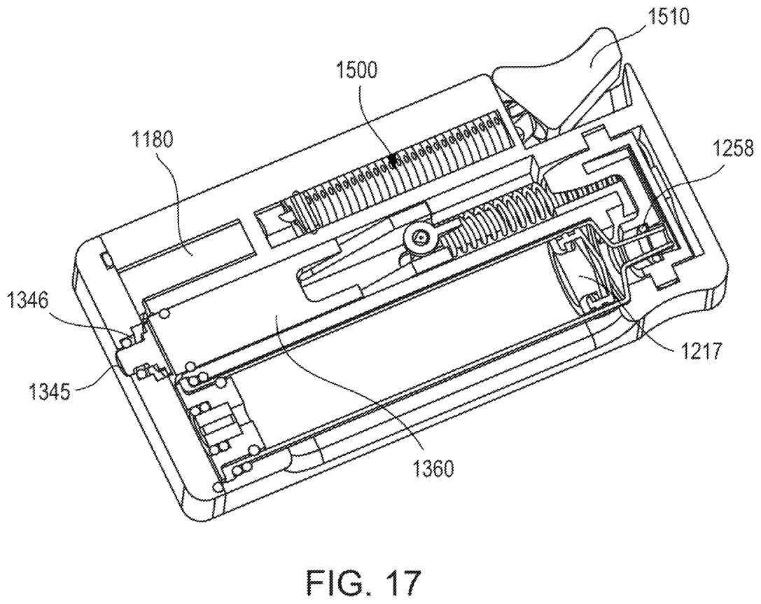

The housing cap 1110 includes an O-ring 1113 and defines the vent opening 1112. The vent opening 1112 provides a passageway through which pressurized gas is conveyed from the medicament cavity 1139 and from the delivery mechanism cavity to a volume outside of the medicament delivery device 1000. In this manner, the force produced by the pressurized gas on the insertion member 1360 can be reduced to allow needle retraction after the injection is completed. As shown in FIG. 17, the O-ring 1113, in conjunction with the valve portion 1345 of the gas vent assembly 1310, selectively seals the vent opening 1112 during needle insertion and delivery of the medicament.

Although the vent opening 1112 is shown as being defined by the housing cap 1110, and being in a proximal surface thereof, in other embodiments, the vent opening 1112 (and any of the vent openings described herein) can be defined within any suitable portion of the housing cap or side wall of the housing 1100. For example, in some embodiments, the vent opening 1112 (and any of the vent openings described herein) can be defined by the housing cap 1110, but can have a centerline that is nonparallel to a longitudinal axis of the medicament delivery device 1000. Said another way, in some embodiments, the vent opening 1112 (and any of the vent openings described herein) can open towards a side of the medical injector, rather than opening towards the proximal end, as shown. In other embodiments, the vent opening 1112(and any of the vent openings described herein) can be defined by any wall and/or surface of the housing 1100. In some embodiments, the vent opening 1112 is provided on the bottom portion 1104. In some embodiments, the valve portion 1345 protrudes from the bottom portion 1104 and is configured to selectively place the vent opening 1112 in an open position (i.e., place the gas passageway 1135 in fluid communication with an external environment) when lift-off of the medicament delivery device is detected. For example, the valve portion 1345 is configured to move from a first position (i.e., valve portion 1345 depressed into the housing 1100 due to contact by the patient's body) to a second position (i.e., valve portion expands out of housing 1100 due to bottom portion 1104 being moved away from the patient's body). In some embodiments, the valve portion 1345 is coupled to a spring such that the valve portion 1345 protrudes from the housing 1100 and contact against the patient's body works against the force of the spring to depress the valve portion 1345 into the housing 1100.

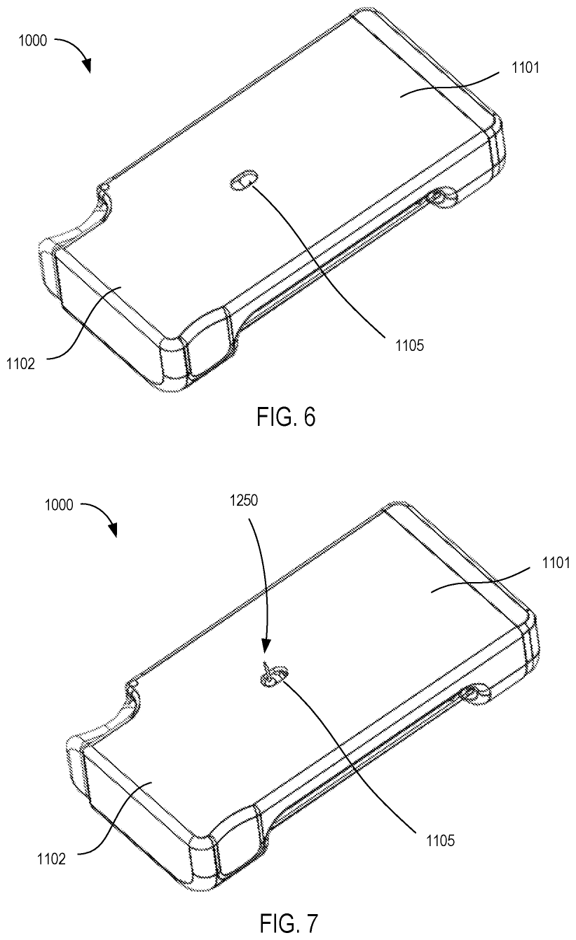

The bottom portion 1104 of the housing 1100 (see FIGS. 6 and 7) includes a contact surface with a needle aperture 1105. The contact surface is configured to contact a body surface of a patient and stabilize the housing 1100 against the body surface during operation. In some embodiments, the contact surface may be provided with an adhesive patch or material to further secure the bottom portion 1104 of the housing 1100 to the body surface of a patient. In some embodiments, a cover or a guard (not shown) may be provided over the needle aperture 1105 to prevent ingress of foreign matter into the housing 1100 through the needle aperture 1105, to maintain needle sterility, and/or to prevent accidental needle prick. In some embodiments, the adhesive patch or material may include a protective film or backing that may be removed by the patient prior to securing the bottom portion 1104 onto the body surface of the patient. In some embodiments, the protective film is coupled to the needle aperture's cover or guard such that removal of the protective film from the adhesive patch also removes the cover or guard from the needle aperture. In some embodiments, a portion of the protective film is attached securely to the activation input member 1510 to prevent actuation of the activation input member 1510 until the protective film is removed from the bottom portion 1104 and/or removed from the activation input member 1510.

FIGS. 10-19 provide an overview of the medicament container assembly 1200, the system actuator assembly 1500, the medicament delivery mechanism 1300, and the flow restriction assembly 1430 (which functions as a delivery control mechanism) of the medicament delivery device 1000. Referring to FIG. 10, the medicament container assembly 1200 has a container body 1210 with a first (i.e., proximal) end portion 1211 and a second (i.e., distal) end portion 1212. The container body 1210 defines a volume that contains (i.e., is filled with or partially filled with) a medicament. The distal end portion 1212 of the medicament container assembly 1200 includes a neck 1213 that is coupled to the needle assembly 1250, as described below. The proximal end portion 1211 of the medicament container assembly 1200 includes an elastomeric member 1217 (i.e., a plunger) that seals the medicament within the container body 1210. The elastomeric member 1217 is configured to move within the container body to convey the medicament from the medicament container assembly 1200. More particularly, as shown in FIG. 14-17, pressure in the medicament container gas chamber exerts a force on a proximal surface 1218 of the elastomeric member 1217 to move the elastomeric member 1217 within the container body 1210 (i.e., to expel the medicament therefrom). Similarly stated, when the elastomeric member 1217 is exposed to a force (e.g., produced by the pressurized gas within the medicament container gas chamber 1440 acting directly on the proximal surface 1218), the elastomeric member 1217 moves distally (i.e., towards the second end portion of the device).

The elastomeric member 1217 can be of any design or formulation suitable for contact with the medicament. For example, the elastomeric member 1217 can be formulated to minimize any reduction in the efficacy of the medicament that may result from contact (either direct or indirect) between the elastomeric member 1217 and the medicament. For example, in some embodiments, the elastomeric member 1217 can be formulated to minimize any leaching or out-gassing of compositions that may have an undesired effect on the medicament. In other embodiments, the elastomeric member 1217 can be formulated to maintain its chemical stability, flexibility and/or sealing properties when in contact (either direct or indirect) with the medicament over a long period of time (e.g., for up to six months, one year, two years, five years or longer).

In some embodiments, the elastomeric member 1217 can be constructed from multiple different materials. For example, in some embodiments, at least a portion of the elastomeric member 1217 can be coated. Such coatings can include, for example, polydimethylsiloxane. In some embodiments, at least a portion of the elastomeric member 1217 can be coated with polydimethylsiloxane in an amount of between approximately 0.02 mg/cm² and approximately 0.80 mg/cm².

The proximal end portion 1211 of the container body 1210 includes a flange 1214 (see e.g., FIG. 10) configured to be disposed within a portion of the medicament cavity 1139. The flange 1214 can be of any suitable size and/or shape. Although shown as substantially circumscribing the container body 1210, in other embodiments, the flange 1214 can only partially circumscribe the container body 1210. In yet other embodiments, the container body 1210 need not include any flange (see, e.g., the container body 2210 described herein).

The medicament container assembly 1200 can have any suitable size (e.g., length and/or diameter) and can contain any suitable volume of the medicament. In some embodiments, the medicament container assembly 1200 (and any of the medicament container assemblies described herein) can be a cartridge having a sealed end portion. The medicament container assembly 1200 can be constructed from any suitable materials including but is not limited to, glass, cyclic olefin copolymer (COC), and cyclic olefin polymers (COP).

In other embodiments, the medicament container assembly 1200 (and any of the medicament container assemblies described herein) can be a prefilled (or prefillable) syringe, such as those manufactured by Becton Dickinson, Gerresheimer, Ompi Pharma or others. For example, in some embodiments, the medicament container assembly 1200 (and any of the medicament container assemblies described herein) can be a Becton Dickinson “BD Hypak Physiolis” prefillable syringe containing any of the medicaments described herein. The medical injector 4000 can be configured to inject any suitable dosage such as, for example, a dose of up to 4 mL of any of the medicaments described herein. In other embodiments, the medicament delivery device 1000 can be configured to inject a dose of up to 2 mL, 3 mL, 4 mL, 5 mL, 6 mL, 7 mL, 8 mL, 9 mL, 10 mL, or more of any of the medicaments described herein.

The container body 1210 can be constructed from glass, and can be fitted and/or coupled to any suitable needle. For example, in some embodiments, the container body 1210 can be coupled to a needle (e.g., via the needle coupling member 1251 or a direct coupling) having any suitable size. Any of the medicament container assemblies and/or prefilled syringes described herein can be coupled to a needle (via the needle assembly) having a gauge size of 21 gauge, 22 gauge, 23 gauge, 24 gauge. 25 gauge, 26 gauge, 27 gauge, 28 gauge, 29 gauge, 30 gauge, or 31 gauge. Any of the medicament container assemblies and/or prefilled syringes described herein can be coupled to a needle having any suitable length, such as, for example, a length of about 0.2 inches, about 0.27 inches, about 0.38 inches, about 0.5 inches, about 0.63 inches, about 0.75 inches, or more. In some embodiments, for example, any of the medicament containers and/or prefilled syringes described herein can be coupled to a 29 gauge, needle having a length of approximately 0.5 inches.

As shown in FIGS. 9-11 and 14-17, the neck 1213 of the container body 1210 is inserted into a receiving portion 1254 at a first end portion 1252 of the needle assembly 1250. The receiving portion 1254 includes an inner circumferential surface and an O-ring for sealing and securing the neck 1213 of the container body 1210.

As shown in FIGS. 10 and 11, the system actuator assembly 1500 includes an actuator input member 1510, a release member 1550, and an actuator spring 1576. The release member 1550 has a first (or proximal) end portion 1551 and a second (or distal) end portion 1552, and is movably disposed within the distal end portion of the gas container cavity 1151. The proximal end portion of the release member 1550 includes a sealing member 1574 and a puncturer 1575. The sealing member 1574 is configured to engage the sidewall of the housing 1100 defining the gas container cavity 1151 such that the proximal end portion of the gas container cavity 1151 is fluidically isolated from the distal end portion of the gas container cavity 1151. In this manner, when gas is released from the gas container 1410, the gas contained in the proximal end portion of the gas container cavity 1151 is unable to enter the distal end portion of the gas container cavity 1151. The puncturer 1575 of the release member 1550 is configured to contact and puncture a frangible seal 1413 on the gas container 1410 when the release member 1550 moves proximally within the gas container cavity 1151.

As shown in FIG. 10A, the distal end portion 1552 of the release member 1550 includes one or more extensions 1553. The extensions 1553 have projections that include tapered surfaces and engagement surfaces. Further, the extensions 1553 define an opening between the adjacent extensions 1553. The engagement surfaces of the extensions 1553 are configured to contact the release member contact surface 1146 of the housing 1100 and to pass through a release member aperture 1145 when the extensions 1553 are compressed inwards. In this manner, the engagement surfaces limit proximal movement of the release member 1550 in the normal, uncompressed state. The opening 1554 defined by the extensions 1553 is configured to allow the extensions 1553 to flex and retract the engagement surfaces of the extensions 1553 inwards. An opening 1554 is defined between the extensions 1553. In some embodiments, a safety pin (not shown) can be inserted into the opening 1554 to prevent the extensions 1553 from moving, thereby disabling the release member 1550 until the safety pin is removed.

The tapered surfaces of the extensions 1553 are configured to contact corresponding tapered or conical surfaces 1557 of the actuator input member 1510 when the actuator input member 1510 is moved from a first position (i.e., released position or home position) to a second position (i.e., depressed position or active position). For example, as shown in FIGS. 10, 10A and 11, as the actuator input member 1510 is depressed by an operator, the actuator input member 1510 rotates clockwise about support pin 1511. As the actuator input member 1510 is rotated, the tapered or conical surfaces 1557 contact the extensions 1553. Continued movement of the actuator input member 1510 causes the extensions 1553 to flex inward until the engagement surfaces of the extensions 1553 are no longer in contact with the release member contact surface 1146 of the housing 1100. Force applied by the actuator spring 1576 on the proximal end portion 1551 against the housing 1100 causes the extensions 1553 to pass through the release member aperture 1145 and the release member 1550 to move proximally within the gas container cavity 1151 towards the gas container 1410.

As shown in FIG. 2, the housing defines a contoured grip surface opposite the actuator input member 1510. In this manner, the user can grasp the grip surface (e.g., with the thumb) and use their opposing finger(s) to depress the actuator input member 1510. Alternatively, the user can depress the actuator input member 1510 with the thumb and use their opposing finger(s) to grasp the grip surface. This arrangement allows the actuation force applied by the user to be applied in a direction nonparallel to the needle 1260. Specifically, the actuation force is applied substantially parallel to the target surface (and the contact surface of the housing 1100). Thus, the actuation force applied by the user does not press downward (or into) the target surface, which can reduce discomfort. Additionally, by limiting the depression of the target tissue, the needle delivery depth and characteristics can be more consistent. Additionally, by orienting the actuator input member 1510 to receive a force that is perpendicular or generally perpendicular to the direction (e.g., downward) in which that the medicament delivery device 1000 is placed onto the target surface, any downward force applied on the medicament delivery device 1000 during positioning onto the target surface will not inadvertently actuate the input member 1510 and prematurely start the medicament delivery device 1000.

The gas container 1410 includes a second (or distal) end portion 1411 and a first (or proximal) end portion 1412, and is configured to contain and/or produce a pressurized gas. The distal end portion 1411 of the gas container 1410 contains a frangible seal 1413 configured to break when the puncturer 1575 of the release member 1550 contacts the frangible seal 1413. The gas container retention member 1180 of the housing cap 1110 of the housing 1100 is configured to receive and/or retain the proximal end portion 1412 of the gas container 1410. Said another way, the position of the gas container 1410 within the gas container cavity 1151 is maintained by the gas container retention member 1180.

As shown in FIGS. 10 and 11, the length of the gas container retention member 1180 and the length of the release member 1550 collectively determine the distance between the puncturer 1575 and the frangible seal 1413 when the medicament delivery device 1000 is in the storage configuration. Accordingly, this distance, which is the distance through which the puncturer 1575 travels when the medicament delivery device 1000 is actuated via the actuator input member 1510, can be adjusted by changing the length of the gas container retention member 1180 and/or the length of the release member 1550. In some embodiments, the actuation time and/or the force exerted by the puncturer 1575 on the frangible seal 1413 can be adjusted by changing the distance between the puncturer 1575 and the frangible seal 1413.

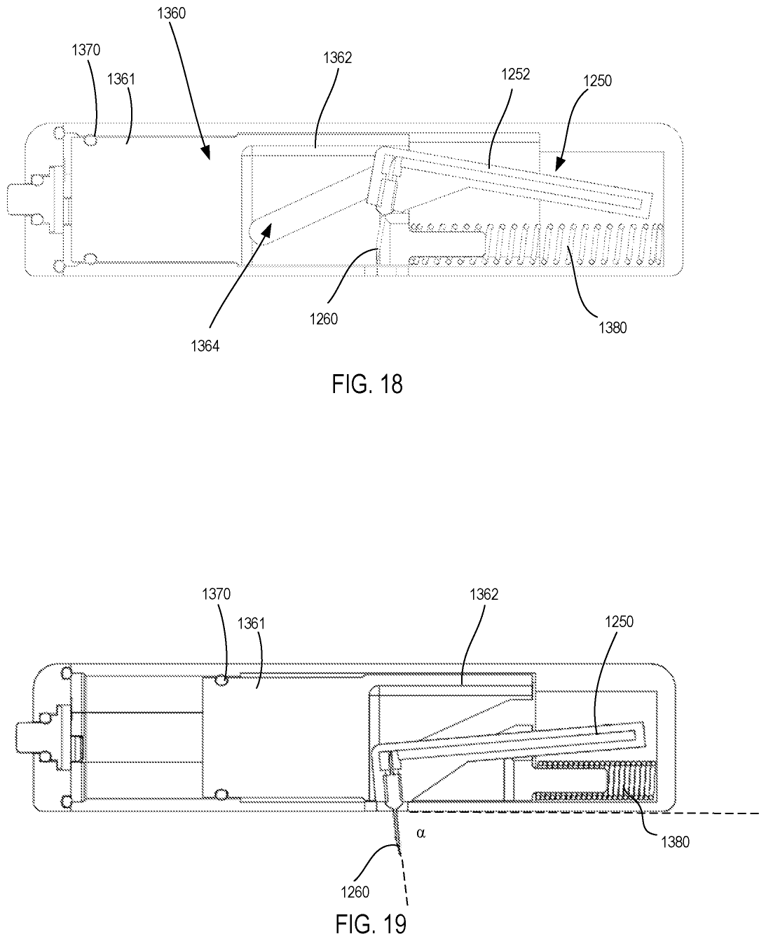

As shown in FIGS. 12-14, the medicament delivery mechanism 1300 includes an insertion member 1360, a flow restriction assembly 1430 (also referred to as a delivery control mechanism), a gas vent assembly 1310, and a needle assembly 1250. The insertion member 1360, gas vent assembly 1310, and needle assembly 1250 are each movably disposed within the housing 1100. The insertion member 1360 is movable within the delivery mechanism cavity 1161 (see e.g., FIGS. 14-17). The gas vent assembly 1310 is movable within the gas passageway 1135 (see e.g., FIGS. 16 and 17). As shown in FIGS. 18 and 19, at least a portion of the needle assembly 1250 is rotatable within the delivery mechanism cavity 1161 to move a needle 1260 of the needle assembly 1250 from a first needle orientation (i.e., retracted orientation) to a second needle orientation (i.e., deployed orientation).

The insertion member 1360 includes a first (or proximal) end portion 1361, a second (or distal) end portion 1362, and defines a groove 1363 (see e.g., FIGS. 18-20). The groove is configured to support an O-ring 1370. The O-ring is configured to engage the sidewall of the housing 1100 defining the delivery mechanism cavity 1161 such that the proximal end portion of the delivery mechanism cavity 1161 is fluidically isolated from the distal end portion of the delivery mechanism cavity 1161. In this manner, when the gas is released from the gas container 1410, the gas conveyed to the proximal end portion of the delivery mechanism cavity 1161 is unable to enter the distal end portion of the delivery mechanism cavity 1161. As described below, the proximal end portion 1361 of the insertion member 1360 includes a proximal surface 1376 which forms a portion of the boundary of the housing gas chamber (i.e., the portion of delivery mechanism cavity 1161).

As shown in FIGS. 12, 13 and 20, the distal end portion 1362 includes a contact portion 1364 for engaging and moving at least a portion of the needle assembly 1250. The contact portion 1364 defines a first guide channel 1365 and a second guide channel 1366. Although the first guide channel 1365 and the second guide channel 1366 are depicted as extending along a linear path, in some embodiments, the first and second guide channels 1365, 1366 can define a curvilinear path to alter the deployment and retraction characteristics of the needle 1260. In some embodiments, the contact portion 1364 extends at an angle (i.e., diagonally) as it traverses from the distal end portion 1362 towards the proximal end portion 1361. Stated differently, the contact portion 1364 extends along an axis that is non-parallel with and non-perpendicular to a central axis of the insertion member 1360. The distal end portion 1362 also includes a protrusion 1367 about which the retraction spring 1380 is disposed.

The needle assembly 1250 includes a needle coupling member 1251 with a first end portion 1252 and a second end portion 1253 (see e.g., FIGS. 21-23). The needle assembly 1250 includes the receiving portion 1254 for receiving a portion of the medicament container, such as the neck 1213 of the medicament container assembly 1200 (see e.g., FIG. 10). As shown, the receiving portion 1254 includes a seal 1258 to maintain a fluid coupling between the medicament container assembly 1200 and the needle assembly 1250 without leakage. In other embodiments, the receiving portion need not include a seal. In some embodiments, the receiving portion includes a puncturer (not shown) to puncture a frangible seal at the distal end of the medicament container assembly 1200 upon insertion into the receiving portion 1254. In other embodiments, the receiving portion includes a septum that is pierced by a needle coupled to the medicament container (e.g., in a pre-filled syringe configuration) to place the medicament container assembly 1200 in fluid communication with the needle assembly 1250. In some embodiments, the receiving portion 1254 is configured to receive a luer lock fitting.

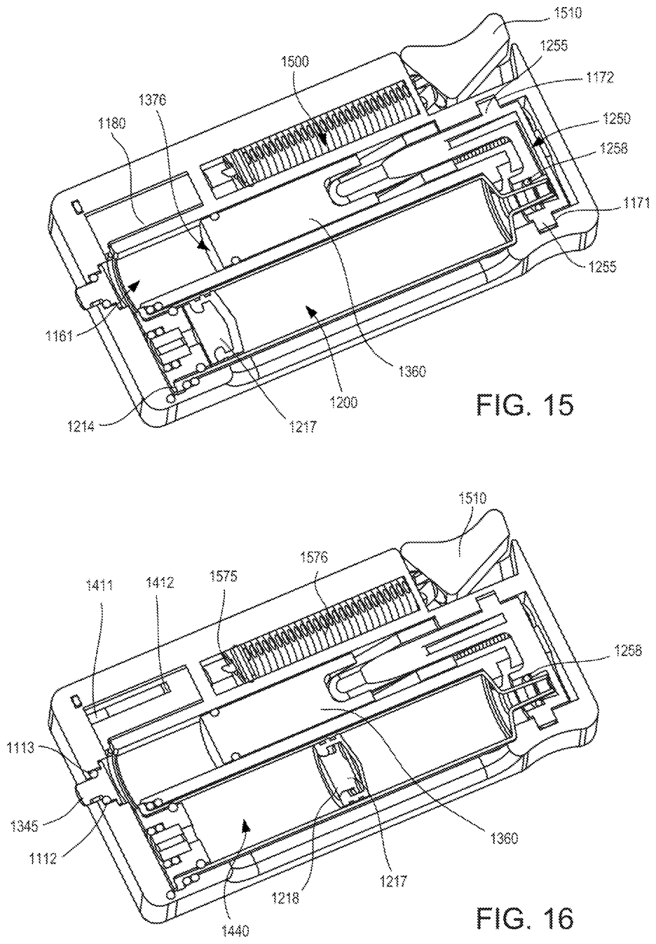

The receiving portion 1254 includes a central axis. The first end portion 1252 includes a pair of mounting bosses 1255 extending perpendicularly relative to the central axis of the receiving portion 1254 (see e.g., FIG. 23). As shown in FIGS. 14 and 15, the pair of mounting bosses 1255 are rotatably supported by annular recesses 1171, 1172 of the housing. Stated differently, the pair of mounting bosses 1255 can be cylindrical protrusions extending from the first end portion 1252. The annular recess 1171 can be a blind hole formed in medicament cavity 1139 of the housing 1100 and the annular recess 1172 can be a blind hole formed in the delivery mechanism cavity 1161. The pair of mounting bosses 1255 define a rotational axis (A_{R}) about which the needle assembly 1250 rotates during operation.

The second end portion 1253 of the needle coupling member 1251 includes a needle support portion 1256 for supporting the needle 1260. The needle support portion 1256 includes a central axis. The second end portion 1253 includes a pair of guide bosses 1257 extending perpendicularly relative to the central axis of the needle support portion 1256. In some embodiments, the pair of mounting bosses 1255 are parallel with the pair of guide bosses 1257. The pair of guide bosses 1257 are configured to engage and ride along the first guide channel 1365 and the second guide channel 1366 (see e.g., FIGS. 12 and 13). Stated in a different manner, the ramped nature of the first and second guide channels 1365, 1366 causes the pair of guide bosses 1257 to move orthogonally relative to a longitudinal axis of the delivery mechanism cavity 1161.

When the device 1000 actuated, pressurized gas flows into the housing gas chamber and within the delivery mechanism cavity 1161 in a first phase of expansion. In this manner, the pressurized gas produces a force on the proximal surface 1376 of the insertion member 1360, which moves the insertion member 1360 distally within the housing 1100. When the pressurized gas produces a force on the proximal surface 1376, the force from the pressurized gas is high enough such that the insertion member 1360 overcomes a force applied by the retraction spring 1380. As a result, the insertion member 1360 moves distally within the delivery mechanism cavity 1161 of the housing 1100. As the insertion member 1360 moves distally, the first and second guide channels 1365, 1366 advance distally within the housing 1100 causing the guide bosses 1257 to be moved downward towards the bottom portion 1104 of the housing 1100. In turn, the needle assembly 1250 is rotated about the rotational axis (A_{R}). In some embodiments, the needle assembly 1250 is rotated about the rotational axis (A_{R}) by about 5 degrees to about 45 degrees while the needle 1260 is moved from the retracted orientation to the deployed orientation. In some embodiments, the needle assembly 1250 is rotated about the rotational axis (A_{R}) by about 10 degrees to about 30 degrees. In some embodiments, the needle 1260 extends at a non-orthogonal angle relative to a plane of the bottom portion 1104 of the housing 1100. In some embodiments, the needle 1260 extends at an angle (a) of between about 80 degree to about 88 degree relative to the plane of the bottom portion 1104 (see e.g., FIG. 19). In some embodiments, the needle 1260 extends at an angle (a) of between about 75 degree to about 87 degree relative to the plane of the bottom portion 1104.

Once the needle 1260 has been placed in the deployed orientation, gas pressure continues to build up within the housing gas chamber (e.g., in the proximal portions of the delivery mechanism cavity 1161 and medicament cavity 1139). Gas flow (and pressure) to deliver the medicament is regulated by the flow restriction assembly 1430. As shown in FIGS. 14A, 15 and 16, the flow restriction assembly 1430 includes a first body portion 1431 and a second body portion 1432. The first body portion 1431 includes a flow restriction retainer 1433 configured to support at least a portion of a flow restriction member 1450. The flow restriction retainer 1433 includes a cylindrical inner surface 1434 and an end surface with a through-hole 1435 extending into an interior portion of the second body portion 1432.

In this manner, the interior of the second body portion 1432 is in fluid communication with the first body portion 1431. Although the through-hole 1435 is shown as being coaxial with a center of the flow restriction member 1450, in some embodiments, the through-hole 1435 can be non-coaxial with the flow restriction member 1450. In some embodiments, at least a portion of a flow restriction element 1452 overlaps with a portion of through-hole 1435. In some embodiments, at least 50% of the flow restriction element 1452 overlaps with the through-hole 1435.

As shown in FIG. 14A, the flow restriction member 1450 includes a sleeve member 1451 and a flow restriction element 1452, and the flow restriction element 1452 is supported within the sleeve member 1451. In some embodiments, the sleeve member 1451 is a metal sleeve. In some embodiments, the metal sleeve is made of stainless steel or brass. In some embodiments, the flow restriction element 1452 is a porous material. In some embodiments, the porous material is sintered porous metal. In some embodiments, the flow restriction element 1452 is calibrated with nitrogen gas (N₂) at 30 psig (inlet side) to atmosphere (outlet side) at standard temperature and pressure to have a flow rate of between 0.01 to 3 standard cubic centimeter per minute (sccm). In some embodiments, the flow restriction member 1450 is calibrated with nitrogen gas (N₂) at 30 psig (inlet side) to atmosphere (outlet side) at standard temperature and pressure to have a flow rate of between about 0.75 and 1.5 standard cubic centimeter per minute (sccm). In some embodiments, the flow restriction member 1450 is calibrated with nitrogen gas (N₂) at 30 psig (inlet side) to atmosphere (outlet side) at standard temperature and pressure to have a flow rate of about 1 standard cubic centimeter per minute (sccm). As described herein, standard temperature is 60° F. (15.6° C.) and standard pressure is 14.696 psia (101.3 kPa).

In some embodiments, the compressed gas supplied by the gas container 1410 is an argon gas and the flow restriction element 1452 has a flow rate rating of about 0.75 and 1.5 sccm based on the nitrogen gas calibration described above. In some embodiments, the compressed gas supplied by the gas container 1410 is an argon gas and the flow restriction element 1452 has a flow rate rating of about 1 sccm based on the nitrogen gas calibration described above. In some embodiments, the compressed gas in the gas container 1410 has a molecular weight greater than the molecular weight of argon. For example, in some embodiments, the compressed gas supplied by the gas container 1410 is R134a (Tetrafluoroethane) and the flow restriction element 1452 has a flow rate rating of about 10 to 100 sccm based on the nitrogen gas calibration described above. In some embodiments, the compressed gas supplied by the gas container 1410 is R134a (Tetrafluoroethane) and the flow restriction element 1452 has a flow rate rating of about 20 to 40 sccm based on the nitrogen gas calibration described above.

In some embodiments, the flow rate of the medicament can be reduced to less than 0.2 mL/sec (or in some embodiments between 0.05 mL/sec and 0.01 mL/see) using gas pressure that is initially supplied to medicament cavity 1139 and through the flow restriction member 1450. The lower injection forces and/or slower delivery (compared with pressures supplied directly from the medicament cavity 1139 to the elastomeric member) can produce laminar flow of the medicament through the needle, prevent shearing of high molecular weight compounds in the medicament, and/or reduce pain sensed by a patient particularly if the medicament being delivered is very high viscosity (e.g., greater than about 100 centipoise at room temperature). In some embodiments, a screen or mesh protective member can be provided on a proximal side of the flow restriction member 1450 to prevent any particulate or debris from clogging the flow restriction element 1452 during operation.

As shown in FIG. 14A, the second body portion 1432 includes an outer cylindrical surface. The outer cylindrical surface includes a groove for supporting an O-ring 1436. The second body portion 1432 is configured to be inserted into a proximal end of the medicament container assembly 1200. The O-ring 1436 prevents pressurized gas in the proximal portion of the medicament cavity 1139 from passing between the outer cylindrical surface of the second body portion 1432 and an internal wall of the medicament container assembly 1200. Stated differently, the O-ring 1436 seals the medicament container assembly 1200 from the housing gas chamber portion of the medicament cavity 1139.

The inner diameter of the cylindrical inner surface 1434 is greater than an inner diameter of the second body portion 1432. The first body portion 1431 further includes a flange portion 1431F extending radially from an outer surface of the first body portion 1431. The flange portion 1431F is configured to mount onto the flange 1214 of the medicament container body 1210. One or more O-rings 1437, 1438 are supported on the cylindrical inner surface 1434 to prevent pressurized gas from passing between the flow restriction member 1450 and the cylindrical inner surface 1434. In other words, the O-rings 1437, 1438 prevents pressurized gas from bypassing around the flow restriction member 1450. As shown in FIG. 14A, the flange portion 1431F further defines a portion of the gas passageway 1135 through which the pressurized gas can flow into the flow restriction member 1450.

As described above, during the first phase of expansion, gas pressure is applied on the proximal surface 1376 of the insertion member 1360 to move the insertion member 1360 distally within the housing 1100. The distal movement of the insertion member 1360 causes the guide bosses 1257 to move along the first and second guide channels 1365, 1366 until the needle 1260 has been extended by a desired distance from the bottom portion 1104 of the housing 1100. In some embodiments, the flow restriction assembly 1430 (e.g., a flow delivery control mechanism) can permit gas to pass through the flow restriction member 1450 but not build enough pressure to move the elastomeric member 1217 during the first phase of expansion. In some embodiments, the pressurized gas in the medicament cavity 1139 drops to about 90-100 psi after the needle 1260 has been deployed.

As shown in FIGS. 14-17, the gas from the medicament cavity 1139 passes through and is regulated by the flow restriction member 1450 to begin a second phase of expansion. The gas passing through the flow restriction member 1450 travels through the through-hole 1435. After passing through the through-hole 1435, the gas enters a medicament container gas chamber 1440 sealed between a distal side of the O-ring 1436 and a proximal side of the elastomeric member 1217. This causes the elastomeric member 1217 to move in the distal direction within the medicament container body 1210. Distal movement of the elastomeric member 1217 generates a pressure upon the medicament contained within the medicament container assembly 1200, thereby allowing at least a portion of the medicament to flow out of the medicament container assembly 1200 via the needle 1260. The medicament is delivered to a body of a user via the medicament delivery path defined by the medicament container assembly 1200, the internal passage of the needle assembly 1250, and the needle 1260.

As generally shown in FIGS. 14A, 16 and 17, once the elastomeric member 1217 has completed its travel stroke to deliver a desired dose of medicament, the second phase of expansion is complete. To retract the needle 1260, a valve portion 1345 of the gas vent assembly 1310 can be actuated to release internal gas pressure from the delivery mechanism cavity 1161. The valve portion 1345 can be manually depressed into the housing cap 1110 by a user. An internal passage 1346 of the valve portion 1345 passes over the O-ring 1113 thereby placing the gas passageway 1135 and the delivery mechanism cavity 1161 in fluid communication with an external environment. Pressurized gas from within the medicament cavity 1139 and the delivery mechanism cavity 1161 exits through the vent opening 1112 via the internal passage 1346 of the valve portion 1345 (see e.g., FIGS. 14A and 17).

As pressurized gas is released, the force of the retraction spring 1380 becomes greater than the gas pressure applied on the proximal surface 1376 of the insertion member 1360. As a result, the insertion member 1360 moves proximally within the delivery mechanism cavity 1161. As the insertion member 1360 moves proximally, the first and second guide channels 1365, 1366 also move proximally causing the pair of guide bosses 1257 to move upwards and away from the bottom portion 1104 of the housing 1100. In turn, the needle assembly 1250 is rotated about the rotational axis (A_{R}) and the needle 1260 is retracted back within the housing 1100.

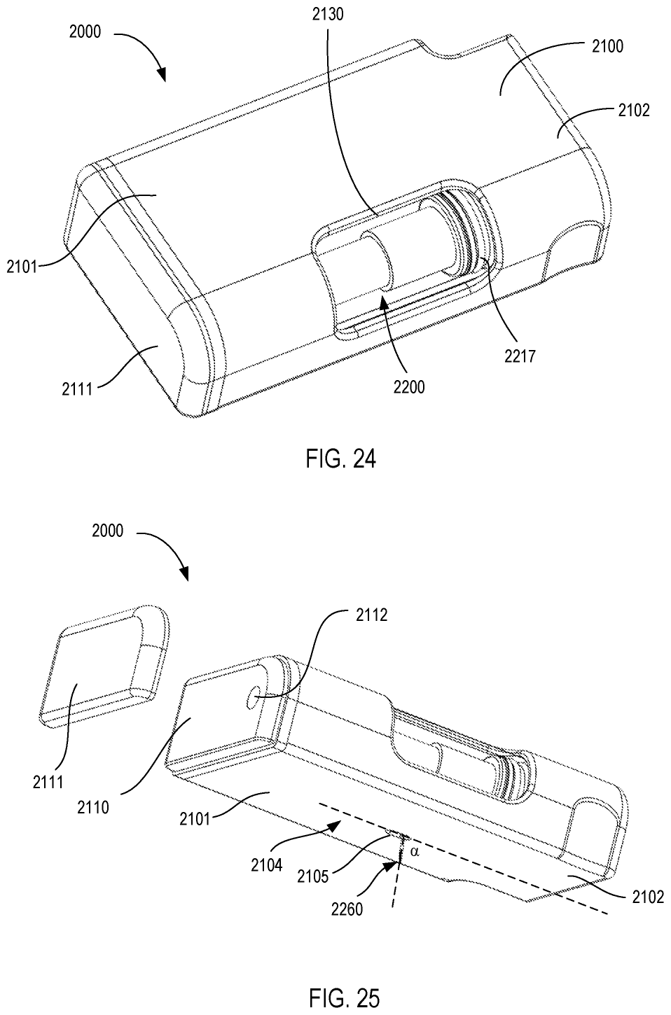

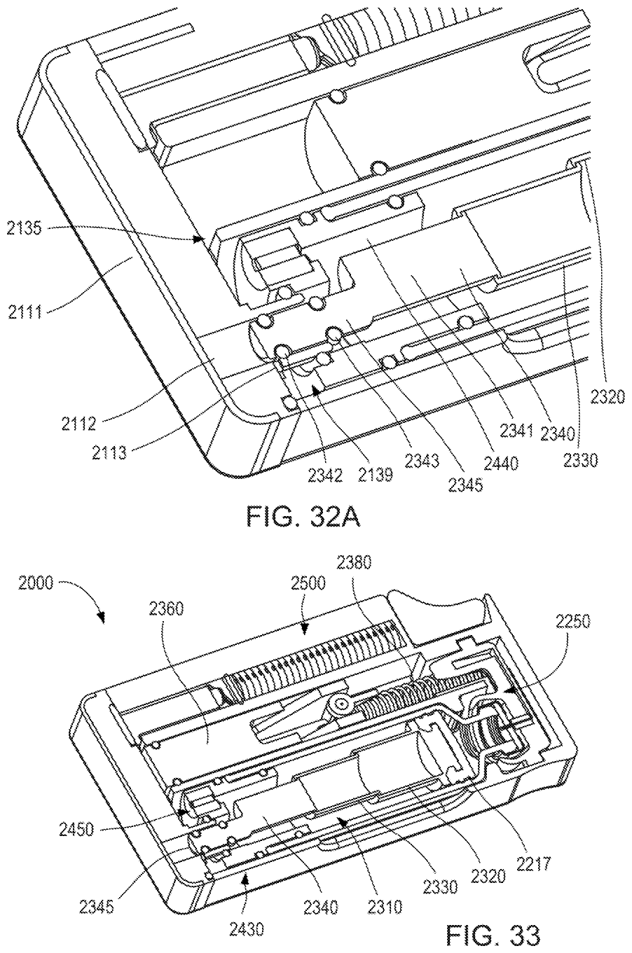

In some embodiments, a medicament delivery device can include a gas release valve that is automatically actuated upon completion of the injection. As shown in FIGS. 24-33, a medicament delivery device 2000 (also referred to as a medical injector) includes a housing 2100, a needle assembly 2250, a medicament delivery mechanism 2300, and system actuation assembly 2500, which are all similar to the corresponding parts in the medicament delivery device 1000 described above. The medical injector further includes a medicament container assembly 2200 configured to actuate within the housing 2100, and a gas vent assembly 2310 configured to retract the needle assembly 2250 at the end of delivery, as will be described in greater detail below.

With reference to FIGS. 24 and 25, the housing 2100 has a first (or proximal) end portion 2101 and a second (or distal) end portion 2102. The housing 2100 includes a top portion 2103 extending between the proximal end portion 2101 and a distal end portion 2102. The top portion 2103 defines a status indicator aperture 2130. The status indicator aperture 2130 can allow a patient to monitor the status and/or contents of the medicament container assembly 2200, a position of an elastomeric member 2217 within the medicament container assembly 2200, and/or and the medicament contained within the housing 2100. For example, by visually inspecting the status indicator aperture 2130, a patient can determine whether the medicament container assembly 2200 contains a medicament and/or whether the medicament has been dispensed. In some embodiments, the status indicator aperture 2130 includes a length to display an entire, or substantially the entire, stroke length of the elastomeric member 2217 during operation. Moreover, as shown in FIG. 24, a portion of the gas vent assembly 2310 that expands within the medicament container assembly 1200 is visible through the status indicator aperture 2130 to provide further visual indicator that injection has been completed.

The housing 2100 includes a bottom portion 2104 extending between the proximal end portion 2101 and a distal end portion 2102. The bottom portion 2104 includes a contact surface for contacting a body surface of a patient. The bottom portion 2104 includes a needle aperture 2105 configured to allow a needle 2260 of the needle assembly 2250 to pass through during operation (see e.g., FIG. 25). In some embodiments, the bottom portion 2104 includes an adhesive material for temporarily securing the bottom portion 2104 to the body surface of the patient. In some embodiments, the contact surface may be provided with an adhesive patch or material to further secure the bottom portion 2104 of the housing 2100 to the body surface of a patient. In some embodiments, a cover or a guard (not shown) may be provided over the needle aperture 2105 to prevent ingress of foreign matter into the housing 2100 through the needle aperture 2105, to maintain needle sterility, and/or to prevent accidental needle prick. In some embodiments, the adhesive patch or material may include a protective film or backing that may be removed by the patient prior to securing the bottom portion 2104 onto the body surface of the patient. In some embodiments, the protective film is coupled to the needle aperture cover or guard such that removal of the protective film from the adhesive patch also removes the cover or guard from the needle aperture. In some embodiments, a portion of the protective film is attached securely to the activation input member 2510 to prevent actuation of the activation input member 2510 until the protective film is removed from the bottom portion 2104.

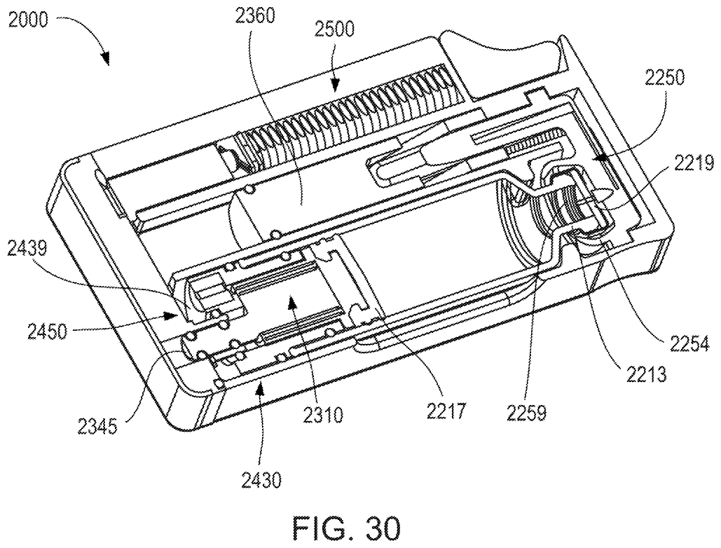

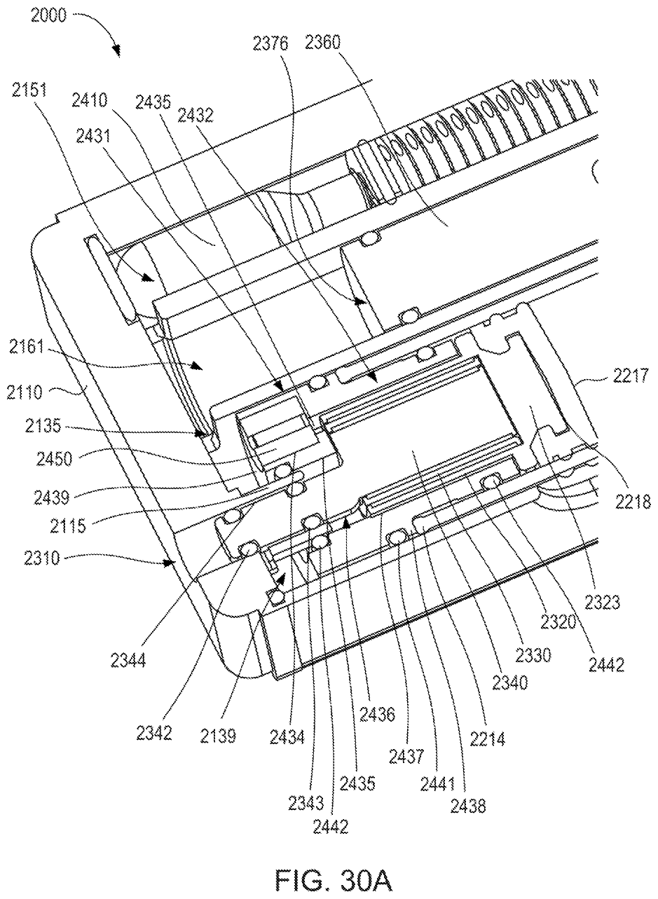

The housing 2100 defines a medicament cavity 2139, a gas container cavity 2151, and a delivery mechanism cavity 2161 (see e.g., FIG. 30A). The gas container cavity 2151 is configured to receive the gas container 2410 (which can be the same as gas container 1410 or any other gas container described herein) and a portion of the system actuator assembly 2500 (see e.g., FIG. 27). The proximal end portion of the gas container cavity 2151 is configured to support the gas container retention member 2180 (which can be the same as gas container retention member 1180, or any other gas container retention member described herein). The gas container cavity 2151 is in fluid communication with the medicament cavity 2139 and the delivery mechanism cavity 2161 via a gas passageway 2135 defined in the housing 2100, as described in further detail herein (see e.g., FIG. 30A).

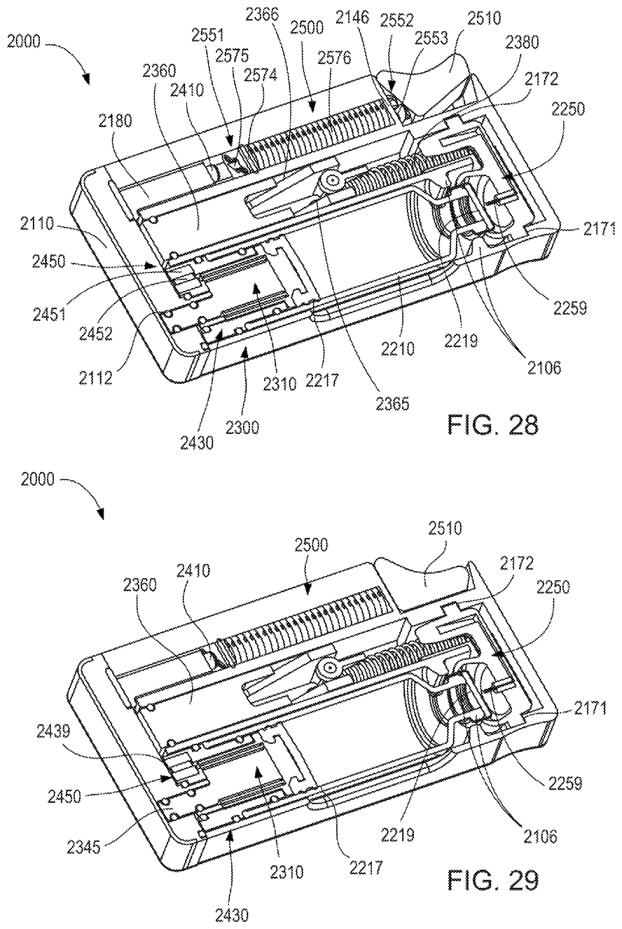

The medicament cavity 2139 is configured to receive the medicament container assembly 2200 and at least a portion of the medicament delivery mechanism 2300. The medicament delivery mechanism 2300 can be the same as the medicament delivery mechanism 1300 of the medical injector described above. The medicament delivery mechanism includes an insertion member 2360 (also referred to as a needle assembly carrier) and a needle assembly 2250 (see e.g., FIGS. 25 and 27). The medicament cavity 2139 is in fluid communication with the gas container cavity 2151, the delivery mechanism cavity 2161, and a vent opening 2112 (see e.g., FIGS. 27 and 28).

The first (or proximal) end portion 2101 of the housing 2100 includes a housing cap 2110 and a cap cover 2111 (see e.g., FIG. 25). A gas container retention member 2180 is configured to be inserted into the gas container cavity 2151 to retain a gas container 2410 that contains a pressurized gas (see e.g., FIG. 27). The housing cap 2110 is configured to retain the gas container retention member 2180 within the gas container cavity 2151. When the medical injector 2000 is actuated, pressurized gas from the gas container 2410 is conveyed from the gas container cavity 2151 to the medicament cavity 2139 and to the delivery mechanism cavity 2161 via the gas passageway 2135 of the housing 2100 (see e.g., FIG. 30A). Said another way, the gas passageway 2135 places the gas container cavity 2151 in fluid communication with the medicament cavity 2139 and the delivery mechanism cavity 2161. Thus, the pressurized gas from the gas container 2410 is conveyed from the gas container cavity 2151 to the delivery mechanism cavity 2161 to move the insertion member 2360 and the needle assembly 2250. As will be described in greater detail below, the pressurized gas from the gas container 2410 is conveyed from the gas container cavity 2151 to a proximal portion of the medicament cavity 2139 to actuate the medicament container assembly 2200. The proximal portion of the medicament cavity 2139 also serves as a pressurized gas reservoir used to inject the medicament, as described herein.

The housing cap 2110 includes a vent opening 2112 that can be selectively placed in fluid communication with the gas passageway 2135 within the housing 2100. The housing cap 2110 also includes a cap cover 2111 coupled to a proximal end portion of the housing cap 2110 while retaining a gap between the proximal end portion of the housing cap 2110 and the cap cover 2111. The cap cover 2111 prevents the vent opening 2112 from direct external contact and prevents clogging from external debris. The vent opening 2112 provides the passageway through which pressurized gas is conveyed from gas passageway 2135 (including from within the medicament cavity 2139 and the delivery mechanism cavity 2161) to a volume outside of the medical injector 2000. The vent opening 2112 and the gap between the housing cap 2110 and the cap cover 2111 allows pressurized gas from within the medicament cavity 2139 to escape out to the volume outside the medical injector 2000. In this manner, the force produced by the pressurized gas on the medicament delivery mechanism 2300 and/or the medicament container assembly 2200 (via the delivery control assembly 2430) can be reduced to allow needle retraction after the injection is completed.

Although the vent opening 2112 is shown as being defined by the housing cap 2110, and being in a proximal surface thereof, in other embodiments, the vent opening 2112 (and any of the vent openings described herein) can be defined within any suitable portion of the housing cap or side wall. For example, in some embodiments, the vent opening 2112 (and any of the vent openings described herein) can be defined by the housing cap 2110, but can have a centerline that is nonparallel to a longitudinal axis of the medical injector 2000. Said another way, in some embodiments, the vent opening 2112 (and any of the vent openings described herein) can open towards a side of the medical injector, rather than opening towards the proximal end, as shown. In other embodiments, the vent opening 2112 (and any of the vent openings described herein) can be defined by any wall and/or surface of the housing 2100.

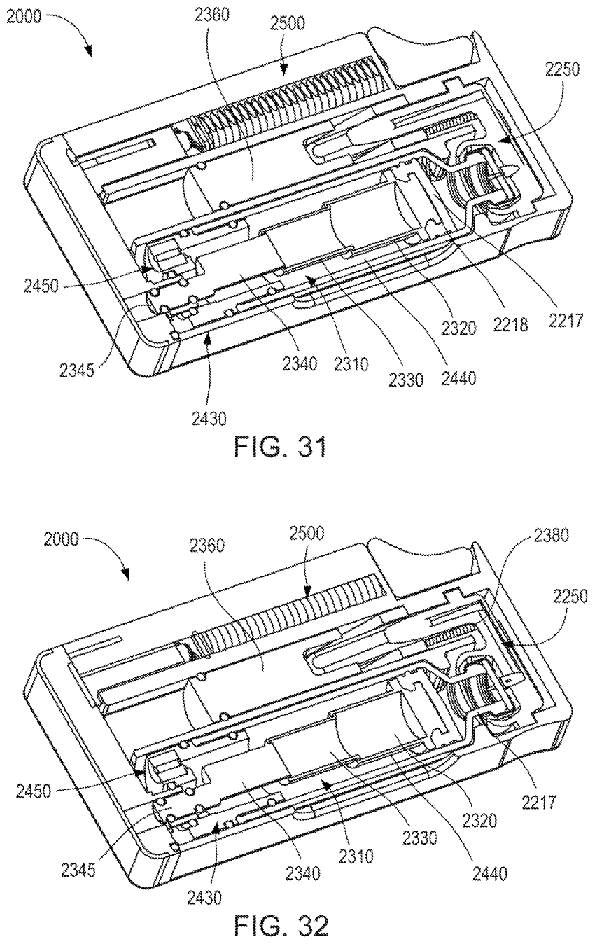

The housing cap 2110 includes a guide wall 2115 within which a guide member of the gas vent assembly 2310 moves to selectively place the vent opening 2112 in fluid communication with the gas passageway 2135 within the housing (see e.g., FIGS. 31 and 32). Specifically, the guide wall 2115 defines an inner cylindrical wall surface within which a valve portion 2345 of the third member 2340 (see e.g., FIGS. 30A and 32A) slides during operation. The guide wall 2115 defines an outer cylindrical wall about which a guide surface of the delivery control assembly 2430 slides during actuation of the medicament container assembly 2200. As shown in FIG. 28, the distal portion of the medicament cavity 2139 includes a shoulder portion 2106 with a contact surface. The contact surface of the shoulder portion 2106 is configured to limit distal movement of the medicament container body 2210 as will be described in further detail below.

FIGS. 27-33 provide an overview of the medicament container assembly 2200, the system actuator assembly 2500, the medicament delivery mechanism 2300, and the delivery control assembly 2430 (which functions to couple the medicament container assembly 2200 to the medicament delivery mechanism 2300, and function as a delivery control mechanism and a medicament container actuator) of the medical injector 2000.

Referring to FIGS. 27 and 28, the medicament container assembly 2200 has a container body 2210 with a first (or proximal) end portion 2211 and a second (or distal) end portion 2212. The container body 2210 defines a volume that contains (i.e., is filled with or partially filled with) a medicament. The distal end portion 2212 of the medicament container assembly 2200 includes a neck 2213 that is coupled to the needle assembly 2250, as described below. The proximal end portion 2211 of the container assembly 2200 includes an end surface 2214. The end surface 2214 can be of any suitable size and/or shape, and in some embodiments, can be a flange similar to the flange 1214 described above. The proximal end portion 2211 of the medicament container assembly 2200 further includes an elastomeric member 2217 (i.e., a plunger) that seals the medicament within the container body 2210. The elastomeric member 2217 is configured to move within the container body to convey the medicament from the medicament container assembly 2200. The neck 2213 includes an external surface configured to abut against the contact surface of the shoulder portion 2106 to limit distal travel of the container body 2210 within the medicament cavity 2139 (and also within the receiving portion 2254).

Referring to FIG. 30A, the delivery control assembly 2430 (also referred to as a delivery control mechanism) includes a first body portion 2431 and a second body portion 2432. The first body portion 2431 includes a flow restriction retainer 2433 and a vent mechanism passageway 2436. The flow restriction retainer 2433 includes a cylindrical inner surface 2434 and an end surface with a through-hole 2435 extending into an interior portion of the second body portion. The flow restriction retainer 2433 is configured to support at least a portion of a flow restriction member 2450. The second body portion 2432 includes a housing configured to house at least a portion of the gas vent assembly 2310. The housing includes a cylindrical inner surface 2437. Although the cylindrical inner surface 2434, gas vent passageway 2436, the cylindrical inner surface 2437 are shown as having a circular cross-section, one or more of the cylindrical inner surface 2434, gas vent passageway 2436, the cylindrical inner surface 2437 can be configured to have an oval, elliptical, or other rounded cross-sections. The gas vent assembly 2310 includes a first (or distal member) member 2320, a second (or intermediate) member 2330, and a third (or proximal) member 2340. These components are nested together such that the gas vent assembly 2310 can be transitioned from a collapsed configuration (see e.g., FIGS. 30 and 30A) to an expanded configuration (see e.g., FIGS. 31-33) as will be described in greater detail below. In some embodiments, the elastomeric member 2217 and one or more portions of the gas vent assembly 2310 can be color coded to provide a user with visual feedback on the progress of the medicament delivery process. For example, one or more of the distal member 2320, the intermediate member 2330, and/or the third member 2340 can be color coded red or orange such that it shows through the status indicator aperture 2130 to indicate that delivery is in process. In some embodiments, the elastomeric member 2217 can include a green color to indicate that medicament delivery is in process as the elastomeric member 2217 progresses within the medicament container body 2210. The status indicator aperture 2130 can be sized to obscure or hid the elastomeric member 2217 when the elastomeric member 2217 completes its travel stroke signifying that medicament delivery process has been completed.

More particularly, as shown in FIG. 30A, the elastomeric member 2217 includes a proximal end portion 2218 and is coupled to the distal member 2320 of the gas venting assembly 2310. In this manner, as described below, when the pressurized gas is conveyed into the medicament cavity 2139 (or “housing gas chamber”), the pressurized gas flows through the delivery control assembly 2430 and into a medicament container gas chamber located above the elastomeric member 2217 (i.e. bounded between the delivery control assembly 2430, elastomeric member 2217 and an interior of the medicament container body 2210). The pressure in the medicament container gas chamber exerts a force on the proximal surface 2218 to move the elastomeric member 2217 within the container body 2210 (i.e., to expel the medicament therefrom). Further, because the elastomeric member 2217 is coupled to the gas venting assembly 2310, movement of the elastomeric member 2217 within the container body 2210 produces movement of at least a portion of the distal member 2320. Similarly stated, when the elastomeric member 2217 is exposed to a force (e.g., produced by the pressurized gas within the medicament body gas chamber 2440 acting directly on the proximal surface 2218), movement of the elastomeric member 2217 exerts a force on the distal member 2320. Specifically, distal movement of the elastomeric member 2217 can produce a tensile force on the distal member 2320 causing the distal member 2320 to be moved distally out of housing.

As shown in FIG. 30A, the distal member 2320 can be coupled to the elastomeric member 2217 in any suitable manner. For example, as shown, the proximal surface 2218 receives and/or couples to a protrusion 2323 of the distal member 2320 of the gas venting assembly 2310. In some embodiments, the distal member 2320 includes a threaded portion and proximal end portion 2218 includes a corresponding threaded portion to receive the distal member 2320. In some embodiments, the threaded portion of the distal member 2320 is a self-tapping threaded portion. In other embodiments, the distal member 2320 can be threadedly coupled to the elastomeric member 2217. In yet other embodiments, the distal member 2320 can be bonded to the elastomeric member 2217 via an adhesive, a weld process, or the like.

The elastomeric member 2217 can be of any design or formulation suitable for contact with the medicament. For example, the elastomeric member 2217 can be formulated to minimize any reduction in the efficacy of the medicament that may result from contact (either direct or indirect) between the elastomeric member 2217 and the medicament. For example, in some embodiments, the elastomeric member 2217 can be formulated to minimize any leaching or out-gassing of compositions that may have an undesired effect on the medicament. In other embodiments, the elastomeric member 2217 can be formulated to maintain its chemical stability, flexibility and/or sealing properties when in contact (either direct or indirect) with the medicament over a long period of time (e.g., for up to six months, one year, two years, five years or longer).

In some embodiments, the elastomeric member 2217 can be constructed from multiple different materials. For example, in some embodiments, at least a portion of the elastomeric member 2217 can be coated. Such coatings can include, for example, polydimethylsiloxane. In some embodiments, at least a portion of the elastomeric member 2217 can be coated with polydimethylsiloxane in an amount of between approximately 0.02 mg/cm² and approximately 0.80 mg/cm².

The medicament container assembly 2200 can have any suitable size (e.g., length and/or diameter) and can contain any suitable volume of the medicament. The medicament container assembly 1200 can be constructed from any suitable materials including but is not limited to, glass, cyclic olefin copolymer (COC), and cyclic olefin polymers (COP) Moreover, although the medicament container assembly 2200 is shown as being a cartridge, in other embodiments, the medicament container assembly 2200 (and any of the medicament container assemblies described herein) can be a prefilled (or prefillable) syringe, such as those manufactured by Becton Dickinson, Gerresheimer, Ompi Pharma or others. For example, in some embodiments, the medicament container assembly 2200 (and any of the medicament container assemblies described herein) can be a Becton Dickinson “BD Hypak Physiolis” prefillable syringe containing any of the medicaments described herein. The medical injector 2000 can be configured to inject any suitable dosage such as, for example, a dose of up to 4 mL of any of the medicaments described herein. In other embodiments, the medical injector 2000 can be configured to inject a dose of up to 2 mL, 3 mL, 4 mL, 5 mL, 6 mL, 7 mL, 8 mL, 9 mL, 10 mL, or more of any of the medicaments described herein. The container body 2210 can be constructed from glass, and can be fitted and/or coupled to any suitable needle. For example, in some embodiments, the container body 2210 can be coupled (via any of the needle assemblies describing herein) to a needle having any suitable size.

As shown in FIG. 28, the medicament delivery mechanism 2300 includes a gas vent assembly 2310 (also referred to as an expandable assembly), but does not rely on a piston or rigid member to move the elastomeric member 2217 within the container body 2210 to inject the medicament. Rather, the elastomeric member 2217 is moved by the force produced by the pressurized gas within the gas chamber (or medicament cavity 2139). Accordingly, the stroke length and/or the dosage amount can be set by the expanded length of the gas vent assembly 2310. In this manner, the length of the medicament container assembly 2200 and the length of the gas vent assembly 2310 can be configured such the desired dosage amount is delivered. Moreover, because the gas vent assembly 2310 moves from a collapsed to an expanded configuration, the medicament delivery mechanism 2300 can fit within the same housing 2100 regardless of the fill volume, the delivery volume and/or the ratio of the fill volume to the delivery volume. In this manner, the same housing and production tooling can be used to produce devices having various dosages of the medicament. For example, in a first embodiment (e.g., having a fill volume to delivery volume ratio of 0.4), the medicament container has a first length and the movable member of the gas vent assembly has a first length. In a second embodiment (e.g., having a fill volume to delivery volume ratio of 0.6), the medicament container has a second length shorter than the first length, and the movable member of the gas vent assembly has a second length longer than the first length. In this manner, the stroke of the device of the second embodiment is longer than that of the device of the first embodiment, thereby allowing a greater dosage. The medicament container of the device of the second embodiment, however, is shorter than the medicament container of the device of the first embodiment, thereby allowing the components of both embodiments to be disposed within the same housing and/or a housing having the same length.

In some embodiments, the medical injector 2000 is configured such that a ratio of the housing length L_{H} to the container length L_{C} is less than about 1.5. In other embodiments, the medical injector 2000 is configured such that a ratio of the housing length L_{H} to the container length L_{C} is less than about 1.25. In yet other embodiments, the medical injector 2000 is configured such that a ratio of the housing length L_{H} to the container length L_{C} is less than about 1.1.

In some embodiments, the medical injector 2000 is configured such that a ratio of the housing length L H to a sum of the container length L_{c}, the carrier distance, and the stroke is less than about 1.1. In other embodiments, the medical injector 2000 is configured such that a ratio of the housing length L_{H} to a sum of the container length L_{c}, the carrier distance, and the stroke is less than about 1.0. In yet other embodiments, the medical injector 2000 is configured such that a ratio of the housing length L_{H} to a sum of the container length L_{c}, the carrier distance, and the stroke is less than about 0.9.

As shown in FIGS. 27 and 28, the system actuator assembly 2500 (similar to the system actuator assembly 1500 described above) includes an actuator input member 2510, a release member 2550, and an actuator spring 2576. The release member 2550 has a first (i.e., proximal) end portion 2551 and a second (i.e., distal) end portion 2552, and is movably disposed within the distal end portion of the gas container cavity 2151. The proximal end portion of the release member 2550 includes a sealing member 2574 and a puncturer 2575. The sealing member 2574 is configured to engage the sidewall of the housing 2100 defining the gas container cavity 2151 such that the proximal end portion of the gas container cavity 2151 is fluidically isolated from the distal end portion of the gas container cavity 2151. In this manner, when gas is released from the gas container 2410, the gas contained in the proximal end portion of the gas container cavity 2151 is unable to enter the distal end portion of the gas container cavity 2151. The puncturer 2575 of the release member 2550 is configured to contact and puncture a frangible seal 2413 on the gas container 2410 when the release member 2550 moves proximally within the gas container cavity 2151.

The distal end portion 2552 of the release member 2550 includes one or more extensions 2553. The extensions 2553 have projections that include tapered surfaces and engagement surfaces. Further, the extensions 2553 define an opening between the adjacent extensions 2553. The engagement surfaces of the extensions 2553 are configured to contact the release member contact surface 2146 of the housing 2100 and to pass through a release member aperture (not shown, but similar in construction as the release member aperture 1145 described herein) when the extensions 2553 are compressed inwards. In this manner, the engagement surfaces limit proximal movement of the release member 2550 in the normal, uncompressed state. The opening (not shown, but similar in construction as the opening 1154 described herein) defined by the extensions 2553 is configured to allow the extensions 2553 to flex and retract the engagement surfaces of the extensions 2553 inwards. An opening 2554 is defined between the extensions 2553. In some embodiments, a safety pin (not shown) can be inserted into the opening 2554 to prevent the extensions 2553 from moving, thereby disabling the release member 2550 until the safety pin is removed.

The tapered surfaces of the extensions 2553 are configured to contact corresponding tapered or conical surfaces (not shown) of the actuator input member 2510 (which is similar in construction as the actuator input member 1510 described herein) when the actuator input member 2510 is moved from a first position (i.e., released position or home position) to a second position (i.e., depressed position or active position). For example, as generally shown in FIGS. 27-29, as the actuator input member 2510 is depressed by an operator, the actuator input member 2510 rotates clockwise about support pin 2511. As the actuator input member 2510 is rotated, the tapered or conical surfaces of the actuator input member 2510 contact the extensions 2553. Continued movement of the actuator input member 2510 causes the extensions 2553 to flex inward until the engagement surfaces of the extensions 2553 are no longer in contact with the release member contact surface 2146 of the housing 2100. Force applied by the actuator spring 2576 on the proximal end portion 2551 against the housing 2100 causes the extensions 2553 to pass through the release member aperture (not shown) and the release member 2550 to move proximally within the gas container cavity 2151 towards the gas container 2410. In some embodiments, an opening is defined between the extensions 2553. In some embodiments, a safety pin (not shown) can be inserted into the opening to prevent the extensions 2553 from moving, thereby disabling the release member 2550 until the safety pin is removed.

The gas container 2410 includes a second (or distal) end portion 2411 and a first (or proximal) end portion 2412, and is configured to contain and/or produce a pressurized gas. The distal end portion 2411 of the gas container 2410 contains a frangible seal 2413 configured to break when the puncturer 2575 of the release member 2550 contacts the frangible seal 2413. The gas container retention member 2180 of the housing cap 2110 of the housing 2100 is configured to receive and/or retain the proximal end portion 2412 of the gas container 2410. Said another way, the position of the gas container 2410 within the gas container cavity 2151 is maintained by the gas container retention member 2180.

As shown in FIGS. 27 and 28, the length of the gas container retention member 2180 and the length of the release member 2550 collectively determine the distance between the puncturer 2575 and the frangible seal 2413 when the medical injector 2000 is in the storage configuration. Accordingly, this distance, which is the distance through which the puncturer 2575 travels when the medical injector 2000 is actuated via the actuator input member 2510, can be adjusted by changing the length of the gas container retention member 2180 and/or the length of the release member 2550. In some embodiments, the actuation time and/or the force exerted by the puncturer 2575 on the frangible seal 2413 can be adjusted by changing the distance between the puncturer 2575 and the frangible seal 2413.

As shown in FIGS. 27-29, the medicament delivery mechanism 2300 includes an insertion member 2360, a delivery control assembly 2430 (also referred to as a delivery control mechanism), a gas vent assembly 2310, and a needle assembly 2250. The insertion member 2360, gas vent assembly 2310, and a needle assembly 2250 are each movably disposed within the housing 2100. The insertion member 2360 is movable within the delivery mechanism cavity 2161 (see e.g., FIGS. 29 and 30). The gas vent assembly 2310 is movable within the gas passageway 2135 (see e.g., FIG. 30A). At least a portion of the needle assembly 2250 is rotatable within the delivery mechanism cavity 2161 to move a needle 2260 of the needle assembly 2250 from a first needle orientation (i.e., retracted orientation) to a second needle orientation (i.e., deployed orientation) (similar to the actuation shown in FIGS. 18 and 19).

The insertion member 2360 (which is similar to the insertion member 1360 described herein) includes a first (or proximal) end portion 2361, a second (or distal) end portion 2362, and defines a groove (see e.g., FIG. 27). The groove is configured to support an O-ring 2370. The O-ring is configured to engage the sidewall of the housing 2100 defining the delivery mechanism cavity 2161 such that the proximal end portion of the delivery mechanism cavity 2161 is fluidically isolated from the distal end portion of the delivery mechanism cavity 2161. In this manner, when the gas is released from the gas container 2410, the gas conveyed to the proximal end portion of the delivery mechanism cavity 2161 is unable to enter the distal end portion of the delivery mechanism cavity 2161. As described below, the proximal end portion 2361 of the insertion member 2360 includes a proximal surface 2376, which forms a portion of the boundary of the housing gas chamber (i.e., the portion of delivery mechanism cavity 2161).

As shown in FIG. 27, the distal end portion 2362 includes a contact portion 2364 for engaging and moving at least a portion of the needle assembly 2250. The contact portion 2364 includes a first guide channel (not shown) and a second guide channel 2366. Although the first guide channel and the second guide channel 2366 is depicted as extending along a linear path, in some embodiments, the first and second guide portions can define a curvilinear path to alter the deployment and retraction characteristics of the needle 2260. In some embodiments, the contact portion 2364 extends at an angle (i.e., diagonally) as it traverses from the distal end portion 2362 towards the proximal end portion 2361. Stated differently, the contact portion 2364 extends along an axis that is non-parallel with and non-perpendicular to a central axis of the insertion member 2360.

The needle assembly 2250 includes a needle coupling member 2251 with a first end portion 2252 and a second end portion 2253 (see e.g., FIG. 27) The needle assembly 2250 includes the receiving portion 2254 for receiving a portion of the medicament container, such as the neck 2213 of the medicament container assembly 2200. As shown, the receiving portion 2254 includes a puncturer 2259 to puncture a frangible seal 2219 at the distal end of the medicament container assembly 2200 upon insertion into the receiving portion 2254.

The receiving portion 2254 includes a central axis. The first end portion 2252 includes a pair of mounting bosses 2255 extending perpendicularly relative to the central axis of the receiving portion 2254. As shown in FIGS. 27 and 28, the pair of mounting bosses 2255 are rotatably supported by annular recesses 2171, 2172 of the housing. Stated differently, the pair of mounting bosses 2255 can be cylindrical protrusions extending from the first end portion 2252. The annular recess 2171 can be a blind hole formed in medicament cavity 2139 of the housing 2100 and the annular recess 2172 can be a blind hole formed in the delivery mechanism cavity 2161. The pair of mounting bosses 2255 define a rotational axis (A_{R}) about which the needle assembly 2250 rotates during operation. In some embodiments, a centerline of the pair of mounting bosses 2255 intersects a centerline extending from a distal end portion of the medicament container assembly 2200.

The second end portion 2253 of the needle coupling member 2251 includes a needle support portion 2256 for supporting the needle 2260. The needle support portion 2256 includes a central axis. The second end portion 2253 includes a pair of guide bosses 2257 extending perpendicularly relative to the central axis of the needle support portion 2256. In some embodiments, the pair of mounting bosses 2255 are parallel with the pair of guide bosses 2257. The pair of guide bosses 2257 are configured to engage and ride along the guide channels 2365, 2366 of the contact portion 2364. Stated in a different manner, the ramped nature of the contact portion 2364 causes the pair of guide bosses 2257 to move orthogonally relative to a longitudinal axis of the delivery mechanism cavity 2161.

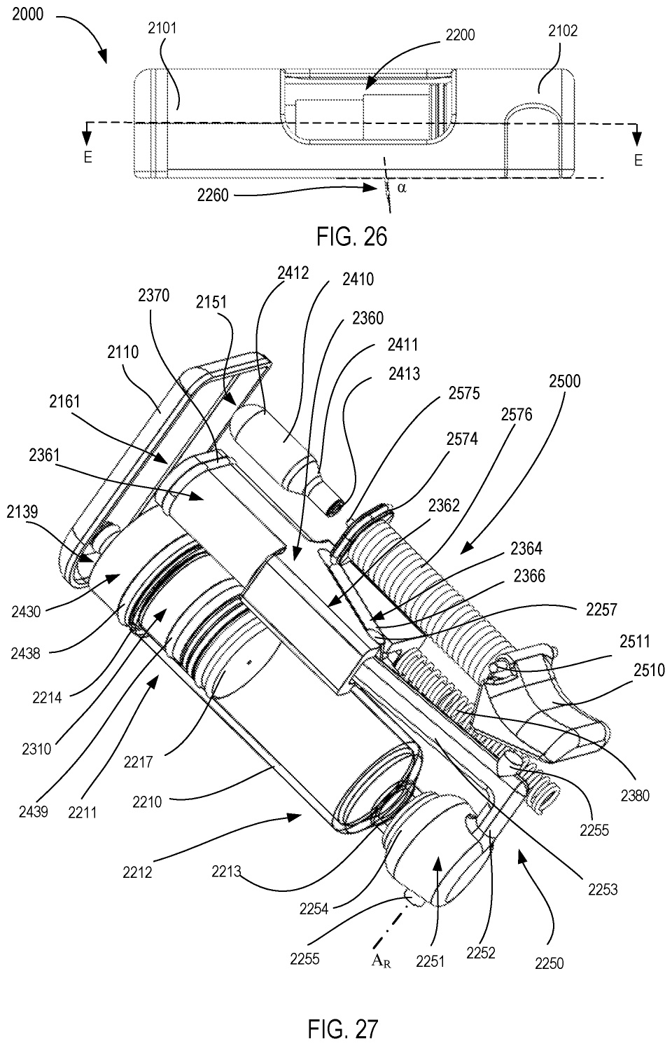

When the device is actuated, pressurized gas flows into the housing gas chamber and within the delivery mechanism cavity 2161. In this manner, the pressurized gas produces a force on the proximal surface 2376, which moves the insertion member 2360 distally within the housing 2100. When the pressurized gas produces a force on the proximal surface 2376, the force from the pressurized gas is high enough such that the insertion member 2360 overcomes a force applied by the retraction spring 2380. As a result, the insertion member 2360 moves distally within the delivery mechanism cavity 2161 of the housing 2100 (see e.g., FIGS. 29 and 30). As the insertion member 2360 moves distally, the first and second guide channels 2365, 2366 advances distally within the housing 2100 causing the guide bosses 2257 to be moved downward towards the bottom portion 2104 of the housing 2100. In turn, the needle assembly 2250 is rotated about the rotational axis (A_{R}). In some embodiments, the needle assembly 2250 is rotated about the rotational axis (A_{R}) by about 5 degrees to about 45 degrees while the needle 2260 is moved from the retracted orientation to the deployed orientation. In some embodiments, the needle assembly 2250 is rotated about the rotational axis (A_{R}) by about 10 degrees to about 30 degrees. In some embodiments, the needle 2260 extends at a non-orthogonal angle relative to a plane of the bottom portion 2104 of the housing 2100. In some embodiments, the needle 2260 extends at an angle (α) of between about 80 degree to about 88 degree relative to the plane of the bottom portion 1104 (see e.g., FIGS. 25 and 26). In some embodiments, the needle 2260 extends at an angle (α) of between about 75 degree to about 87 degree relative to the plane of the bottom portion 2104.

Concurrently with or after the insertion member 2360 has been moved distally, the gas pressure built up in the housing gas chamber at the proximal portion of the medicament cavity 2139 applies a force on the proximal surface 2439 of the delivery control assembly 2430. As shown in FIGS. 29 and 30, the delivery control assembly 2430 is coupled to the proximal end portion 2211 of the medicament container assembly 2200 and the medicament container assembly 2200 is movable within medicament cavity 2139. The force applied on the proximal surface 2439 of the delivery control assembly 2430 causes the medicament container assembly 2200 to move distally (toward the second end portion 2102 of the housing) until the distal end portion 2212 of the medicament container assembly 2200 contacts a shoulder portion 2106 of the housing 2100 (see e.g., FIGS. 29 and 30). In some embodiments, the shoulder portion 2106 is an annular ring extending radially inward from a side wall of the medicament cavity 2139. In some embodiments, a cushioning member or a spring member can be interposed between the distal end portion 2212 and the shoulder 2106 to control movement and/or impact as the medicament container assembly 2200 moves in the distal direction due to the force applied on the proximal surface 2439 of the delivery control assembly 2430.

As the medicament container assembly 2200 moves distally, the puncturer 2259 of the needle assembly 2250 punctures the frangible seal 2219 at the distal end portion 2212 of the medicament container assembly 2200 as it is inserted into the receiving portion 2254. The puncturer 2259 places an internal passage of the needle assembly 2250 in fluid communication with an internal volume of the medicament container body 2210. The internal passage is in fluid communication with the needle 2260. In other words, once the distal end portion 2212 of the medicament container assembly 2200 advances onto the puncturer 2259, thereby piercing the frangible seal 2219, the puncturer 2259 is operable to convey contents (e.g., medicament) within the medicament container body 2210 to the needle 2260.

Once the needle 2260 has been placed in the deployed orientation and the medicament container assembly 2200 has moved from a first position (proximal position) to a second position (distal position), gas pressure continues to build up within the housing gas chamber (e.g., in the proximal portions of the delivery mechanism cavity 2161 and medicament cavity 2139). As described herein, the delivery control assembly 2430 includes the first body portion 2431 and the second body portion 2432. As shown in FIG. 30A, the first body portion 2431 includes an external groove configured to retain an O-ring 2441. The O-ring 2441 rides along a side wall of the medicament cavity 2139 as the medicament container assembly 2200 and the delivery control assembly 2430 are moved in a distal direction in response to the force applied on the proximal surface 2439 of the delivery control assembly 2430. The O-ring 2441 prevents gas or fluid flow between the side wall of the medicament cavity 2139 and the delivery control assembly 2430.

The first body portion 2431 includes an internal shoulder portion configured to retain an O-ring 2442. The O-ring 2442 rides along an external surface the guide wall 2115 of the housing cap 2110 as the medicament container assembly 2200 and the delivery control assembly 2430 are moved in a distal direction in response to the force applied on the proximal surface 2439 of the delivery control assembly 2430. The O-ring 2442 prevents gas or fluid flow between the guide wall 2115 and the vent mechanism passageway 2436 of the delivery control assembly 2430.

The flow restriction retainer 2433 of the delivery control assembly 2430 supports at least a portion of a flow restriction member 2450. The flow restriction retainer 2433 includes a cylindrical inner surface and an end surface with a through-hole 2435 extending into an interior portion of the second body portion 2432. In this manner, the interior of the second body portion 2432 is in fluid communication with the flow restriction retainer 2433 of first body portion 2431. Although the through-hole 2435 is shown as being non-coaxial with a center of the flow restriction member 2450, in some embodiments, the through-hole 2435 can be coaxial with the flow restriction member 2450. In some embodiments, at least a portion of a flow restriction element 2452 overlaps with a portion of through-hole 2435. In some embodiments, at least 50% of the flow restriction element 2452 overlaps with the through-hole 2435.

As shown in FIG. 28, the flow restriction member 2450 includes a sleeve member 2451 and a flow restriction element 2452, and the flow restriction element 2452 is supported within the sleeve member 2451. In some embodiments, the sleeve member 2451 is a metal sleeve. In some embodiments, the metal sleeve is made of stainless steel or brass. In some embodiments, the flow restriction element 2452 is a porous material. In some embodiments, the porous material is sintered porous metal. In some embodiments, the flow restriction element 2452 is calibrated with nitrogen gas (N₂) at 30 psig (inlet side) to atmosphere (outlet side) at standard temperature and pressure to have a flow rate of between 0.5 to 3 standard cubic centimeter per minute (sccm). In some embodiments, the flow restriction element 2452 is calibrated with nitrogen gas (N₂) at 30 psig (inlet side) to atmosphere (outlet side) at standard temperature and pressure to have a flow rate of between about 0.75 and 1.5 standard cubic centimeter per minute (sccm). In some embodiments, the flow restriction element 2452 is calibrated with nitrogen gas (N₂) at 30 psig (inlet side) to atmosphere (outlet side) at standard temperature and pressure to have a flow rate of about 1 standard cubic centimeter per minute (sccm). As described herein, standard temperature is 60° F. (15.6° C.) and standard pressure is 14.696 psia (101.3 kPa).

In some embodiments, the compressed gas supplied by the gas container 2410 is an argon gas and the flow restriction element 2452 has a flow rate rating of about 0.75 and 1.5 sccm based on the nitrogen gas calibration described above. In some embodiments, the compressed gas supplied by the gas container 2410 is an argon gas and the flow restriction element 2452 has a flow rate rating of about 1 sccm based on the nitrogen gas calibration described above. In some embodiments, the compressed gas in the gas container 2410 has a molecular weight greater than the molecular weight of argon. For example, in some embodiments, the compressed gas supplied by the gas container 2410 is R134a (Tetrafluoroethane) and the flow restriction element 2452 has a flow rate rating of about 10 to 100 sccm based on the nitrogen gas calibration described above. In some embodiments, the compressed gas supplied by the gas container 2410 is R134a (Tetrafluoroethane) and the flow restriction element 2452 has a flow rate rating of about 20 to 40 sccm based on the nitrogen gas calibration described above.

In some embodiments, the flow rate of the medicament can be reduced to less than 0.2 mL/sec (or in some embodiments between 0.05 mL/sec and 0.01 mL/sec) using gas pressure that is initially supplied to medicament cavity 2139 and through the flow restriction member 2450. The lower injection forces and/or slower delivery (compared with pressures supplied directly from the medicament cavity 2139 to the elastomeric member) can produce laminar flow of the medicament through the needle, prevent shearing of high molecular weight compounds in the medicament, and/or reduce pain sensed by a patient particularly if the medicament being delivered is very high viscosity (e.g., greater than about 100 centipoise at room temperature). In some embodiments, a screen or mesh protective member can be provided on a proximal side of the flow restriction member 2450 to prevent any particulate or debris from clogging the flow restriction element 2452 during operation.

As shown in FIG. 30A, the second body portion 2432 is inserted within the proximal end portion 2211 of the medicament container body 2210. The second body portion 2432 includes an external groove configured to retain an O-ring 2443. The O-ring 2443 prevents gas or fluid flow from passing between the outer cylindrical surface of the second body portion 2432 and an internal wall of the medicament container assembly 2200. Stated differently, the O-ring 2443 seals the medicament container assembly 2200 from the housing gas chamber portion of the medicament cavity 2139.

The inner diameter of the cylindrical inner surface 2434 is less than an inner diameter of the second body portion 2432. The second body portion 2432 further includes a flange portion 2438 extending radially from an outer surface of the second body portion 2432. The flange portion 2438 is configured to mount onto the end surface 2214 of the medicament container body 2210. Although not shown, in some embodiments, one or more O-rings can be supported on the cylindrical inner surface 2434 to prevent pressurized gas from passing between the flow restriction member 2450 and the cylindrical inner surface 2434. In other words, the O-rings can be provided to prevent pressurized gas from bypassing around the flow restriction member 2450.

As described above, gas pressure is applied on the proximal surface 2376 of the insertion member 2360 to move the insertion member 2360 distally within the delivery mechanism cavity 2161 the housing 2100. Gas pressure is also applied on the proximal surface 2439 of the delivery control assembly 2430 to move the delivery control assembly 2430 and the medicament container assembly 2200 distally within the medicament cavity 2139 of the housing 2100. The distal movement of the insertion member 2360 causes the guide bosses 2257 to move along the first and second guide channels 2365, 2366 until the needle 2260 has been extended by a desired distance from the bottom portion 2104 of the housing 2100. In some embodiments, the delivery control assembly 2430 can permit gas to pass through the flow restriction member 2450 but not build enough pressure to move the elastomeric member 2217 until after the needle 2260 has been deployed and/or until the medicament container assembly 2200 has been seated onto the needle assembly 2250 and that the puncturer 2575 has been inserted into the frangible seal 2413. In some embodiments, the pressurized gas in the medicament cavity 2139 drops to about 90-100 psi at after the needle 2260 has been deployed. Although the medicament container body 2210 is shown as interfacing with the sidewalls of the medicament cavity 2139, in some embodiments, the medicament container assembly 2200 can include a medicament container carrier supporting the medicament container body 2210. The medicament container carrier is configured to travel within and contact the sidewalls of the medicament cavity 2139. In some embodiments, the medicament container carrier can contact the shoulder portion 2106 and reduce impact force transferred from the shoulder portion 2106 to the medicament container body 2210 as the medicament container assembly 2200 is moved to the distal position.

As shown in FIGS. 29-32, the gas from the medicament cavity 2139 passes through and is regulated by the flow restriction member 2450 to begin delivery of medicament from within the medicament container body 2210. The gas passing through the flow restriction member 2450 travels through the through-hole 2435 (see e.g., FIG. 30A). After passing through the through-hole 2435, the gas enters a medicament body gas chamber 2440 sealed between a distal side of the O-ring 2442 and a proximal side of the elastomeric member 2217. This causes the elastomeric member 2217 to move in the distal direction within the medicament container body 2210. Distal movement of the elastomeric member 2217 generates a pressure upon the medicament contained within the medicament container assembly 2200, thereby allowing at least a portion of the medicament to flow out of the medicament container assembly 2200 via the needle 2260. The medicament is delivered to a body of a user via the medicament delivery path defined by the medicament container assembly 2200, the internal passage of the needle assembly 2250, and the needle 2260.

Once the elastomeric member 2217 has completed its travel stroke to deliver a desired dose of medicament, the gas vent assembly 2310 operates to retract the needle 2260 back into the housing 2100 as detailed below. The gas vent assembly 2310 is configured to expand and/or change configurations during operation of the medical injector 2000, and selectively produces a pathway through which pressurized gas escapes the medicament cavity 2139 after delivery of the medicament. By releasing or removing the force from the gas passageway 2135, the insertion member 2360 and the retraction spring 2380 can move (i.e. rotate) the needle assembly 2250 from the second needle orientation (i.e., deployed orientation) back to the first needle orientation (i.e., retracted orientation), thereby retracting the needle 2260 back within the housing 2100. For example, as pressurized gas is released, the force of the retraction spring 2380 becomes greater than the gas pressure applied on the proximal surface 2376 of the insertion member 2360. As a result, the insertion member 2360 moves proximally within the delivery mechanism cavity 2161. As the insertion member 2360 moves proximally, the first and second guide channels 2365, 2366 also move proximally causing the pair of guide bosses 2257 to move upwards and away from the bottom portion 2104 of the housing 2100. In turn, the needle assembly 2250 is rotated about the rotational axis (A_{R}) and the needle 2260 is retracted back within the housing 2100. Although the retraction spring 2380 is shown as a coil spring acting in the proximal direction (i.e. push the insertion member 2360 proximally), in some embodiments, a wound spring can be coupled to insertion member 2360 to pull the insertion member 2360 in the proximal direction.

Notably, the gas vent assembly 2310 does not exert a distal force on the elastomeric member 2217, but rather, is carried distally by the elastomeric member 2217 during delivery of the medicament. Thus, this arrangement is considered a “pistonless” delivery system, because the force for insertion and medicament delivery is provided via the pressurized gas acting either directly upon the medicament container assembly 2200 (e.g., the proximal surface 2218 of the elastomeric member 2217), the delivery control assembly 2430 (e.g., the first body portion 2431 and the second body portion 2432 of the delivery control mechanism extending out of the medicament container body 2210), and/or the insertion member 2360 (e.g., the proximal surface 2376 of the insertion member 2360), or indirectly through gas pressure supplied from the medicament cavity 2139 through the delivery control assembly 2430 via the flow restriction member 2450 as described herein.

As shown in FIGS. 30-33, the gas vent assembly 2310 includes the first (or distal) member 2320, the second (or intermediate) member 2330 and the third (or proximal) member 2340. The proximal member 2340 includes a base portion 2341 and a valve portion 2345 extending proximally from the base portion 2341. The valve portion 2345 includes a first groove for retaining a first O-ring 2342 and a second groove for retaining a second O-ring 2343. The first O-ring 2342 and the second O-ring 2344 are configured to ride on an internal surface of the guide wall 2115. As shown in FIGS. 31, 32 and 32A, distal movement of the valve portion 2345 as the third member 2340 is pulled distally causes the first O-ring 2342 to ride over and unseal a vent port 2113, thereby placing the gas passageway 2135 in fluid communication with an external environment. This causes the pressurized gas from within the medicament cavity 2139 to escape out to the external environment. In other words, once the vent port 2113 is unsealed, pressurized gas from within the medicament cavity 2139 and the delivery mechanism cavity 2161 exits through the vent opening 2112 via the vent port 2113 (see e.g., FIG. 32A) allowing the needle assembly 2250 to rotate and the needle 2260 to retract.

The first member 2320, the second member 2330 and the third member 2340 are nested together such that the gas vent assembly 2310 can be transitioned from the collapsed configuration (FIGS. 28, 29, and 30) to an expanded configuration (FIGS. 31 and 32, just prior to complete delivery of medicament), and a series of partially expanded configurations therebetween. The gas vent assembly 2310 reaches the expanded configuration just prior to a complete dose of medicament being delivered (see e.g., FIG. 31). Once the gas vent assembly 2310 has been placed in the expanded configuration, the elastomeric member 2217 continues to travel a final distance to deliver the remaining amount from the complete dose, which in turn pulls on the valve portion 2345 to at least partially unseat it from the vent opening 2112. Stated in a different manner, the length of the gas vent assembly 2310 in the expanded configuration is selected to expand and reach the expanded configuration before the end of travel of the elastomeric member 2217. When the gas vent assembly 2310 is in the expanded configuration and continues to travel with the elastomeric member 2217 the final distance to finish delivery of the complete dose (FIGS. 31 and 32, 32A, after delivery of the medicament is complete), the vent opening 2112, the first O-ring 2342 and the vent port 2113 collectively allow the pressurized gas from the housing gas chamber of the medicament cavity 2139 to escape the medicament cavity 2139, such that needle retraction can occur.

Certain aspects of the gas vent assembly 2310 can be similar to or substantially the same to the gas vent assemblies described in International Application PCT/US2019/068750 entitled, “DEVICES AND METHODS FOR DELIVERY OF SUBSTANCES WITHIN A PREFILLED SYRINGE,” filed on Dec. 27, 2019, and International Application PCT/US2020/045467 entitled. “DEVICES AND METHODS FOR DELIVERY OF SUBSTANCES WITHIN A PREFILLED SYRINGE,” filed on Aug. 7, 2020, the disclosures of which are incorporated herein by reference in its entirety. For example, although the gas vent assembly 2310 is shown as having three nested tubular members, the gas vent assembly 2310 (and any gas vent assembly described herein) may include other nested configurations and/or may be provided with a flexible element that can be pulled and expanded (e.g., wire, filament, cord, ribbon, or the like).

The first member 2320 includes a proximal end portion and a distal end portion. The protrusion 2323 at the distal end portion of the first member 2320 is configured to matingly engage the elastomeric member 2217. In this manner, movement of the elastomeric member 2217 distally causes movement of first member 2320 distally. In some embodiments, the protrusion 2323 is a threaded portion that matingly engages the elastomeric member 2217. The proximal end portion of the first member 2320 includes an inwardly extending lip configured to engage a corresponding outwardly extending lip at the distal end portion of the second member 2330. The proximal end portion of the second member 2330 includes an inwardly extending lip configured to engage a corresponding outwardly extending lip at the distal end portion of the third member 2340. As the elastomeric member 4217 is moved distally during operation, the first member 2320 is moved distally away from the second and third member 2330, 2340, and the second member 2330 is moved distally away from the third member 2340. Stated differently, the first member 2320, the second member 2330, and the third member 2340 transition from a collapsed configuration to an expanded configuration by sliding apart from one another.

The gas vent assembly 2310 and the elastomeric member 2217 provide a visual indication of the status of the injection. As shown in FIG. 24, the status indicator aperture 2130 has a sufficient length to allow viewing substantially all of the medicament container body 2210. Thus, as the injection event proceeds, the members of the gas vent assembly 2310 (which are expanded within the medicament container body 2210) are visible via the status indicator aperture 2130. In some embodiments, the members of the gas vent assembly 2310 can include markings, different colors, or other indicium to indicate the ongoing status of the injection (e.g., “25 percent complete,” “50 percent complete”).

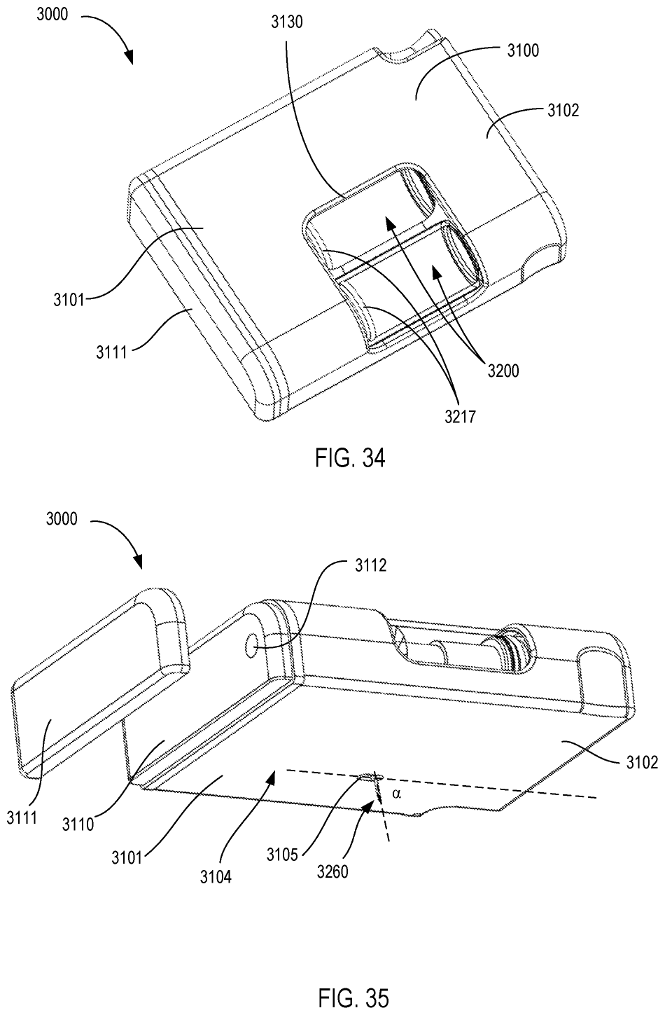

Although the medical injector 2000 is shown as including a single medicament container assembly 2200, in other embodiments, a medical injector can include two or more medicament container assemblies to deliver a large dose (e.g., >10 mL dose) of medicament, deliver two separate formulations of medicament, or deliver a mixed formulation of two medicament. For example, FIGS. 34-44 show a medical injector 3000 (also referred to as a medicament delivery device) with a housing 3100. The corresponding components in the medical injector 3000 that are similar to those provided in the medical injector 2000 are identified by the same references numerals. For example, the medical injector 3000 uses two of the medicament container assemblies 2200 of the type described above for the medical injector 2000. Thus, these assemblies are identified below as medicament container assemblies 2200 and are not described in detail with reference to the medical injector 3000.

The medical injector 3000 includes the housing 3100, a system actuation assembly 2500, a medicament delivery mechanism 3300, two medicament container assemblies 2200, and two gas vent assemblies 2310, The housing 3100 includes a first (i.e., proximal) end portion 3101 and a second (i.e., distal) end portion 3102. The housing 3100 includes a top portion 3103 extending between the first end portion 3101 and the second end portion 3102. The top portion 3103 defines a status indicator aperture 3130.

The status indicator aperture 3130 can allow a patient to monitor the status and/or contents of the medicament container assembly 3200, a position of the elastomeric members 2217 within the two medicament containers 2200, and/or and the medicament contained within the housing 3100. For example, by visually inspecting the status indicator aperture 3130, a patient can determine whether the medicament container assemblies 2200 contain a medicament and/or whether the medicament has been dispensed. In some embodiments, the status indicator aperture 3130 includes a length to display an entire, or substantially the entire, stroke length of the elastomeric members 2217 during operation. Although a single status indicator aperture 3130 is shown, in some embodiments, the housing 3100 can include one or more status indicator apertures associated with each of the medicament container assemblies 2200.

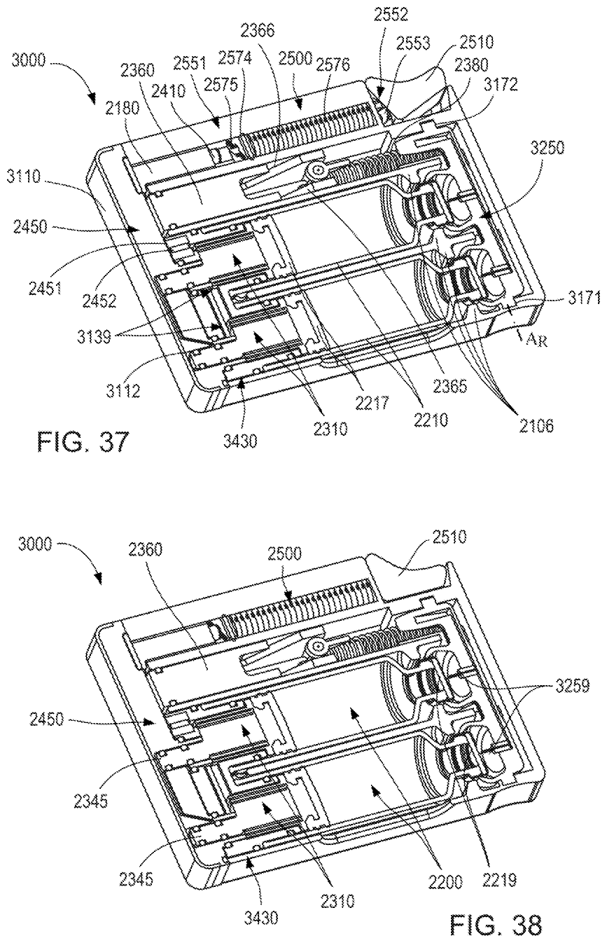

The housing 3100 defines two medicament cavities 3139, a gas container cavity 2151, and a delivery mechanism cavity 2161 (see e.g., FIG. 37). The gas container cavity 2151 is configured to receive the gas container 2410 (which can be the same as gas container 1410 or any other gas container described herein) and a portion of the system actuator assembly 2500. The proximal end portion of the gas container cavity 2151 is configured to support the gas container retention member 2180 (which can be the same as gas container retention member 1180, or any other gas container retention member described herein). The gas container cavity 2151 is in fluid communication with the delivery mechanism cavity 3161 and the two medicament cavities 3139 via a gas passageway 3135 defined in the housing 3100. As shown in FIG. 37, the distal portion of each of the medicament cavities 3139 includes a shoulder portion 3106 with a contact surface. The contact surface of the shoulder portion 3106 is configured to limit distal movement of the respective medicament container body 2210 similar to the shoulder portion 2106 in the medical injector 2000 described above.

Each of the two medicament cavities 3139 is configured to receive the medicament container assembly 2200 and at least a portion of the medicament delivery mechanism 3300. The medicament delivery mechanism 3300 includes an insertion member 2360 and a needle assembly 3250. Both of the medicament cavities 3139 are in fluid communication with the gas container cavity 2151, the delivery mechanism cavity 3161, and a vent opening 3112.

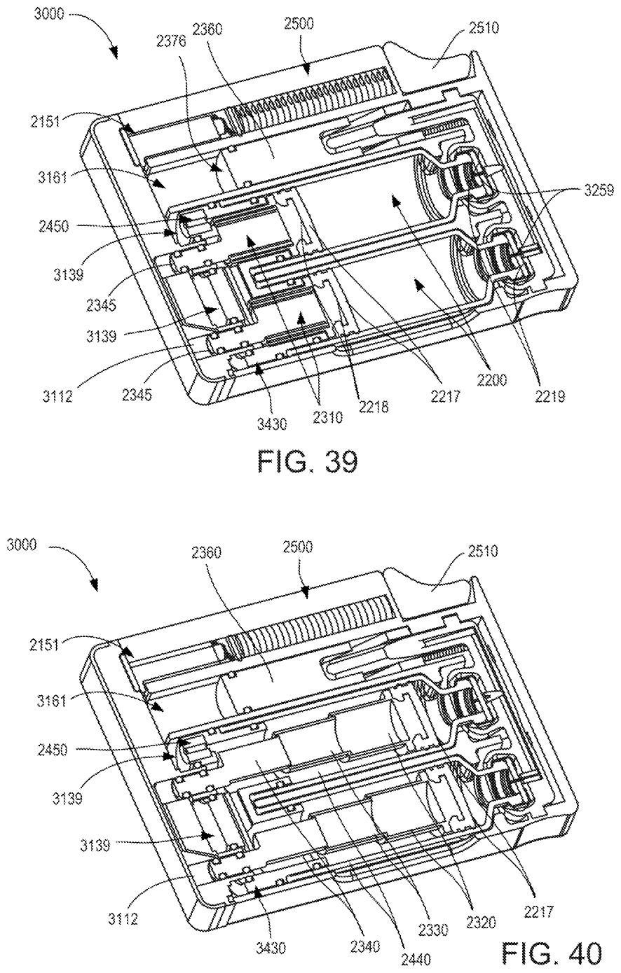

The first (i.e., proximal) end portion 3101 of the housing 3100 includes a housing cap 3110 and a cap cover 3111 (see e.g., FIG. 35). A gas container retention member 2180 is configured to be inserted into the gas container cavity 2151 to retain a gas container 2410 that contains a pressurized gas (see e.g., FIG. 37). The housing cap 3110 is configured to retain the gas container retention member 2180 within the gas container cavity 2151. When the medical injector 3000 is actuated, pressurized gas from the gas container 2410 is conveyed from the gas container cavity 2151 to the delivery mechanism cavity 3161 and to the two medicament cavity 3139 via the gas passageway 3135 of the housing 3100 (see e.g., FIGS. 38 and 39). Said another way, the gas passageway 3135 places the gas container cavity 2151 in fluid communication with the delivery mechanism cavity 3161 and the two medicament cavity 3139. Thus, the pressurized gas from the gas container 2410 is conveyed from the gas container cavity 2151 to the delivery mechanism cavity 3161 to move the insertion member 2360 and the needle assembly 3250. As will be described in greater detail below, the pressurized gas from the gas container 2410 is conveyed from the gas container cavity 2151 to a proximal portion of each of the two medicament cavities 3139 to actuate the two medicament container assemblies 2200. The proximal portion of each of the two medicament cavities 2139 also serve as a pressurized gas reservoir used to inject the medicament, as described herein.

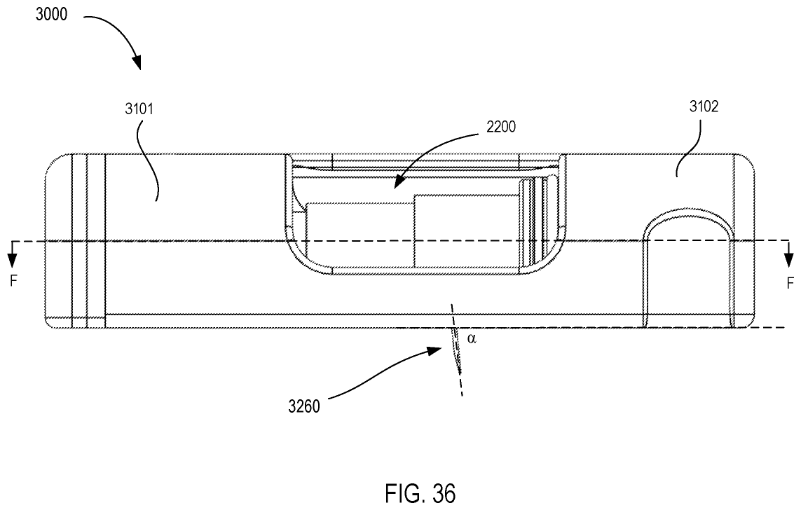

The insertion member 2360 of the medical injector 3000 is substantially the same and works in the same manner as the insertion member 2360 of the medical injector 2000. As the insertion member 2360 advances distally due to a force applied on the proximal surface 2376 by the pressurized gas, the needle assembly 3250 is rotated about the rotational axis (A_{R}) (see e.g., FIG. 37). In some embodiments, the needle assembly 3250 is rotated about the rotational axis (A_{R}) by about 5 degrees to about 45 degrees while the needle 3260 is moved from the retracted orientation to the deployed orientation. In some embodiments, the needle assembly 3250 is rotated about the rotational axis (A_{R}) by about 10 degrees to about 30 degrees. In some embodiments, the needle 3260 extends at a non-orthogonal angle relative to a plane of the bottom portion 3104 of the housing 3100. In some embodiments, the needle 3260 extends at an angle (α) of between about 80 degree to about 88 degree relative to the plane of the bottom portion 3104 (see e.g., FIGS. 35 and 36). In some embodiments, the bottom portion 3104 may be provided with an adhesive patch or material to further secure the bottom portion 3104 of the housing 3100 to the body surface of a patient. In some embodiments, a cover or a guard (not shown) may be provided over the needle aperture 3105 to prevent ingress of foreign matter into the housing 1100 through the needle aperture 3105, to maintain needle sterility, and/or to prevent accidental needle prick. In some embodiments, the medical injector 3000 (or any of the other medical injectors described herein) can be secured to the body surface of the patient with a bandage, strap, sleeve, band, or the like. In this manner, the medical injector 3000 can be wrap around a patient's arm, leg, or abdomen securely such that the patient can move about more freely without fear of the medical injector 3000 falling off.

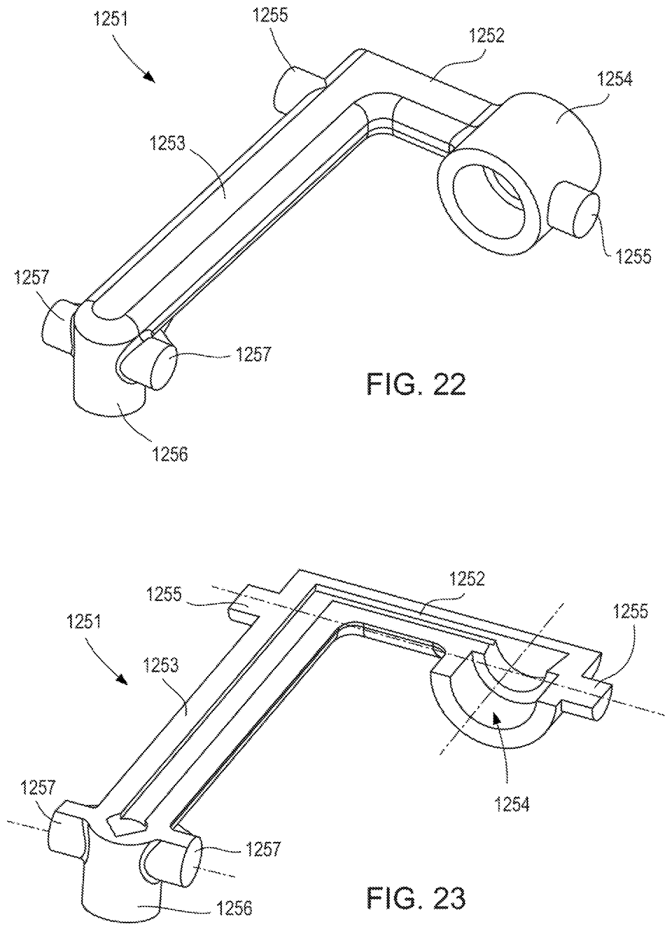

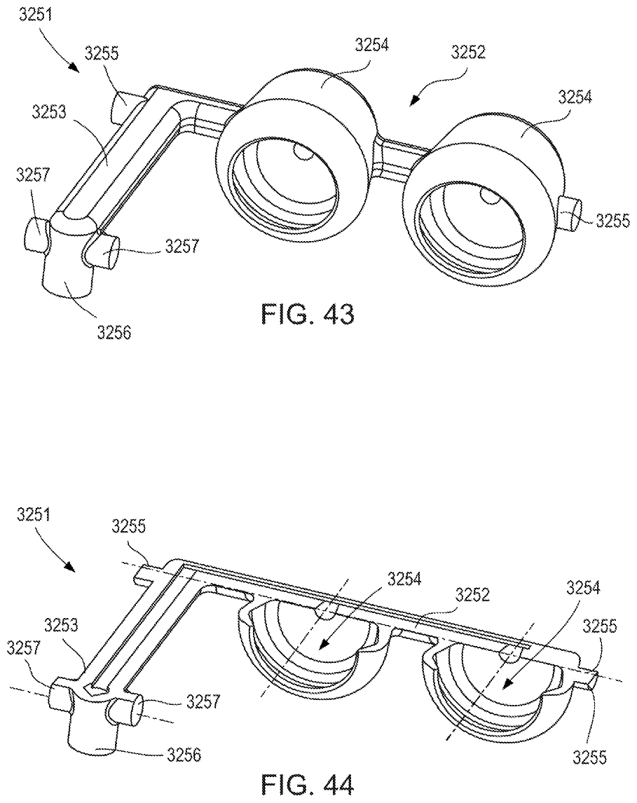

Comparing the needle assembly 3250 of the medical injector 3000 with the needle assemblies 1250, 2250, the needle assembly 3250 includes two receiving portions 3254 for receiving distal ends of the two medicament container assemblies 2200 (compare FIGS. 22-23 with FIGS. 43-44). The needle assembly 3250 includes a needle coupling member 3251 with a first end portion 3252 and a second end portion 3253. The needle assembly 3250 includes two receiving portions 3254, each configured to receive a portion of the medicament container, such as the neck 2213 of the medicament container assembly 2200. As shown, the receiving portions 3254 each include a puncturer 3259 to puncture a frangible seal 2219 at the distal end of the medicament container assemblies 2200 upon insertion into the receiving portion 3254.

The receiving portions 3254 each include a central axis (see e.g., FIG. 44). The first end portion 3252 includes a pair of mounting bosses 3255 extending perpendicularly relative to the central axes of the receiving portions 3254. The pair of mounting bosses 3255 are rotatably supported by annular recesses 3171, 3172 of the housing 3100 (see e.g., FIG. 37). Stated differently, the pair of mounting bosses 3255 can be cylindrical protrusions extending from the first end portion 3252. The annular recess 3171 can be a blind hole formed in one of the medicament cavities 3139 of the housing 3100 and the annular recess 2172 can be a blind hole formed in the delivery mechanism cavity 3161. The pair of mounting bosses 3255 define a rotational axis (A_{R}) about which the needle assembly 3250 rotates during operation.

The second end portion 3253 of the needle coupling member 3251 includes a needle support portion 3256 for supporting the needle 3260. The needle support portion 3256 includes a central axis. The second end portion 3253 includes a pair of guide bosses 3257 extending perpendicularly relative to the central axis of the needle support portion 3256. In some embodiments, the pair of mounting bosses 3255 are parallel with the pair of guide bosses 3257. The pair of guide bosses 3257 are configured to engage and ride along the guide channels 2365, 2366 of the contact portion 2364. Stated in a different manner, the ramped nature of the contact portion 2364 causes the pair of guide bosses 3257 to move orthogonally relative to a longitudinal axis of the delivery mechanism cavity 3161.

The housing cap 3110 includes a vent opening 3112 that can be selectively placed in fluid communication with the gas passageway 3135 within the housing 3100. The housing cap 3110 also includes a cap cover 3111 coupled to a proximal end portion of the housing cap 3110 while retaining a gap between the proximal end portion of the housing cap 3110 and the cap cover 3111. The cap cover 3111 prevents the vent opening 3112 from direct external contact and prevents clogging from external debris. The vent opening 3112 provides the passageway through which pressurized gas is conveyed from gas passageway 3135 (including from within the medicament cavities 3139 and the delivery mechanism cavity 3161) to a volume outside of the medical injector 3000. The vent opening 3112 and the gap between the housing cap 3110 and the cap cover 3111 allows pressurized gas from within the two medicament cavities 3139 to escape out to the volume outside the medical injector 3000. In this manner, the force produced by the pressurized gas on the medicament delivery mechanism 3300 and/or the two medicament container assemblies 3200 (via the delivery control assembly 3430) can be reduced to allow needle retraction after the injection is completed.

Although the vent opening 3112 is shown as being defined by the housing cap 3110, and being in a proximal surface thereof, in other embodiments, the vent opening 3112 (and any of the vent openings described herein) can be defined within any suitable portion of the housing cap or side wall. For example, in some embodiments, the vent opening 3112 (and any of the vent openings described herein) can be defined by the housing cap 3110 but can have a centerline that is nonparallel to a longitudinal axis of the medical injector 3000. Said another way, in some embodiments, the vent opening 3112 (and any of the vent openings described herein) can open towards a side of the medical injector, rather than opening towards the proximal end, as shown. In other embodiments, the vent opening 3112 (and any of the vent openings described herein) can be defined by any wall and/or surface of the housing 3100.

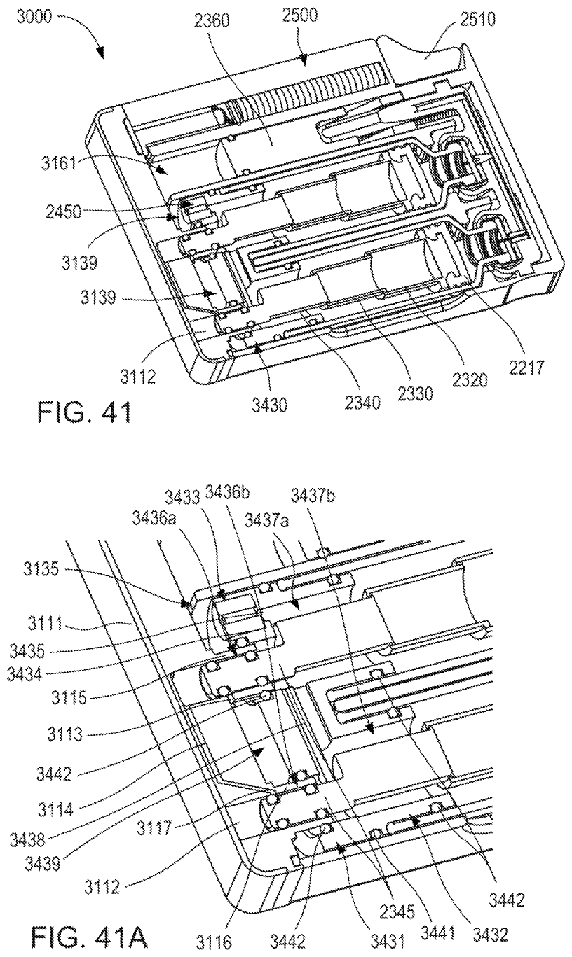

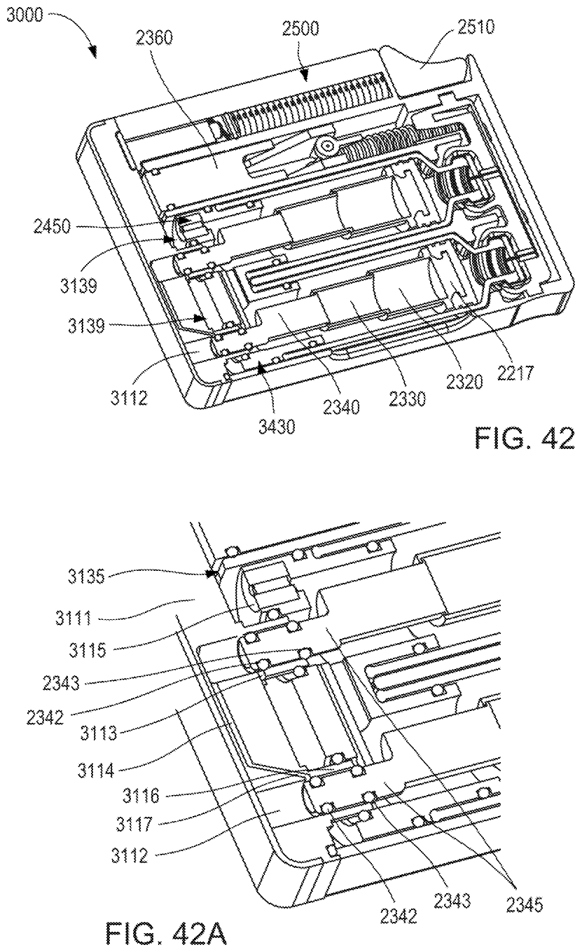

As shown in FIGS. 41A and 42A, the housing cap 3110 includes a first guide wall 3115 within which a first guide member of the first of the two gas vent assemblies 2310 moves to selectively place the vent inlet port 3113 in fluid communication with a vent passageway 3114. The housing cap 3110 further includes a second guide wall 3116 within which a second guide member of the second of the two gas vent assemblies 2310 moves to selectively place the vent outlet port 3117 in fluid communication with the vent passageway 3114 and in fluid communication with the vent opening 3112. In this manner, the gas passageway 3135 is only placed in fluid communication with an external environment when medicament has been fully dispensed from both medicament container assemblies 2200 and both valve portions 2345 have been moved to its most distal position (i.e., valve open position).

Specifically, the guide wall 3115 defines an inner cylindrical wall surface within which a valve portion 2345 of the first member 2340 (see e.g., FIGS. 38, 39, 41A, and 42A) slides during operation. The guide wall 3115 defines an outer cylindrical wall about which a guide surface of the delivery control assembly 3430 slides during actuation of the two medicament container assemblies 2200. The guide wall 3116 defines an inner cylindrical wall surface within which a valve portion 2345 of the second member 2340 slides during operation. The guide wall 3116 defines an outer cylindrical wall about which a guide surface of the delivery control assembly 3430 slides during actuation of the two medicament container assemblies 2200.

Similar to the medical injector 2000, once the needle 3260 of the medical injector has been placed in the deployed orientation, the two medicament container assembly 2200 assemblies moved from a first position (proximal position) to a second position (distal position) (see e.g., FIG. 39). In particular, pressurized gas applies a force on a proximal surface of the 3439 of the delivery control assembly 3430. Because the delivery control assembly 3430 is coupled to the first end portion 2211 of each of the two medicament container assemblies 2200, and each of the two medicament container assemblies 2200 are movable within their respective medicament cavities 3139, distal movement of the delivery control assembly 3430 causes the two medicament container assemblies 2200 to advance distally (toward the second end portion 3102 of the housing) and be inserted into the two receiving portions 3254. In some embodiments, similar to the medical injector 2000, as the two medicament container assemblies 2200 are inserted into the needle assembly 3250, the puncturers 3259 are inserted into the frangible seals 2219 of the two medicament container assemblies 2200 to place the medicament container bodies 2210 in fluid communication with the needle 3260. Although the needle assembly 3250 is shown as having one needle 3260 coupled to the two medicament container assemblies 2200, in some embodiments, the needle assembly 3250 may include two needles to increase delivery rate to the patient while preventing shearing of the medicament or therapeutic substance (i.e., medicament or substances with high molecular weight compounds). In some embodiments, the medical injector 3000 can include two needle assemblies (similar to needle assembly 2250), each of which is attached to one of the two medicament container assemblies 2200. In this manner, medicament from within each of the container assemblies can be dispensed independently through its own needle.

Referring to FIG. 41A, the delivery control assembly 3430 (also referred to as a delivery control mechanism) includes a first body portion 3431 and a second body portion 3432. The first body portion 3431 includes a flow restriction retainer 3433, a first vent mechanism passageway 3436a, and a second vent mechanism passageway 3436b. The flow restriction retainer 3433 includes a cylindrical inner surface 3434 and an end surface with a through-hole 3435 extending into an interior portion of the second body portion 3432. The flow restriction retainer 3433 is configured to support at least a portion of a flow restriction member 2450. The second body portion 3432 includes a first housing portion 3437a configured to house at least a portion of a first gas vent assembly (e.g., gas vent assembly 2310) and includes a second housing portion 3437b configured to house at least a portion of a second gas vent assembly (e.g., gas vent assembly 2310). The first housing portion 3437a and the second housing portion 3437b each include a cylindrical inner surface. Although the cylindrical inner surface 3434, gas vent passageways 3436a, 3436b, and the cylindrical inner surface of the first and second housing portions 3437a, 3437b are shown as having a circular cross-section, one or more of the cylindrical inner surface 3434, gas vent passageway 3436, the cylindrical inner surface of the first and second housing portions 3437a, 3437b can be configured to have an oval, elliptical, or other rounded cross-sections. Each of the gas vent assemblies 2310 associated with the two medicament container assemblies 2200 includes a first (or distal member) member 2320, a second (or intermediate) member 2330, and a third (or proximal) member 2340. These components are nested together such that each of the gas vent assemblies 2310 can be transitioned from a collapsed configuration (see e.g., FIGS. 37-39) to an expanded configuration (see e.g., FIGS. 40-42). The gas vent assemblies 2310 of the medical injector 3000 operates in the same manner as the gas vent assembly 2310 of the medical injector 2000.

The first body portion 3431 includes an external groove configured to retain an O-ring 3441. The O-ring 3441 rides along a side wall of the medicament cavity 2139 as the medicament container assembly 2200 and the delivery control assembly 3430 are moved in a distal direction in response to the force applied on the proximal surface 3439 of the delivery control assembly 3430. The O-ring 3441 prevents gas or fluid flow between the side wall of the medicament cavities 3151 and the delivery control assembly 3430.

The first body portion 3431 includes internal shoulder portions configured to retain an O-ring 3442. The O-ring 3442 rides along external surfaces of the guide walls 3115, 3116 of the housing cap 3110 as the medicament container assemblies 2200 and the delivery control assembly 2430 are moved in a distal direction in response to the force applied on the proximal surface 3439 of the delivery control assembly 3430. The O-ring 3442 prevents gas or fluid flow between the guide walls 3115, 3116 and the vent mechanism passageway 3436 of the delivery control assembly 3430.

When gas pressure is applied on the proximal surface 2376 of the insertion member 2360, the insertion member 2360 is moved distally within the delivery mechanism cavity 3161 of the housing 3100. The distal movement of the insertion member 2360 causes the guide bosses 3257 to move along the first and second guide channels 2365, 2366 until the needle 3260 has been extended by a desired distance from the bottom portion 3104 of the housing 3100. Gas pressure is also applied on the proximal surface 3439 of the delivery control assembly 3430 to move the delivery control assembly 3430 and the two medicament container assemblies 2200 distally within the medicament cavities 3139 of the housing 3100. In some embodiments, the delivery control assembly 3430 can permit gas to pass through the flow restriction member 2450 but not build enough pressure to move the elastomeric members 2217 until after the needle 3260 has been deployed and/or until the two medicament container assemblies 2200 have been seated onto the needle assembly 3250. In some embodiments, the pressurized gas in the medicament cavity 3139 drops to about 90-100 psi after the needle 3260 has been deployed.

As shown in FIGS. 39 and 40, the gas from the medicament cavity 3139 passes through and is regulated by the flow restriction member 2450 to begin delivery of medicament from within the two medicament container bodies 2210. The gas passing through the flow restriction member 2450 travels through the through-hole 3435. After passing through the through-hole 3435, the gas enters into one of the medicament body gas chambers 2440 sealed between a distal side of the O-ring 3442 and a proximal side of one of the elastomeric members 2217. This causes both of the elastomeric members 2217 to move in the distal direction within their respective medicament container bodies 2210. Distal movement of the elastomeric members 2217 generate pressure upon the medicament contained within each of the medicament container assemblies 2200, thereby allowing at least a portion of the medicament to flow out of each of the medicament container assemblies 2200 via the needle 3260. The medicament is delivered to a body of a user via the medicament delivery path defined by the medicament container assemblies 2200, the internal passage of the needle assembly 3250, and the needle 3260.

In some embodiments, the same medicament is provided in both medicament container assemblies 2200 and medicament is supplied to the needle assembly 3250 at the same delivery rate. In some embodiments, a first medicament is provided in a first medicament container assembly and a second medicament is provided in a second medicament container assembly. The first medicament has a greater viscosity than the second medicament. The second medicament is supplied to the needle assembly 3250 at a faster delivery rate than the first medicament is supplied to the needle assembly 3250. In some embodiment, an additional flow restriction member can be provided in the passageway 3438 extending between the first housing portion 3437a and the second housing portion 3437b to enable sequential delivery of a medicament from a first medicament container assembly, which is followed by a delivery of medicament from the second medicament container assembly. In some embodiments, the passageway 3438 may be omitted and separate flow restriction members can be associated with each of the first and second medicament container assemblies to either vary or sync the delivery rates of medicament from the two medicament container assemblies.

As the elastomeric members 2217 advance distally within their respective medicament container assembly 2200, the gas vent assemblies 2310 can be transitioned from the collapsed configuration (FIGS. 37-39) to an expanded configuration (FIGS. 40-41, just prior to complete delivery of medicament), and a series of partially expanded configurations therebetween. Once the gas vent assemblies 2310 have been placed in the expanded configuration, the elastomeric members 2217 can continue to travel a final distance to deliver the remaining amount from the complete dose, which in turn pulls on the valve portions 2345. As the valve portions 2345 travel distally, the O-ring 4342 of one of the valve portions 2345 passes over and unseals the vent inlet port 3113. Once the vent inlet port 3113 is unsealed, the gas passageway 3135 is in fluid communication with the vent passageway 3114. The O-ring 4342 of the other valve portion 2345 passes over and unseals the vent outlet port 3117, thereby placing the vent passageway 3114 in fluid communication with the vent outlet port 3117 and the vent opening 3112. As the elastomeric members 2217 complete their distal travel and pulls the two valve portions, both the vent inlet port 3113 and the vent outlet port 3117 are unsealed to place the gas passageway 3135 in fluid communication with the vent opening 3112. This allows pressurized gas from the housing gas chamber of the medicament cavity 3139 to escape to the external environment such that needle retraction can occur.

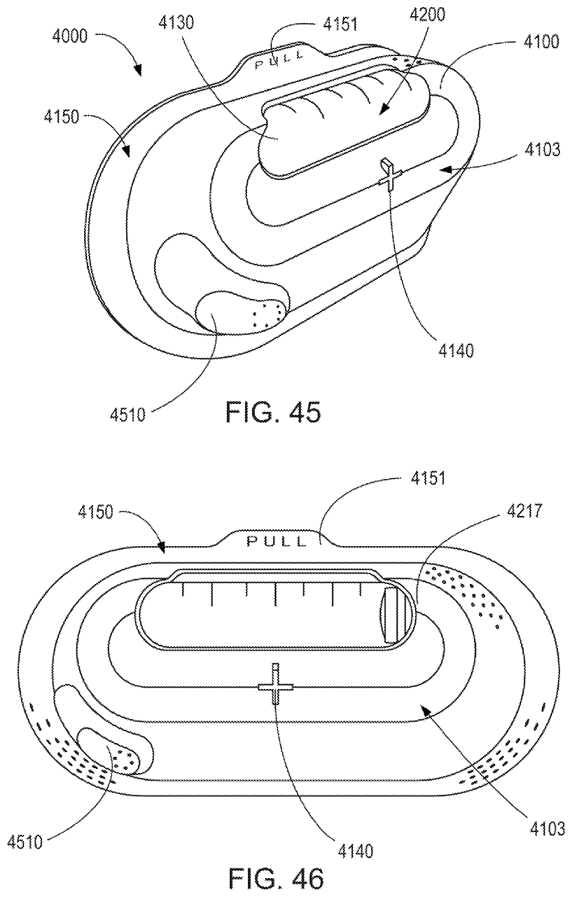

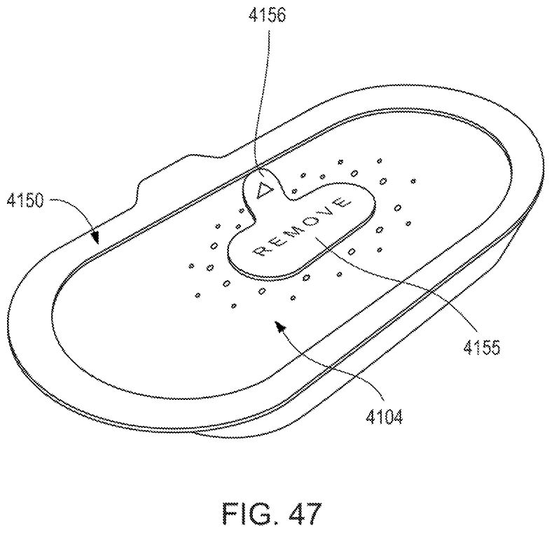

As shown in FIGS. 45-47, a medical injector 4000 includes a housing 4100 and a medicament container assembly 4200. The housing 4100 has a top portion 4103 and a bottom portion 4104. The housing includes a status indicator aperture 4130. The status indicator aperture 4130 can allow a patient to monitor the status and/or contents of the medicament container assembly 4200, a position of an elastomeric member 4217 within the medicament container assembly 4200, and/or and the medicament contained within the housing 4100. As shown in FIG. 46, the status indicator aperture 4130 allows an entire travel stroke of the elastomeric member 4217 to be viewed by the user.

The housing 4100 includes a needle alignment indicator 4140 to provide a user with visual indication as to where a needle will be deployed out of the bottom portion 4104 once the medical injector 4000 is activated. In some embodiments, the needle alignment indicator 4140 is provided with a light emitting diode (LED) to provide status information to the user. For example, the needle alignment indicator 4140 can illuminate green to indicate that the medical injector 4000 is ready for use, illuminate blue or amber to indicate that the medical injector 4000 is in use and currently dispensing medicament, and illuminate red when medicament delivery has been completed.

An adhesive portion 4150 is attached to the bottom portion 4104 and includes a peelable backing 4151 to expose an adhesive portion that can be placed on and attached to a body surface of a user. Although the adhesive portion 4150 is shown as a ring encircling the bottom portion 4104, the adhesive portion 4150 can be of any size and shape. For example, one or more adhesive portions can be provided on bottom portion 4104 and around the needle aperture (not shown). The housing 4100 includes a safety barrier 4155 secured over the needle aperture to provide a sterile barrier and prevent contamination prior to the use of the medical injector 4000. The safety barrier 4155 includes a pull tab 4156 for a user to grip and pull off the safety barrier 4155.

The medical injector 4000 includes an actuator input member 4510 configured to be depressed by a user. The actuator input member 4510 can be coupled to any system actuation assembly described herein (such as the system actuation assembly 1500, 2500, 3500) for initiating needle deployment (e.g., using the insertion member 1360, 2360, 3360 described here), and for initiating delivery of medicament from the medicament container assembly 4200 (e.g., using the needle assembly 1250, 2250, 3250).

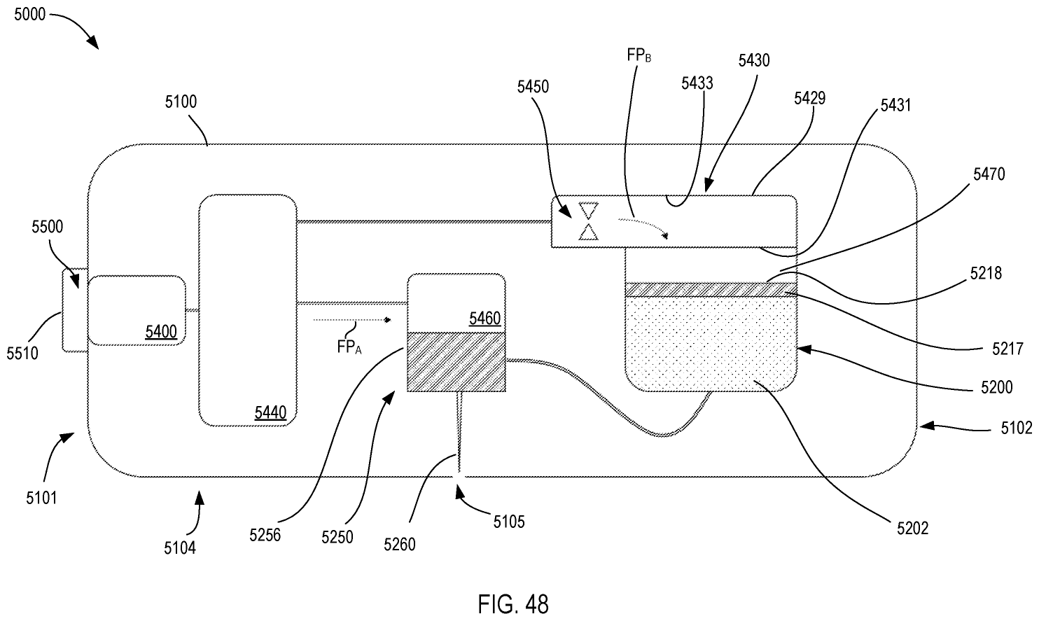

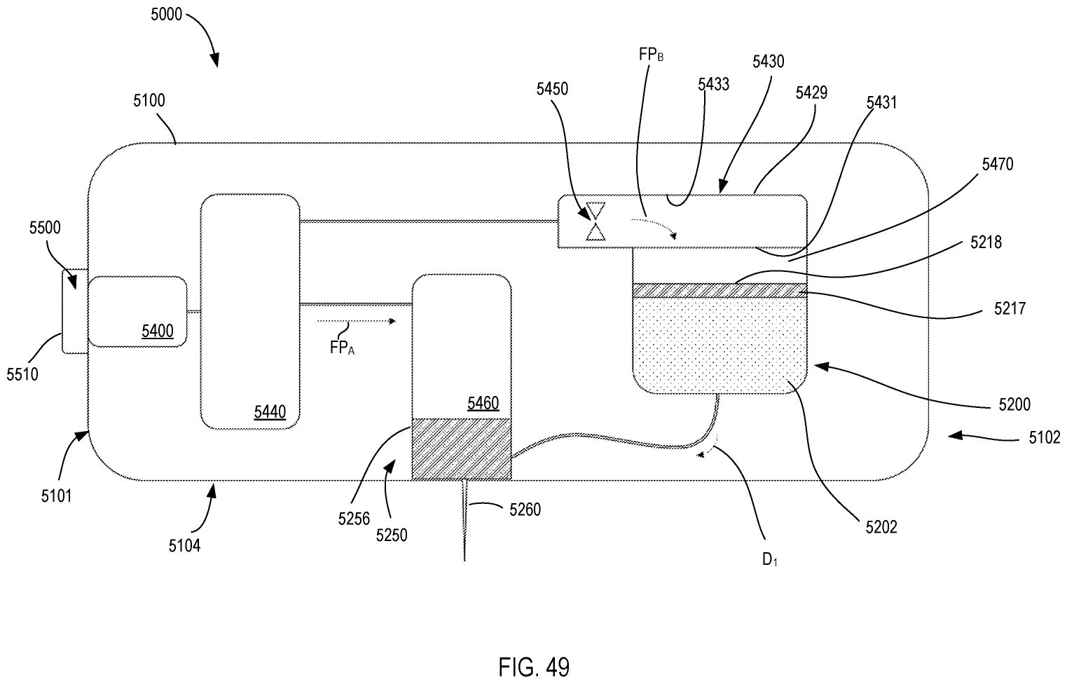

FIGS. 48 and 49 are schematic illustrations of a medicament delivery device 5000 (also referred to herein as “auto-injector,” “injector,” “medical injector,” or “device”) according to an embodiment, in a first configuration and a second configuration, respectively. The medicament delivery device 5000 includes a housing 5100, a medicament container 5200, a needle assembly 5250, an energy storage member 5400, a system actuation assembly 5500 and a flow restriction assembly 5430. As depicted in FIGS. 48 and 49, the medicament delivery device 5000 can be an auto-injector having a pistonless delivery system in which the force exerted by the gas can move the needle assembly 5250 to extend at least partially from the auto-injector and move an elastomeric member 5217 relative to (e.g., within) the medicament container 5200 to dispense a portion of a medicament 5202. Accordingly, the medicament delivery device 5000 is a gas-powered auto-injector configured to deliver a medicament 5202 contained within a medicament container 5200, as described herein. A discussion of the components of the medicament delivery device 5000 will be followed by a discussion of the operation of the medicament delivery device 5000.

The housing 5100 defines a primary gas chamber 5440 that receives a pressurned gas from the energy storage member 5400. The primary gas chamber 5440 can be of any suitable size and shape, and can, for example, be a portion of the volume defined by the housing 5100 and the flow restriction assembly 5430, The housing 5100 can be am suitable size, shape, or configuration and can be made of any suitable material. For example, in some embodiments, the housing 5100 is an assembly of multiple parts formed from a plastic material and defines a substantially rectangular shape when assembled. In other embodiments, the housing 5100 can have a substantially cylindrical shape. The housing 5100 can include a lid or end cap that can be secured to the housing via a latch or other locking mechanism so that the user can load the pre-filled syringe of pre-filled cartridge into the medical injector prior to use. In other embodiments, the medical injector is a single-use device and the lid or end cap is secured via a weld during the assembly processes. The lid can include a sealing mechanism such as an O-ring in order to ensure a tight seal onto the housing to ensure no gas escapes during activation and subsequent delivery of the medicament.

The housing 5100 includes a bottom portion 5104 extending between a first end portion 5101 and a second end portion 5102. The bottom portion 5104 includes a contact surface for contacting a body surface of a patient. The bottom portion 5104 includes a needle aperture 5105 configured to allow a needle 5260 of the needle assembly 5250 to pass through during operation. In some embodiment, the bottom portion 5104 includes an adhesive material for temporarily securing the bottom portion 5104 to the body surface of the patient. In some embodiments, the bottom portion 5104 can be covered by a removable film, which can function to protect the adhesive material and also cover the needle aperture 5105.

In some embodiments, the housing defines the primary gas chamber 5440. The primary gas chamber 5440 is configured to receive a pressurized gas from the energy storage member 5400 (e.g., a pressurized gas canister) when the energy storage member 5400 is actuated. The primary gas chamber 5440 is fluidly coupled to an insertion gas flow path (FP_{A}) defined by the housing 5100 and to a delivery gas flow path (FP_{B}). The insertion gas flow path (FP_{A}) and the delivery gas flow path (FP_{B}) are arranged in a functionally parallel arrangement. Due to the functionally parallel arrangement, a first portion of pressurized gas flows into the insertion gas flow path (FP_{A}) from the primary gas chamber 5440 concurrent with a second portion of pressurized gas that flows into the delivery gas flow path (FP_{B}) from the primary gas chamber 5440. This stands in contrast to a serial arrangement in which the pressurized gas would flow through both gas flow paths one after the other in sequence. In some embodiments, the first portion of pressurized gas has a pressure that is greater than a pressure of the second portion of the pressurized gas. In other words, in some embodiments, the pressure within the insertion gas flow path (FP_{A}) is greater than the pressure within delivery gas flow path (FP_{B}).

The insertion gas flow path (FP_{A}) is fluidly coupled to a needle actuation gas chamber 5460 that is defined, at least in part, by the needle assembly 5250. For example, the needle assembly 5250 includes a needle carrier 5256 (e.g., a needle support portion) to which the needle 5260 is coupled. The needle carrier 5256 is positioned within the housing 5100 and defines a portion of the boundary of the needle actuation gas chamber 5460. As such, the needle carrier 5256 may include a sealing member (not shown) (e.g. at least one O-ring) configured to engage the housing 5100 to form and maintain a substantially impermeable barrier therebetween.

In response to the delivery of the first portion of pressurized gas to the needle actuation gas chamber 5460 via the insertion gas flow path (FP_{A}), the needle carrier 5256 moves within the housing 5100. Specifically, the needle carrier 5256 is configured to move within the housing 5100 between a first needle carrier position as depicted in FIG. 48 and a second needle carrier position as depicted in FIG. 49. The needle 5260 is within the housing 5100 when the needle carrier 5256 is in the first needle carrier position. As such, the first needle carrier position corresponds to a first configuration of the medicament delivery device 5000. The needle carrier 5256 is in the first needle carrier position prior to actuation of the medicament delivery device 5000. The needle 5260 is outside of the housing 5100 when the needle carrier 5256 is in the second needle carrier position. As such, the second needle carrier position corresponds to a second configuration of the medicament delivery device 5000 wherein the needle 5260 extends from the bottom portion 5104 of the housing 5100. The needle carrier 5256 is in the second needle carrier position following the actuation of the medicament delivery device 5000 and during the delivery of the medicament 5202. Upon completion of the delivery of the medicament 5202 via the needle 5260, the needle carrier 5256 transitions from the second needle carrier position back towards the first needle carrier position, thereby retracting the needle 5260 within the housing 5100.

The delivery gas flow path (FP_{B}) is fluidly coupled to a medicament container gas chamber 5470 that is defined, at least in part, by the medicament container 5200 and the elastomeric member 5217. In some embodiments, the medicament container 5200 is a pre-filled syringe or pre-filled cartridge. In other embodiments, the medicament container 5200 can be a syringe or cartridge than can be filled by a user or a healthcare professional. As shown, the medicament container 5200 includes an elastomeric member 5217 positioned within the medicament container 5200 to seal the medicament 5202 within the medicament container 5200. The elastomeric member 5217 is configured to move within the medicament container 5200 in response to a pressure exerted by the second portion of the pressurized gas to convey the medicament 5202 from the medicament container 5200 to the needle assembly 5250 (as depicted by arrow Di in FIG. 49). More particularly, pressure in the medicament container gas chamber 5470 exerts a force on a first surface 5218 of the elastomeric member 5217 to move the elastomeric member 5217 within the medicament container 5200 (i.e., to expel the medicament therefrom).

The elastomeric member 5217 can be of any design or formulation suitable for contact with the medicament 5202. For example, the elastomeric member 5217 can be formulated to minimize any reduction in the efficacy of the medicament 5202 that may result from contact (either direct or indirect) between the elastomeric member 5217 and the medicament 5202. For example, in some embodiments, the elastomeric member 5217 can be formulated to minimize any leaching or out-gassing of compositions that may have an undesired effect on the medicament 5202. In other embodiments, the elastomeric member 5217 can be formulated to maintain its chemical stability, flexibility and/or sealing properties when in contact (either direct or indirect) with the medicament 5202 over a long period of time (e.g., for up to six months, one year, two years, five years or longer).

In some embodiments, the elastomeric member 5217 can be constructed from multiple different materials. For example, in some embodiments, at least a portion of the elastomeric member 5217 can be coated. Such coatings can include, for example, polydimethylsiloxane. In some embodiments, at least a portion of the elastomeric member 5217 can be coated with polydimethylsiloxane in an amount of between approximately 0.02 mg/cm² and approximately 0.80 mg/cm².

In some embodiments, the medicament delivery device includes a flow restriction assembly 5430 that defines the delivery gas flow path (FP_{B}). The flow restriction assembly 5430 includes an assembly body 5429 and a flow restriction member 5450. The assembly body 5429 has a first outer surface 5431 coupled to the medicament container 5200. The assembly body 5429 also has an inner surface 5433 that at least partially defines the delivery gas flow path (FP_{B}). As such, in some embodiments, the flow restriction member 5450 is positioned within the delivery gas flow path (FP_{B}). In other words, the flow restriction member 5450 is supported by the inner surface 5433 of the flow restriction assembly 5430.

The flow restriction member 5450 is configured to regulate a flow of the second portion of the pressurized gas into the medicament container gas chamber via the delivery gas flow path (FP_{B}). In other words, the flow restriction member 5450 is configured to regulate (e.g., limit) the flow rate of pressurized gas therethrough, which regulates (e.g., limits) the magnitude of the pressure of the second portion of the pressurized gas that acts on the elastomeric member 5217. For example, in some embodiments, the flow restriction member 5450 is calibrated with nitrogen gas (N₂) at 30 psig (inlet side) to atmosphere (outlet side) at standard temperature and pressure to have a flow rate of between 0.5 to 3 standard cubic centimeter per minute (sccm). In some embodiments, the flow restriction member 5450 is calibrated with nitrogen gas (N₂) at 30 psig (inlet side) to atmosphere (outlet side) at standard temperature and pressure to have a flow rate of between about 0.75 and 1.5 standard cubic centimeter per minute (sccm). In some embodiments, the flow restriction member 5450 is calibrated with nitrogen gas (N₂) at 30 psig (inlet side) to atmosphere (outlet side) at standard temperature and pressure to have a flow rate of about 1 standard cubic centimeter per minute (sccm). As described herein, standard temperature is 60° F. (15.6° C.) and standard pressure is 14.696 psia (101.3 kPa).

In some embodiments, the compressed gas supplied by the energy storage member 5400 is an argon gas and the flow restriction member 5450 has a flow rate rating of about 0.75 and 1.5 sccm based on the nitrogen gas calibration described above. In some embodiments, the compressed gas supplied by the energy storage member 5400 is an argon gas and the flow restriction member 5450 has a flow rate rating of about 1 sccm based on the nitrogen gas calibration described above. In some embodiments, the compressed gas in the energy storage member 5400 has a molecular weight greater than the molecular weight of argon. For example, in some embodiments, the compressed gas supplied by the energy storage member 5400 is R134a (Tetrafluoroethane) and the flow restriction member 5450 has a flow rate rating of about 10 to 100 sccm based on the nitrogen gas calibration described above. In some embodiments, the compressed gas is R134a (Tetrafluoroethane) and the flow restriction member 5450 has a flow rate rating of about 20 to 40 sccm based on the nitrogen gas calibration described above.

In some embodiments, the flow rate of the medicament 5202 can be reduced to less than 0.2 mL/sec (or in some embodiments between 0.05 mL/sec and 0.01 mL/see) using gas pressure that is supplied to medicament container gas chamber 5470 via the delivery gas flow path (FP_{B}) and the flow restriction member 5450. The lower injection forces and/or slower delivery (compared with pressures supplied directly from the primary gas chamber 5440) can produce laminar flow of the medicament 5202 through the needle, prevent shearing of high molecular weight compounds in the medicament 5202, and/or reduce pain sensed by a patient particularly if the medicament 5202 being delivered is very high viscosity (e.g., greater than about 100 centipoise at room temperature). Additionally, the lower injection forces may facilitate the delivery of relatively large volumes of the medicament 5202, such as dosages of greater than 30 mL (e.g., 40 mL).

In some embodiments, the medicament delivery device 5000 includes the system actuation assembly 5500. The system actuation assembly 5500 includes an input surface 5510 configured to receive a force input. The input force is then transmitted to an inclined plane (not shown), a release member (not shown), or other similar structure configured to initiate a release of the pressurized gas from the energy storage member 5400. For example, in some embodiments, the inclined plane is coupled to the input surface 5510 and is positioned to develop a linear motion of the energy storage member 5400 in response to the force input. The linear motion results in the activation (e.g., puncturing) of the energy storage member 5400. In this manner, a single motion can actuate the energy storage member 5400, which, in turn, supplies pressurized gas via two different flow paths to perform the different functions of inserting the needle and producing a controlled delivery of the medicament.

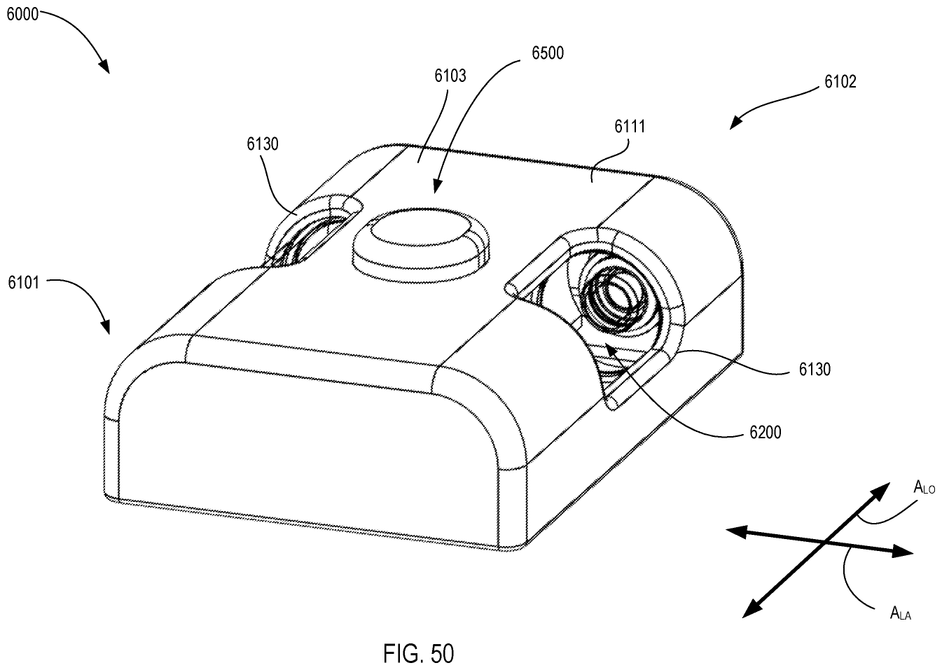

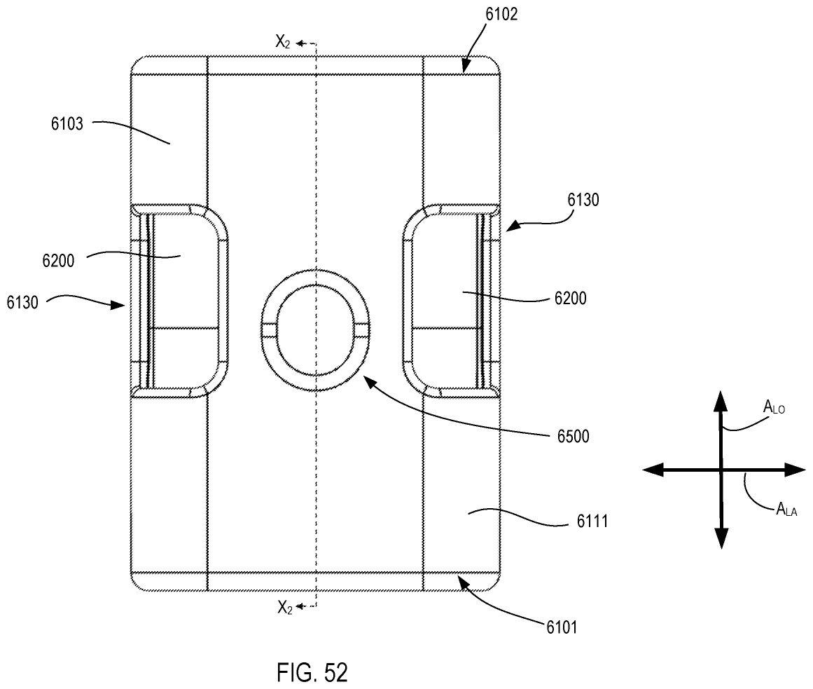

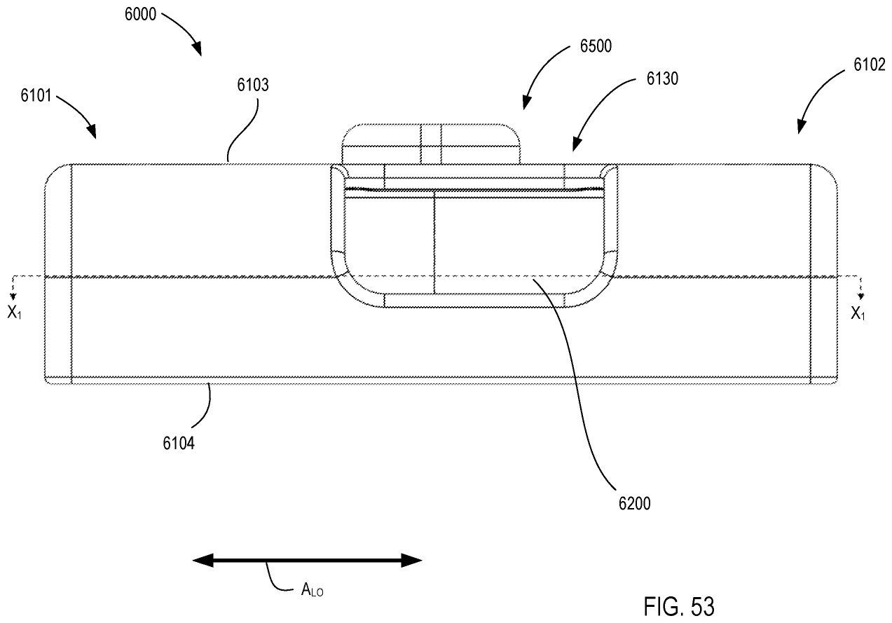

In some embodiments, a medicament delivery device can be an auto-injector having a pistonless delivery system in which the force exerted by the gas can move a needle assembly to extend at least partially from the auto-injector and move an elastomeric member relative to (e.g., within) the medicament container assembly. For example, FIGS. 50-68 show a medicament delivery device 6000 (also referred to herein as “auto-injector,” “injector,” “medical injector,” or “device”), according to an embodiment. Although configuration of the medicament delivery device 6000 is shown as being an on-body delivery device, in other embodiments any of the components (and their functionality) can be included in an auto-injector of the types shown and described in International Patent Publication No. 2021/030210, which is incorporated herein by reference in its entirety. The medicament delivery device 6000 is a gas-powered device configured to deliver a medicament contained within at least one medicament container 6200 (e.g., a medicament container assembly), as described herein. In some embodiments, the medicament container 6200 is a pre-filled syringe or pre-filled cartridge. In other embodiments, the medicament container 6200 can be a syringe or cartridge than can be filled by a user or a healthcare professional.

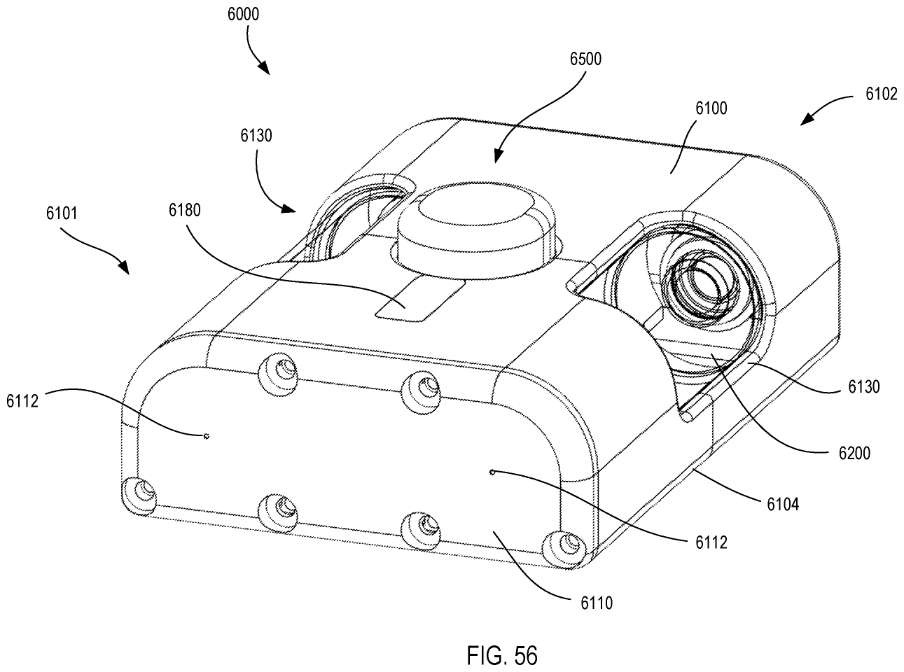

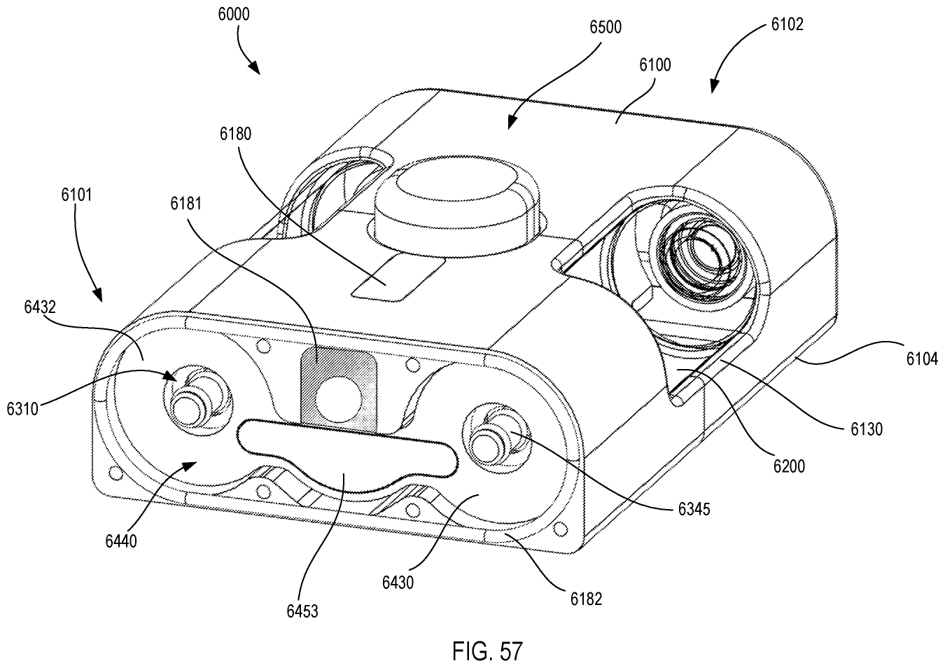

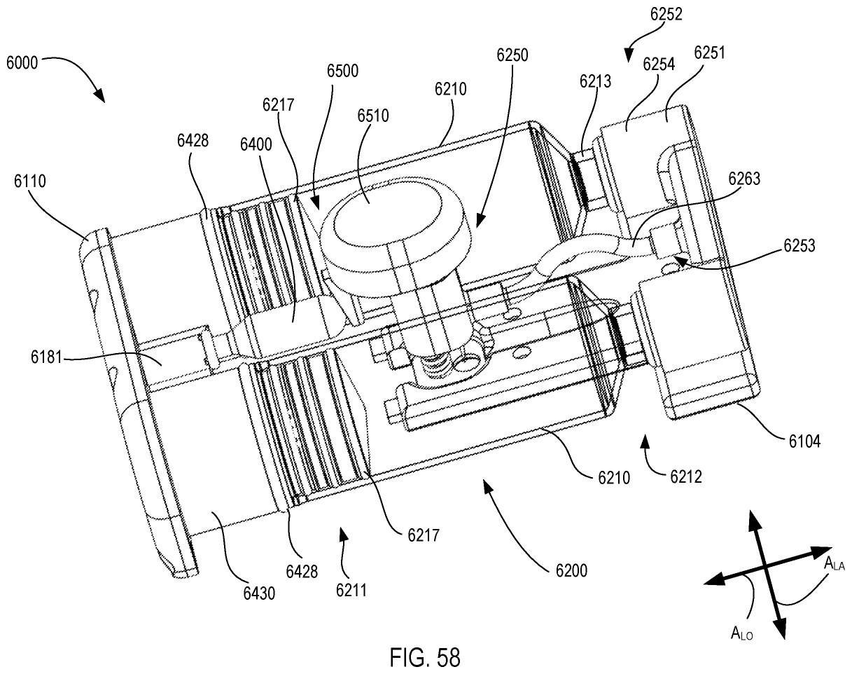

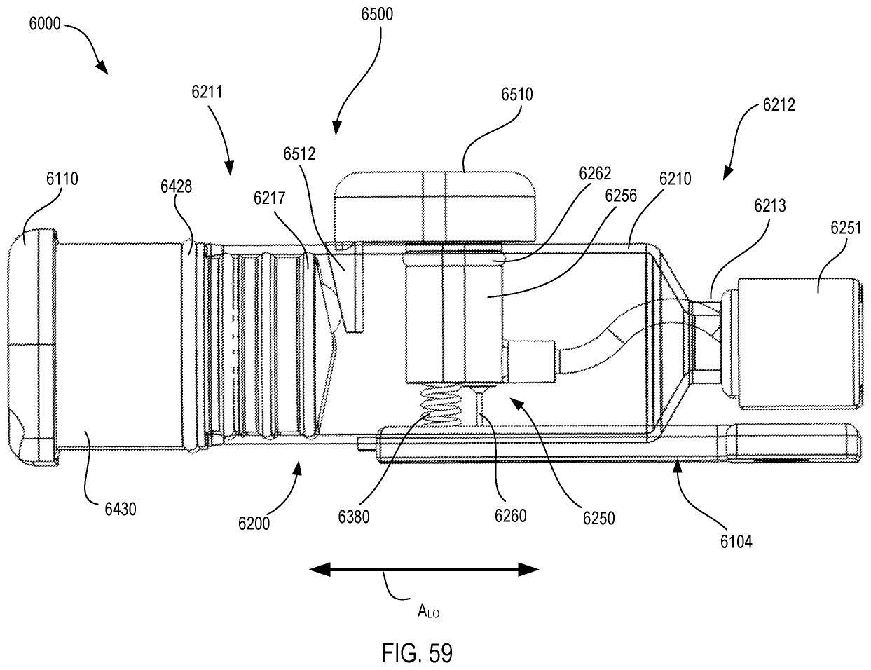

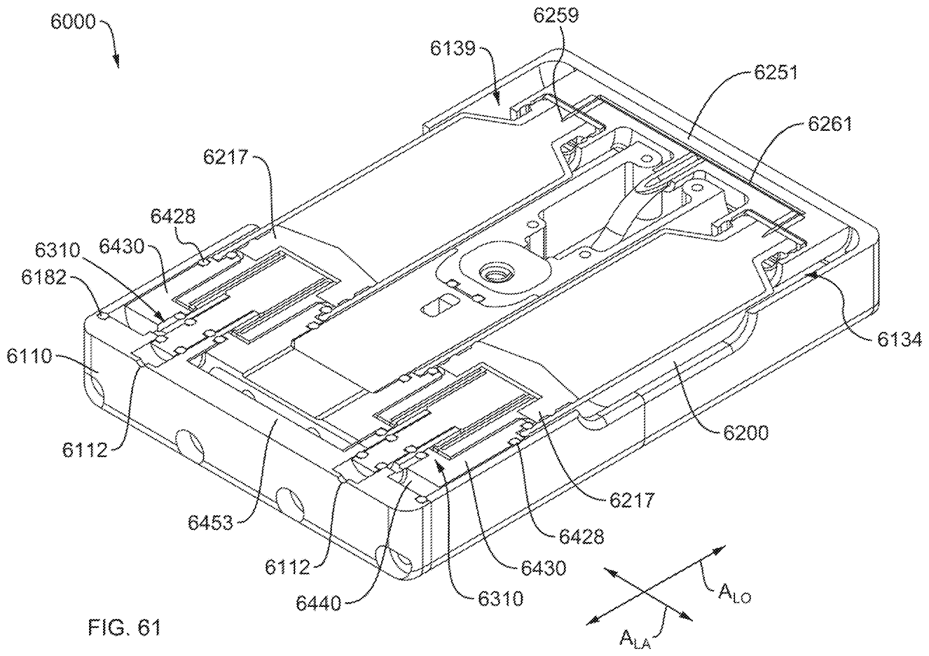

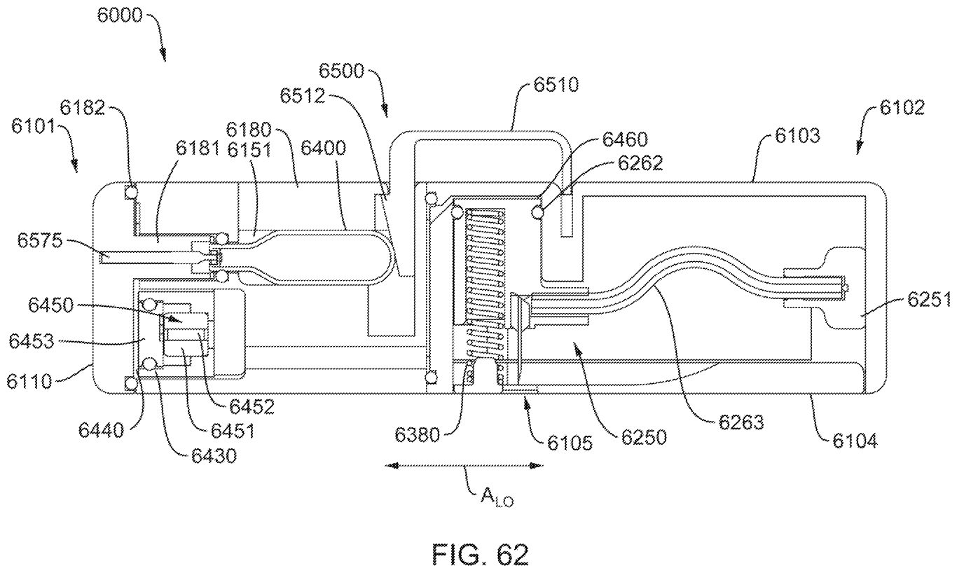

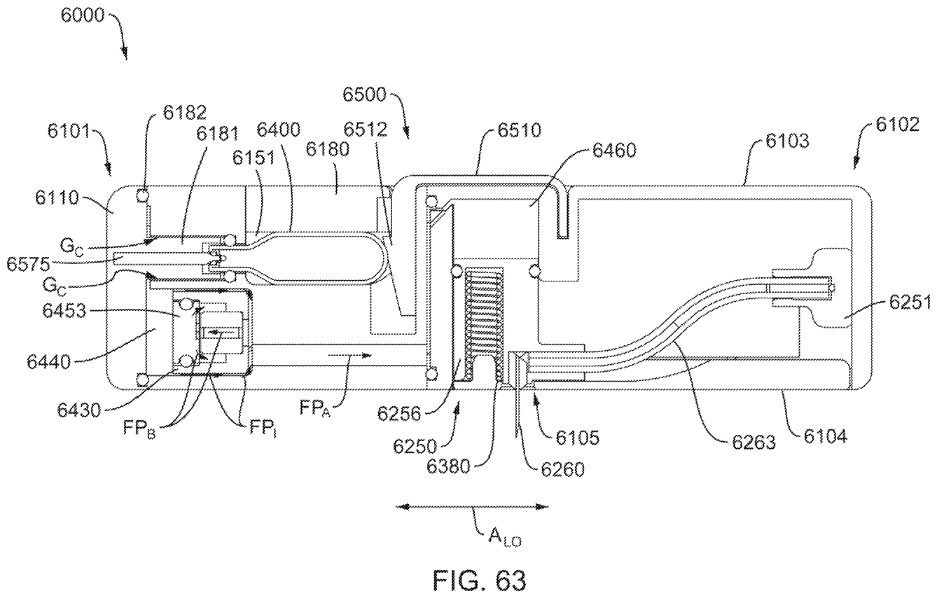

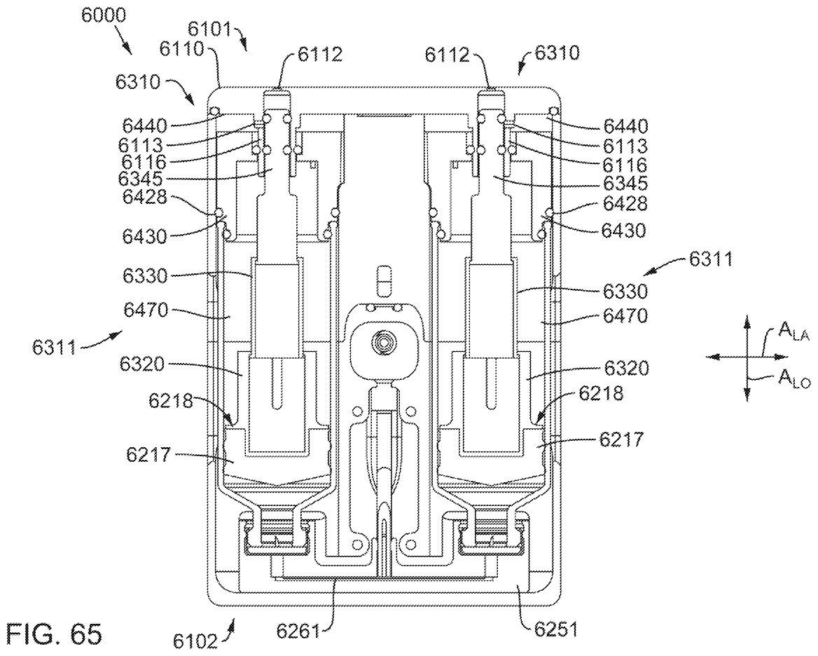

FIGS. 50-68 show a medicament delivery device 6000 (also referred to as a medical injector or on body delivery device) according to an embodiment. The medicament delivery device 6000 includes a housing 6100 (see e.g., FIG. 56), that is secured within an outer cover 6111. The housing 6100 may be maintained within the outer cover 6111 during the delivery of the medicament. The medicament delivery device 6000 also includes a system actuation assembly 6500 (see e.g., FIGS. 58, 59, 62, and 63), an energy storage member 6400 (see e.g., FIGS. 62, and 63), a medicament container 6200 (see e.g., FIGS. 58 and 59), a needle assembly 6250 (see e.g., FIGS. 59, 62, and 63), and a flow restriction assembly 6430 (see e.g., FIGS. 58, 59, and 64). In some embodiments, the medicament delivery device 6000 also includes a medicament coupling member 6251 (see e.g., FIG. 61) and/or at least one vent assembly 6310 (see e.g., FIGS. 65-68). The medicament coupling member 6251 defines, at least in part, a flow passageway 6261 between the medicament container 6200 and the needle assembly 6250. The vent assembly 6310 is configured to place a primary gas chamber 6440 (see e.g., FIGS. 57 and 61-63) defined by the housing 6100 in fluid communication with an exterior volume surrounding the housing 6100 so as to release the pressurized gas from the primary gas chamber 6440 to the exterior volume.

As shown in FIGS. 50-56, the medicament delivery device 6000 has a first end portion 6101 and a second end portion 6102. The first end portion 6101 and the second end portion are separated by a distance along a longitudinal axis (A_{LO}) and have a width along a lateral axis (A_{LA}). The medicament delivery device 6000 includes a top portion 6103 extending between the first end portion 6101 and a second end portion 6102 along the longitudinal axis (A_{LO}). The top portion 6103 defines at least one status indicator aperture 6130. The status indicator aperture 6130 can allow a patient to monitor the status and/or contents of the medicament container 6200, a position of an elastomeric member 6217 within the medicament container 6200, and/or the medicament contained within the medicament delivery device 6000. For example, by visually inspecting the status indicator apertures 6130, a patient can determine whether the medicament container 6200 contains a medicament and/or whether the medicament has been dispensed. In some embodiments, the status indicator aperture 6130 includes a length along the longitudinal axis (A_{LO}) to display an entire, or substantially the entire, stroke length of the elastomeric member 6217 during operation.

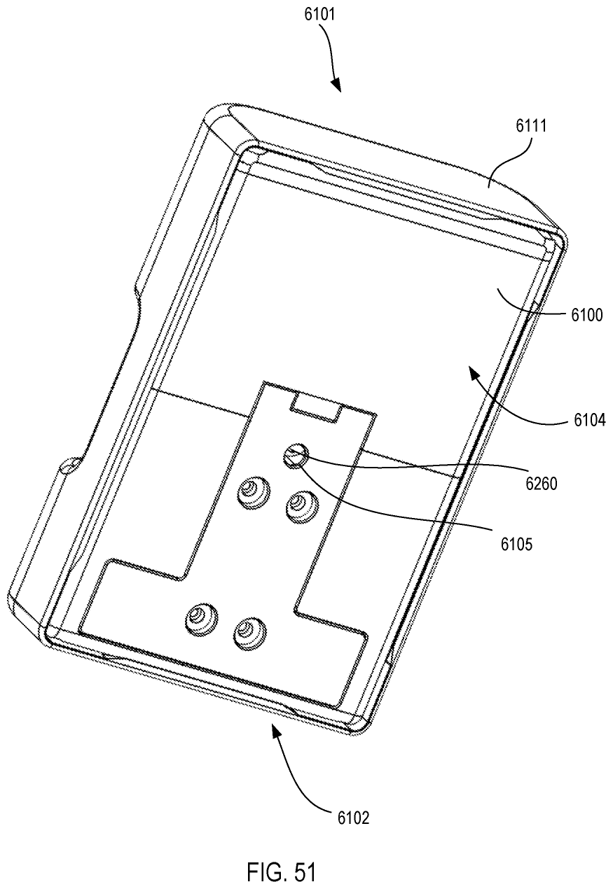

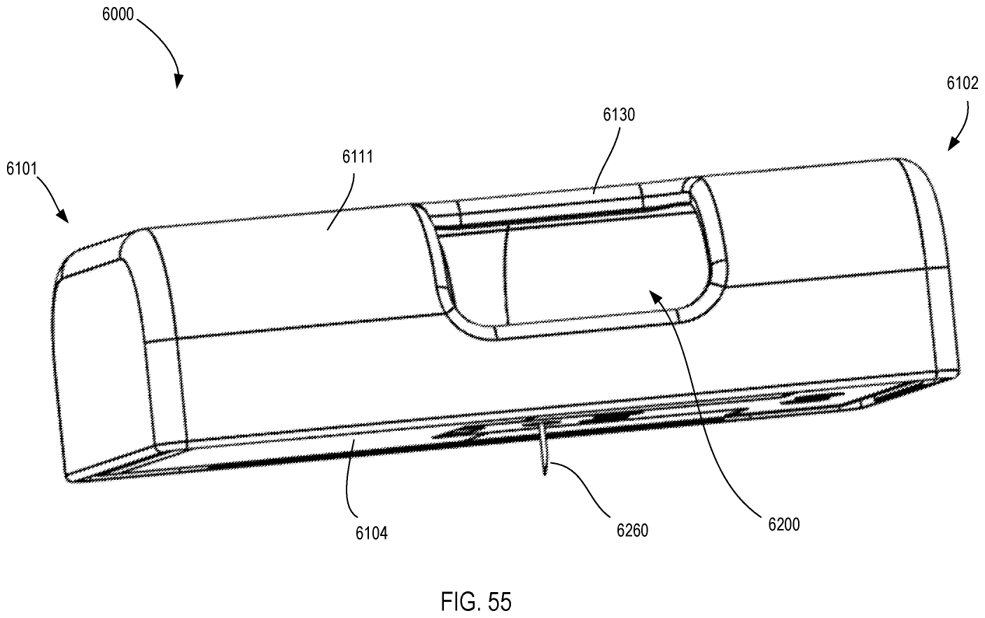

As particularly illustrated in FIGS. 51 and 55, the housing 6100 includes a bottom portion 6104 extending between the first end portion 6101 and a second end portion 6102. The bottom portion 6104 includes a contact surface for contacting a body surface of a patient. The bottom portion 6104 includes a needle aperture 6105 configured to allow a needle 6260 of the needle assembly 6250 to pass through during operation. In some embodiments, the bottom portion 6104 includes an adhesive material for temporarily securing the bottom portion 6104 to the body surface of the patient. In some embodiments, the bottom portion 6104 can be covered by a removable film, which can function to protect the adhesive material and also to cover the needle aperture 6105.

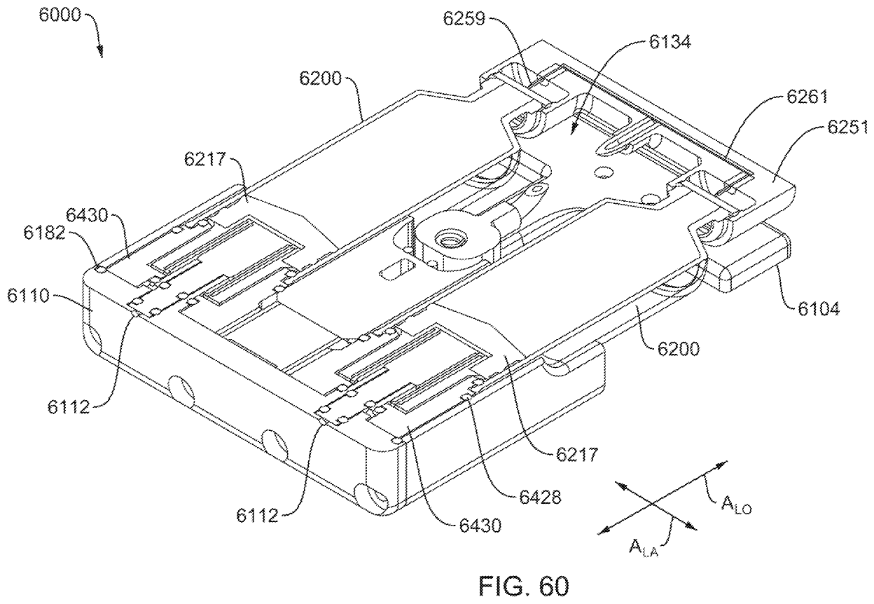

As shown in FIGS. 60 and 61, the housing 6100 defines at least one medicament cavity 6139. In the depicted embodiment, the housing 6100 defines two medicament cavities, each of which contains a medicament container 6200. Only one of the medicament containers is labeled with reference characters and described below, but is should be understood that each container is similar in structure and functionality. In other embodiments, any of the devices described herein can include any suitable number of medicament containers (e.g., one, two, three, four, or more) with the operation of the device being similar that which is described herein. The medicament cavity 6139 is configured to receive the medicament container 6200 and at least a portion of the medicament coupling member 6251. The medicament container 6200 is configured to move within the medicament cavity 6139 from a first container position, as depicted in FIG. 60, to a second container position, as depicted in FIG. 61. For example, the medicament container 6200 may be configured to move linearly along the longitudinal axis (A_{LO}). The housing 6100 can include a lid or cover that can be secured to the housing via a latch or other locking mechanism so that the user can load the pre-filled syringe of pre-filled cartridge into the medical injector prior to use. The lid can include a sealing mechanism, such as an O-ring, to ensure a tight seal onto the housing and to ensure no gas escapes during employment of the medicament delivery device 6000.

Each medicament container 6200 contains a medicament and includes an elastomeric member 6217 (see e.g., FIG. 58) that seals the medicament within the medicament container 6200. The medicament container and the elastomeric member 6217 define a medicament container gas chamber 6470 (see e.g., FIG. 65).

The needle assembly 6250 is also disposed within the housing 6100. The needle assembly 6250 includes a needle carrier 6256 with a needle 6260 coupled thereto. (See e.g., FIGS. 59 and 63). The needle carrier 6256 defines a portion of a boundary of a needle actuation gas chamber 6460 (see e.g., FIGS. 62 and 63). The needle carrier 6256 is configured to move within the housing 6100 between a first needle carrier position, such as depicted in FIG. 62, and a second needle carrier position, such as depicted in FIG. 63. The needle 6260 is within the housing 6100 when the needle carrier 6256 is in the first needle carrier position and is outside the housing 6100 when the needle carrier 6256 is in the second needle carrier position.

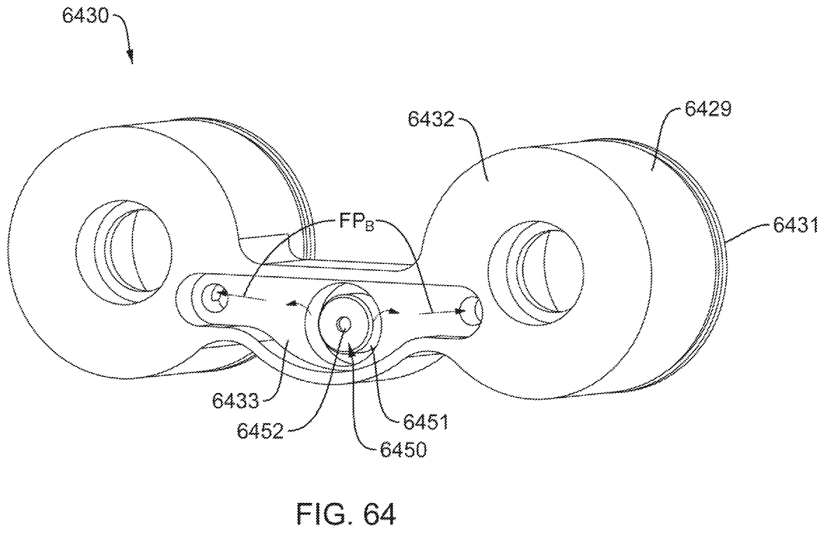

Additionally, the flow restriction assembly 6430 is disposed within the housing 6100. The flow restriction assembly 6430 defines a portion of a boundary of the primary gas chamber 6440 (see e.g., FIGS. 57 and 63) and a delivery gas flow path (FP_{B}) (see e.g., FIGS. 63 and 64). The flow restriction assembly 6430 is configured to move within the housing 6100 to move the medicament container 6200 from a first container position, such as depicted in FIG. 60, to a second container position, such as depicted in FIG. 61. The needle carrier 6256 is in fluid communication with the medicament container 6200 when the medicament container 6200 is in the second container position. Further, the energy storage member 6400 is positioned within the housing 6100 and configured to produce a pressurized gas when the energy storage member 6400 is actuated. A first portion of the pressurized gas flows within the insertion gas flow path (FP_{A}) and into the needle actuation gas chamber 6460 to move the needle carrier 6256 from the first needle carrier position to the second needle carrier position. A second portion of the pressurized gas flows through the delivery gas flow path (FP_{B}) and into the medicament container gas chamber 6470 to move the elastomeric member 6217 within the medicament container 6200.

As shown in FIGS. 62 and 63, the housing 6100 defines an energy storage member cavity 6151. The energy storage member cavity 6151 is configured to receive the energy storage member 6400 (e.g., a gas container) and a portion of the system actuation assembly 6500 (e.g., an inclined plane 6512). A retention member 6180 is configured to be inserted into the energy storage member cavity 6151 to retain the energy storage member 6400 within the cavity. In some embodiments, the energy storage member cavity 6151 is fluidically isolated from the primary gas chamber 6440 by a sealing member (e.g., an O-ring) that circumscribes the energy storage member 6400. When the pressurized gas is released from the energy storage member, the sealing member prevents the pressurized gas from entering the energy storage member cavity 6151 as the energy storage member cavity 6151 may be in fluid communication with the exterior volume surrounding the housing 6100.

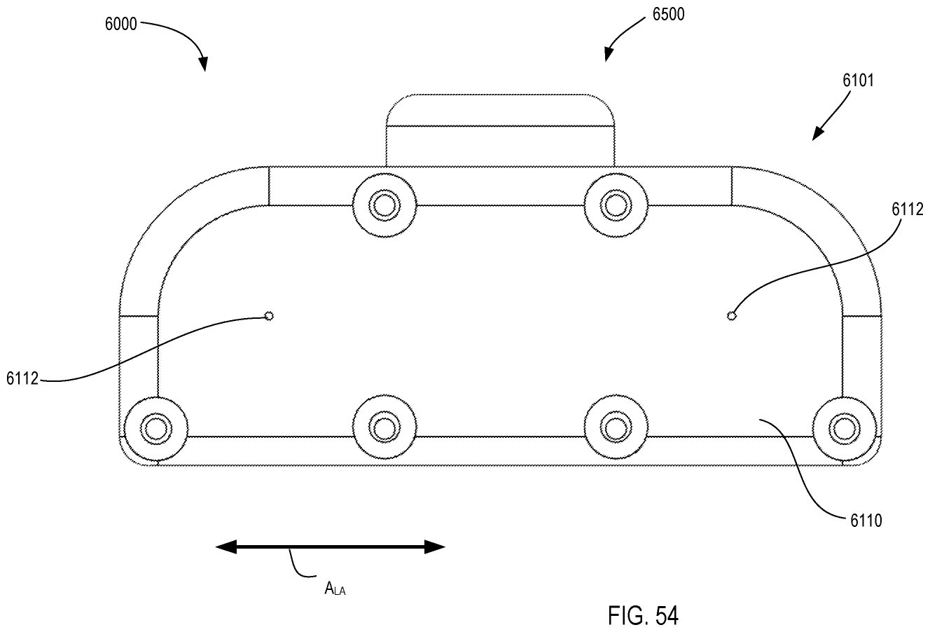

The housing 6100 defines the primary gas chamber 6440 and an insertion gas flow path (FP_{A}) (see e.g., FIG. 63). The first end portion 6101 of the housing 6100 includes a housing cap 6110 (see e.g., FIGS. 54 and 56). The housing cap 6110 defines a boundary of the primary gas chamber 6440. Accordingly, in some embodiments, the housing cap 6110 includes an O-ring 6182 (see e.g., FIG. 57 wherein the housing cap 6110 is removed for clarity) to fluidically seal the housing cap 6110 to adjacent portions of the housing 6100. As shown in FIG. 60, the housing cap 6110 defines an outlet orifice 6112 of the vent assembly 6310. The outlet orifice 6112 provides a passageway through which pressurized gas is conveyed from the primary gas chamber 6440 to a volume outside of the medicament delivery device 6000. In this manner, the force produced by the pressurized gas on the needle assembly 6250 can be reduced to allow needle retraction after the injection is completed. Although the outlet orifice 6112 is shown as being defined by the housing cap 6110, in other embodiments, the outlet orifice 6112 (and any of the vent openings described herein) can be defined within any suitable portion of the housing 6100. For example, in some embodiments, the outlet orifice 6112 (and any of the vent openings described herein) can be defined by the housing cap 6110 but can have a centerline that is nonparallel to a longitudinal axis (A_{LO}) of the medicament delivery device 6000. Said another way, in some embodiments, the outlet orifice 6112 (and any of the vent openings described herein) can open towards a side of the medical injector, rather than opening towards the first end portion 6101, as shown. In other embodiments, the outlet orifice 6112 (and any of the vent openings described herein) can be defined by any wall and/or surface of the housing 6100. In some embodiments, the outlet orifice 6112 is provided on the bottom portion 6104.

The bottom portion 6104 of the housing 6100 includes a contact surface with a needle aperture 6105. The contact surface is configured to contact a body surface of a patient and stabilize the medicament delivery device 6000 against the body surface during operation. In some embodiments, the contact surface may be provided with an adhesive patch or material to further secure the bottom portion 6104 of the housing 6100 to the body surface of a patient. In some embodiments, a cover or a guard (not shown) may be provided over the needle aperture 6105 to prevent ingress of foreign matter into the housing 6100 through the needle aperture 6105, to maintain needle sterility, and/or to prevent accidental needle prick. In some embodiments, the adhesive patch or material may include a protective film or backing that may be removed by the patient prior to securing the bottom portion 6104 onto the body surface of the patient. In some embodiments, the protective film is coupled to the needle aperture's cover or guard such that removal of the protective film from the adhesive patch also removes the cover or guard from the needle aperture. In some embodiments, a portion of the protective film is attached securely to the system actuation assembly 6500 to prevent actuation until the protective film is removed from the bottom portion 6104 and/or removed from the system actuation assembly 6500. In some embodiments, the bottom portion 6104 may be coupled to a protective garment worn by the patient.

FIGS. 62-64 illustrate portions of the gas-powered system employed by the medicament delivery device 6000 to deliver the medicament. Specifically, FIG. 62 depicts a cross-sectional view of the medicament delivery device 6000 in a first configuration prior to actuation, the medicament delivery device 6000 of FIG. 63 is in a second configuration following actuation, while FIG. 64 is a perspective view of the flow restriction assembly 6430. As illustrated, in some embodiments, the housing 6100 defines the primary gas chamber 6440. The primary gas chamber 6440 is configured to receive a pressurized gas from the energy storage member 6400 (e.g., a pressurized gas canister) when the energy storage member 6400 is actuated. For example, as depicted in FIG. 63, when actuated, the energy storage member 6400 is breached by a penetrator 6575 that is supported by the housing cap 6110. Once breached, the pressurized gas flows from the energy storage member 6400 through a clearance (G_{C}) between a protrusion 6181 of the housing cap 6110 and an adjacent portion of the housing 6100. A sealing member circumscribing the energy storage member 6400 prevents the pressurized gas from exiting the energy storage member 6400 and entering the energy storage member cavity 6151. Accordingly, the pressurized gas exits the energy storage member 6400 and enters the primary gas chamber 6440 via the clearance (G_{C}).

As depicted, in some embodiments, the primary gas chamber 6440 is defined within the housing 6100. In order to define the primary gas chamber 6440, the housing cap 6110 is fluidically sealed to adjacent portions of the housing 6100 by the O-ring 6182. Additionally, the flow restriction assembly 6430 is positioned between a portion of the housing cap 6110 and the second end portion 6102, such as depicted in FIG. 57 in which a majority of the housing cap 6110 has been removed for clarity. An outer surface (e.g., the second outer surface 6432) of an assembly body 6429 of the flow restriction assembly 6430 forms a portion of the boundary of the primary gas chamber 6440. At least a portion of the assembly body 6429 is circumscribed by a sealing member 6428 that establishes a slidable seal with the housing 6100.

As depicted in FIG. 62, the primary gas chamber 6440 has a first longitudinal dimension prior to the actuation of the medicament delivery device 6000. As depicted in FIG. 63, the primary gas chamber 6440 has a second longitudinal dimension following the actuation of the medicament delivery device 6000. The second longitudinal dimension has a greater magnitude than the first longitudinal dimension. The change in magnitude corresponds to a longitudinal movement of the flow restriction assembly 6430 in response to the gas pressure within the primary gas chamber 6440, while the housing cap 6110 remains at a fixed longitudinal position. Said another way, the introduction of the pressurized gas into the primary gas chamber 6440 results in an expansion of the volume of the primary gas chamber 6440.

As depicted in FIG. 63, the primary gas chamber 6440 is fluidly coupled to the insertion gas flow path (FP_{A}) defined by the housing 6100 and to the delivery gas flow path (FP_{B}) defined by the flow restriction assembly 6430. The insertion gas flow path (FP_{A}) and the delivery gas flow path (FP_{B}) are arranged in a functionally parallel arrangement. Due to the functionally parallel arrangement, a first portion of pressurized gas flows into the insertion gas flow path (FP_{A}) from the primary gas chamber 6440 concurrent with a second portion of pressurized gas that flows into the delivery gas flow path (FP_{B}) from the primary gas chamber 6440. This stands in contrast to a serial arrangement in which the pressurized gas would flow through both gas flow paths one after the other. In some embodiments, the first portion of pressurized gas has a pressure that is greater than a pressure of the second portion of the pressurized gas. In other words, in some embodiments, the pressure within the insertion gas flow path (FP_{A}) is greater than the pressure within delivery gas flow path (FP_{B}). The pressure within the insertion gas flow path (FP_{A}) may be optimized based on the force required to insert the needle 6260. Similarly, the pressure within delivery gas flow path (FP_{B}) may be optimized to deliver the medicament at a desired rate.

In some embodiments, the utilization of the functionally parallel arrangement of the insertion gas flow path (FP_{A}) and the delivery gas flow path (FP_{B}) facilitates the rapid insertion of the needle 6260 upon actuation of the medicament delivery device 6000, while the pressure in the medicament container gas chamber 6470 is more slowly increased. This ensures that the needle 6260 is inserted into the patient prior to the delivery of the medicament. Said another way, the functionally parallel arrangement of the insertion gas flow path (FP_{A}) and the delivery gas flow path (FP_{B}) effectively decouples operations driven by the first portion of the pressurized gas (e.g., needle insertion) from those driven by the second portion of the pressurized gas (e.g., movement of the elastomeric member 6217). This arrangement allows for a single energy storage member to provide forces (e.g., pressurized gas) to accomplish multiple separate functions associated with medicament delivery.

As depicted in FIG. 63, the primary gas chamber 6440 is fluidly coupled to the insertion gas flow path (FP_{A}) and the delivery gas flow path (FP_{B}) via an intermediate gas flow path (FP_{I}). The intermediate gas flow path (FP_{I}) is disposed downstream of the primary gas chamber 6440. The intermediate gas flow path (FP_{I}) is disposed upstream of the insertion gas flow path (FP_{A}) and the delivery gas flow path (FP_{B}). In other words, the intermediate gas flow path (FP_{I}) is disposed between the primary gas chamber 6440 and the insertion and delivery gas flow paths (FP_{A}. FP_{B}). The intermediate gas flow path (FP) defined by a clearance between a portion of the flow restriction assembly 6430 and the housing 6100. For example, a central portion of the flow restriction assembly 6430 may lack a sealing member and may have an outer dimension that establishes a clearance with adjacent portion of the housing. The resulting clearance may permit the passage of the pressurized gas.

The insertion gas flow path (FP_{A}) is fluidly coupled to a needle actuation gas chamber 6460 that is defined, at least in part, by the needle assembly 6250. For example, the needle assembly 6250 includes a needle carrier 6256 (e.g., a needle support portion) to which the needle 6260 is coupled. The needle carrier 6256 is positioned within the housing 6100 and defines a portion of the boundary of the needle actuation gas chamber 6460. As such, the needle carrier 6256 may include an actuation chamber sealing member 6262 (e.g. at least one O-ring) configured to engage the housing 6100 to form and maintain a substantially impermeable barrier therewith.

In response to the delivery of the first portion of pressurized gas to the needle actuation gas chamber 6460 via the insertion gas flow path (FP_{A}), the pressure in the needle actuation gas chamber 6460 increases. When the pressure in the needle actuation gas chamber 6460 achieves to a magnitude that exceeds a force exerted on the needle carrier 6256 by a retraction spring 6380, the needle carrier 6256, which is at least partially surrounded by the housing 6100, moves within the housing 6100. Specifically, the needle carrier 6256 is configured to move within the housing 6100 between a first needle carrier position as depicted in FIG. 62 and a second needle carrier position as depicted in FIG. 63. The needle 6260 is within the housing 6100 when the needle carrier 6256 is in the first needle carrier position. As such, the first needle carrier position corresponds to a first configuration of the medicament delivery device 6000. The needle carrier 6256 is in the first needle carrier position prior to actuation of the medicament delivery device 6000. The needle 6260 is outside of the housing 6100 when the needle carrier 6256 is in the second needle carrier position. As such, the second needle carrier position corresponds to a second configuration of the medicament delivery device 6000 wherein the needle 6260 extends from the bottom portion 6104 of the housing 6100. The needle carrier 6256 is in the second needle carrier position following the actuation of the medicament delivery device 6000 and during the delivery of the medicament. Upon completion of the delivery of the medicament via the needle 6260, the needle carrier 6256 transitions from the second needle carrier position back towards the first needle carrier position, thereby retracting the needle 6260 within the housing 6100.

In some embodiments, the movement of the needle carrier 6256 is along an axis that is orthogonal to a plane defined by the longitudinal axis (A_{LO}) and the lateral axis (A_{LA}). This movement of the needle carrier 6256 is driven by the introduction of the first portion of pressurized gas to the needle actuation gas chamber 6460 via the insertion gas flow path (FP_{A}). For example, as depicted in FIG. 62, the needle actuation gas chamber 6460 has a first volume in the first needle carrier position (e.g., prior to the delivery of the first portion of the pressurized gas or following the venting of the pressurized gas). As depicted in FIG. 63, the needle actuation gas chamber has a second volume in the second needle carrier position. The second volume is greater than the first volume.

In some embodiments, the magnitude of the pressure of the first portion of pressurized gas may be regulated (or controlled) based on an anticipated resistance to needle insertion. In other words, the magnitude may be sufficient to generate a force on the needle 6260 that is greater than a resistive force of a surface through which the needle 6260 is inserted. For example, the pressure of the first portion of the pressurized gas may develop a force on the needle 6260 that facilitates the passage of the needle through a garment, such as a protective ensemble, worn by the patient.

The delivery gas flow path (FP_{B}) is fluidly coupled to one (or more) medicament container gas chambers 6470 that are each defined, at least in part, by the medicament container 6200 and the elastomeric member 6217. For example, the medicament container 6200 includes an elastomeric member 6217 positioned within the medicament container 6200 to seal the medicament within the medicament container 6200. The elastomeric member 6217 is configured to move within the medicament container 6200 in response to a pressure exerted by the second portion of the pressurized gas to convey the medicament from the medicament container 6200. More particularly, pressure in the medicament container gas chamber 6470 exerts a force on a first surface 6218 (see e.g., FIG. 65) of the elastomeric member 6217 to move the elastomeric member 6217 within the medicament container 6200 (i.e., to expel the medicament therefrom).

The elastomeric member 6217 can be of any design or formulation suitable for contact with the medicament. For example, the elastomeric member 6217 can be formulated to minimize any reduction in the efficacy of the medicament that may result from contact (either direct or indirect) between the elastomeric member 6217 and the medicament. For example, in some embodiments, the elastomeric member 6217 can be formulated to minimize any leaching or out-gassing of compositions that may have an undesired effect on the medicament. In other embodiments, the elastomeric member 6217 can be formulated to maintain its chemical stability, flexibility and/or sealing properties when in contact (either direct or indirect) with the medicament over a long period of time (e.g., for up to six months, one year, two years, five years or longer).

In some embodiments, the elastomeric member 6217 can be constructed from multiple different materials. For example, in some embodiments, at least a portion of the elastomeric member 6217 can be coated. Such coatings can include, for example, polydimethylsiloxane. In some embodiments, at least a portion of the elastomeric member 6217 can be coated with polydimethylsiloxane in an amount of between approximately 0.02 mg/cm² and approximately 0.80 mg/cm².

In FIGS. 58 and 59, portions of the housing 6100 have been removed to illustrate the internal components of the medicament delivery device 6000, while FIGS. 60 and 61 are cross-sectional views of the medicament delivery device 6000 taken along a plane defined by the longitudinal axis (A_{LO}) and the lateral axis (A_{LA}). As depicted, in some embodiments, the medicament container 6200 includes a container body 6210. The medicament container 6200 has a first end portion 6211 and a second end portion 6212. The medicament container 6200 defines a volume that contains (i.e., is filled with or partially filled with) a medicament. The second end portion 6212 of the medicament container 6200 includes a neck 6213 that is movably coupled to the medicament coupling member 6251. The first end portion 6211 of the medicament container 6200 includes the elastomeric member 6217 that seals the medicament within the container body 6210. The elastomeric member 6217 is configured to move within the container body 6210 to convey the medicament from the second end portion 6212.

In some embodiments, the first end portion 6211 of the medicament container 6200 may include a flange (not shown) configured to be disposed within a portion of the medicament cavity 6139. The flange can be of any suitable size and/or shape. The flange may fully or partially circumscribe the container body 6210. In yet other embodiments, the medicament container 6200 need not include any flange (see, e.g., the container body 6210 described herein).

The medicament container 6200 can have any suitable size (e.g., length and/or diameter) and can contain any suitable volume of the medicament. In some embodiments, the medicament container 6200 (and any of the medicament containers and medicament container assemblies described herein) can be a cartridge having a sealed end portion. The container body 6210 can be constructed from any suitable materials including but is not limited to, glass, cyclic olefin copolymer (COC), and cyclic olefin polymer (COP).

In some embodiments, the medicament container 6200 (and any of the medicament containers and medicament container assemblies described herein) can be a prefilled (or prefillable) syringe, such as those manufactured by Becton Dickinson, Gerresheimer, Ompi Pharma or others. For example, in some embodiments, the medicament container 6200 can be a Becton Dickinson “BD Hypak Physiolis” prefillable syringe containing any of the medicaments described herein. The medicament delivery device 6000 can be configured to deliver any suitable dosage such as, for example, a dose of up to 40 mL of any of the medicaments described herein. In other embodiments, the medicament delivery device 6000 can be configured to inject a dose of up to 2 mL, 3 mL, 4 mL, 5 mL, 6 mL, 7 mL, 8 mL, 9 mL, 10 mL, 20 mL, 30 mL, 50 mL, or more of any of the medicaments described herein.

The container body 6210 can be constructed from glass and can be operably coupled to any suitable needle. For example, in some embodiments, the medicament container 6200 can be coupled to a needle (e.g., via the medicament coupling member 6251 and the needle assembly 6250) having any suitable size, such as a gauge size of 21 gauge, 22 gauge, 23 gauge, 24 gauge, 25 gauge, 26 gauge, 27 gauge, 28 gauge, 29 gauge, 30 gauge, or 31 gauge. Medicament container 6200 can be operably coupled to a needle having any suitable length, such as, for example, a length of about 0.2 inches, about 0.27 inches, about 0.38 inches, about 0.5 inches, about 0.63 inches, about 0.75 inches, or more. In some embodiments, for example, the medicament container 6200 can be coupled to a 29 gauge, needle having a length of approximately 0.5 inches.

In some embodiments, the medicament delivery device 6000 may, as depicted in FIGS. 50, 52, 56-58, 60, 61, 65, and 67, be configured with two medicament containers 6200 (e.g., as a “dual-container injector”). In such an embodiment, one of the medicament containers 6200 may be considered a first medicament container containing a first portion of the medicament and having a first elastomeric member. The first medicament container and the first elastomeric member may define a first medicament container gas chamber that is fluidly coupled to the delivery gas flow path (FP_{B}). The remaining medicament container 6200 may be considered a second medicament container containing a second portion of the medicament and having a second elastomeric member. The second medicament container and the second elastomeric member may define a second medicament container gas chamber that is fluidly coupled to the delivery gas flow path (FP_{B}) in parallel with the first medicament container gas chamber. As such, first medicament container gas chamber and the second medicament container gas chamber are fluidically coupled delivery gas flow path (FP_{B}). Each of the two medicament containers 6200 may contain a portion of the total medicament volume to be delivered. For example, in some embodiments, each medicament container 6200 may contain at least 20 mL of medicament so that at least 40 mL of medicament can be delivered to the patient.

In some embodiments, the medicament container 6200 may have a wet-dry configuration. In such embodiments, the medicament container 6200 may include a first elastomeric member and a second elastomeric member. The first elastomeric member and the second elastomeric member may define, at least in part, a first volume containing a first substance. The second elastomeric member and a portion of the medicament container 6200 may define a second volume that contains a second substance. A movement of the second elastomeric member places the first volume in fluid communication with the second volume to facilitate a mixing of the first substance and the second substance.

Referring to the FIGS. 58-61, as shown, the neck 6213 of the medicament container 6200 is inserted into a receiving portion 6254 at a first end portion 6252 of the medicament coupling member 6251. The medicament coupling member 6251 includes the first end portion 6252 and a second end portion 6253. The medicament coupling member 6251 defines a flow passageway 6261 between the first end portion 6252 and the second end portion 6253. The medicament container 6200 is movably coupled to the first end portion 6252 of the medicament coupling member 6251 in both the first container position and the second container position.

As depicted in FIG. 60, when the medicament container 6200 is in the first container position, the medicament container 6200 is fluidically isolated from the flow passageway 6261. As depicted in FIG. 61, when the medicament container 6200 is in the second container position, the medicament container 6200 is in fluid communication with the flow passageway 6261. For example, as shown, the receiving portion 6254 of the medicament coupling member 6251 includes a penetrator 6259. The penetrator 6259 is oriented to puncture a frangible seal of the medicament container 6200 when the medicament container 6200 transitions from the first container position to the second container position in response to the movement of the flow restriction assembly 6430.

As depicted in FIGS. 58 and 59, the medicament coupling member 6251 is fluidly coupled to the needle assembly 6250 to place the needle 6260 in fluid communication with the flow passageway 6261. For example, a flexible member 6263 is coupled between the second end portion 6253 and the needle assembly 6250. The flexible member 6263 may be a tube, a hose, or other flexible structure that defines a hollow passageway therethrough. Accordingly, the flexible member 6263 extends the flow passageway 6261 from the medicament coupling member 6251 to the needle assembly 6250. As such, when the medicament container 6200 is in the second container position, a portion of the medicament may depart the medicament container 6200, progress through the flow passageway 6261 to the needle assembly 6250, and exit the needle 6260 in response to a longitudinal movement of the elastomeric member 6217.

In some embodiments, the medicament delivery device 6000 includes a flow restriction assembly 6430 that defines the delivery gas flow path (FP_{B}). The flow restriction assembly 6430 includes an assembly body 6429. The assembly body 6429 has a first outer surface 6431 coupled to the medicament container 6200. The assembly body 6429 also has an inner surface 6433 that at least partially defines the delivery gas flow path (FP_{B}). As such, in some embodiments, a flow restriction member 6450 is positioned within the delivery gas flow path (FP_{B}). In other words, the flow restriction member 6450 is supported by the inner surface 6433 of the flow restriction assembly 6430.

The flow restriction member 6450 is configured to regulate a flow of the second portion of the pressurized gas into the medicament container gas chamber via the delivery gas flow path (FP_{B}). In other words, the flow restriction member 6450 is configured to regulate (e.g., limit) the magnitude of the pressure of the second portion of the pressurized gas that acts on the elastomeric member 6217. As shown in FIG. 62, the flow restriction member 6450 includes a sleeve member 6451 and a flow restriction element 6452, with the flow restriction element 6452 being supported within the sleeve member 6451. In some embodiments, the sleeve member 6451 is a metal sleeve. In some embodiments, the metal sleeve is made of stainless steel or brass. In some embodiments, the flow restriction element 6452 is a porous material. In some embodiments, the porous material is sintered porous metal.

For example, in some embodiments, the flow restriction member 6450 is calibrated with nitrogen gas (N₂) at 30 psig (inlet side) to atmosphere (outlet side) at standard temperature and pressure to have a flow rate of between 0.5 to 3 standard cubic centimeter per minute (sccm). In some embodiments, the flow restriction member 6450 is calibrated with nitrogen gas (N₂) at 30 psig (inlet side) to atmosphere (outlet side) at standard temperature and pressure to have a flow rate of between about 0.75 and 1.5 standard cubic centimeter per minute (sccm). In some embodiments, the flow restriction member 6450 is calibrated with nitrogen gas (N₂) at 30 psig (inlet side) to atmosphere (outlet side) at standard temperature and pressure to have a flow rate of about 1 standard cubic centimeter per minute (sccm). As described herein, standard temperature is 60° F. (15.6° C.) and standard pressure is 14.696 psia (101.3 kPa).

In some embodiments, the compressed gas supplied by the energy storage member 6400 is an argon gas and the flow restriction member 6450 has a flow rate rating of about 0.75 and 1.5 sccm based on the nitrogen gas calibration described above. In some embodiments, the compressed gas supplied by the energy storage member 6400 is an argon gas and the flow restriction member 6450 has a flow rate rating of about 1 sccm based on the nitrogen gas calibration described above. In some embodiments, the compressed gas in the energy storage member 6400 has a molecular weight greater than the molecular weight of argon. For example, in some embodiments, the compressed gas supplied by the energy storage member 6400 is R134a (Tetrafluoroethane) and the flow restriction member 6450 has a flow rate rating of about 10 to 100 sccm based on the nitrogen gas calibration described above. In some embodiments, the compressed gas is R134a (Tetrafluoroethane) and the flow restriction member 6450 has a flow rate rating of about 20 to 40 sccm based on the nitrogen gas calibration described above.

In some embodiments, the flow rate of the medicament can be reduced to less than 0.2 mL/sec (or in some embodiments between 0.05 mL/sec and 0.01 mL/see) using gas pressure that is supplied to medicament container gas chamber 6470 via the delivery gas flow path (FP_{B}) and the flow restriction member 6450. The lower injection forces and/or slower delivery (compared with pressures supplied directly from the primary gas chamber 6440) can produce laminar flow of the medicament through the needle, prevent shearing of high molecular weight compounds in the medicament, and/or reduce pain sensed by a patient particularly if the medicament being delivered is very high viscosity (e.g., greater than about 100 centipoise at room temperature). Additionally, the lower injection forces may facilitate the delivery of relatively large volumes of the medicament, such as dosages of greater than 30 mL (e.g., 40 mL).

As depicted in FIGS. 57, 62, and 63, in some embodiments, the flow restriction assembly 6430 includes a restriction member access cover 6453 coupled to the assembly body 6429. The restriction member access cover 6453 may provide access to the inner surface 6433 of the assembly body 6429 and may facilitate the placement of the flow restriction member 6450 during assembly of the medicament delivery device 6000. The restriction member access cover 6453 may be circumscribed by a sealing member to fluidically seal the inner surface 6433 from the second outer surface 6432. Accordingly, a first face of the flow restriction member access cover 6453 that is oriented toward the inner surface 6433 of the assembly body 6429 defines, in conjunction with the assembly body 6429 the delivery gas flow path (FP_{B}). Additionally, a second face of the flow restriction member access cover 6453 is oriented toward the housing cap 6110 and defines a portion of the boundary of the primary gas chamber 6440.

As depicted in FIGS. 58, 59, 62, and 63, in some embodiments, the medicament delivery device 6000 includes the system actuation assembly 6500. The system actuation assembly 6500 includes an input surface 6510 configured to receive a force input. In some embodiments, the input surface 6510 may remain in a depressed position following the force input and may, thus, serve as a visible indication of system actuation. It should be appreciated that the input force may be delivered by a patient, a care provider, a service animal, and/or via automated means.

In some embodiments, the system actuation assembly 6500 is configured to transmit the force input to an inclined plane 6512, a release member (not shown) or other similar structure configured to initiate a release of the pressurized gas from the energy storage member 6400. For example, in some embodiments, the inclined plane 6512 is coupled to the input surface 6510 and is positioned to develop a linear (e.g., longitudinal) motion of the energy storage member 6400 in response to the force input. The linear motion results in the activation (e.g., puncturing) of the energy storage member 6400. In other words, the movement of the inclined plane 6512 may drive the energy storage member onto the penetrator 6575 to release the pressurized gas. Similarly, in some embodiments, the linear motion of the energy storage member 6400 may be driven by an actuation solenoid (not shown) in response to an input signal (e.g., an electronic signal). Further, the linear motion of the energy storage member 6400 may be driven by a compressed spring (not shown) that is released by the received force input.

As depicted in FIGS. 69, 62, and 63, in some embodiments, the medicament delivery device 6000 includes the retraction spring 6380. The retraction spring is configured to exert a force on the needle assembly 6250 along the axis that is orthogonal to a plane defined by the longitudinal axis (A_{LO}) and the lateral axis (A_{LA}) in the direction of the first needle carrier position (e.g., in a direction away from the bottom portion 6104). Following the delivery of the medicament, pressure within the needle actuation gas chamber 6460 may be reduced (e.g., vented) so that the magnitude of the pressure falls below the opposing force exerted on the needle assembly 6250 by the retraction spring 6380. As a result, the needle assembly 6250 moves from the second needle carrier position to the first needle carrier position, thereby positioning the needle 6260 within the housing 6100.

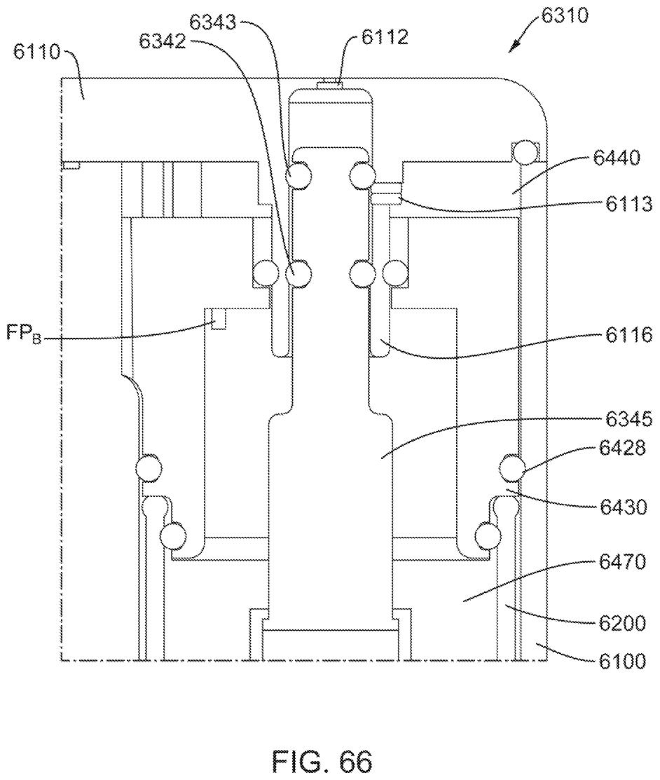

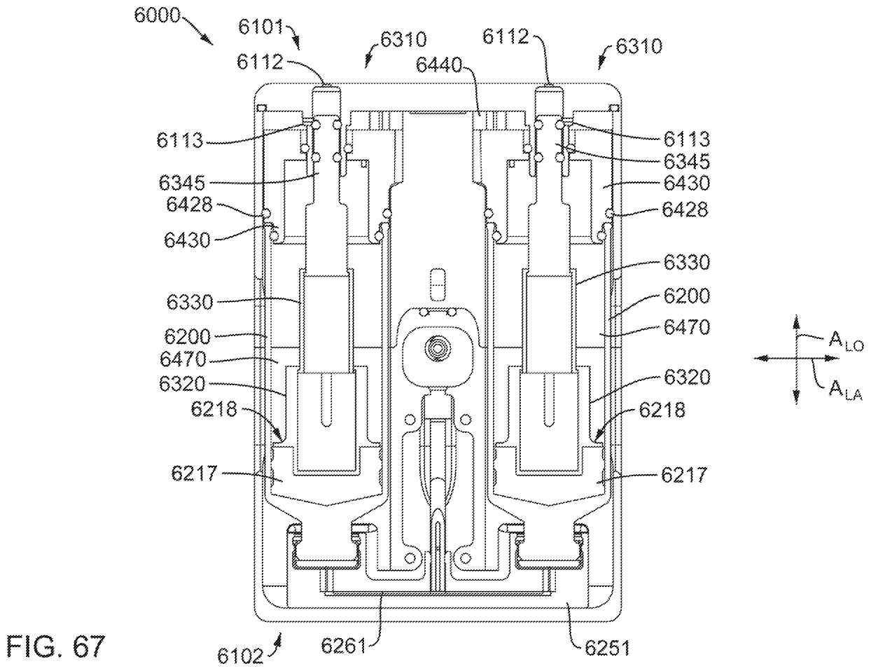

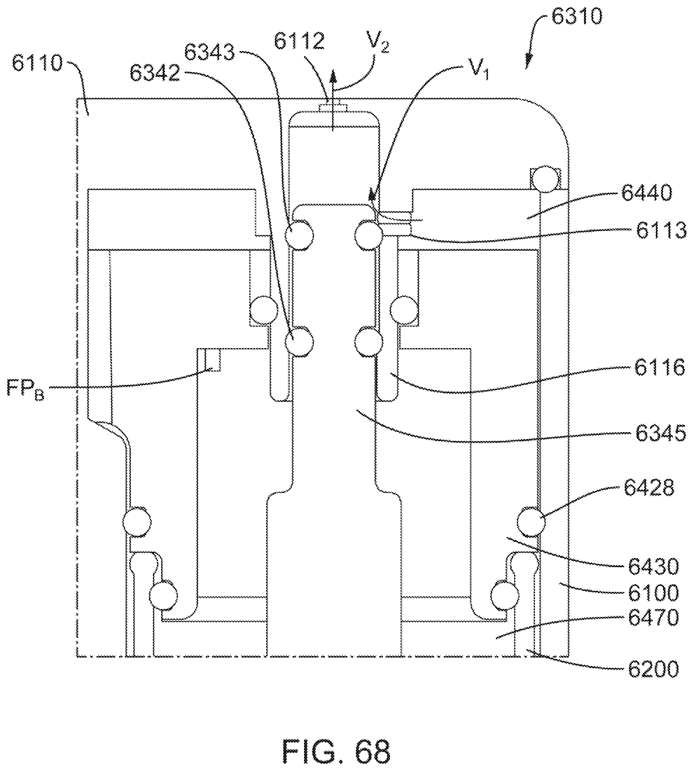

In some embodiments, the medicament delivery device 6000 includes a vent assembly 6310. FIGS. 65 and 67 are cross-sectional views of the medicament delivery device 6000 taken along a plane defined by the longitudinal axis (A_{LO}) and the lateral axis (A_{LA}) that illustrates the vent assembly 6310 according to an embodiment. FIGS. 66 and 68 are enlarged portions of FIGS. 65 and 67, respectively. As depicted, the vent assembly 6310 includes a valve member 6345. The valve member 6345 is, at least partially, positioned within a vent portion 6116 of the housing 6100. For example, in some embodiments, the vent portion 6116 is defined by the housing cap 6110. The vent portion 6116 defines an inlet orifice 6113 that is in fluid communication with the primary gas chamber 6440. The vent portion 6116 also defines the outlet orifice 6112 that is in fluid communication with the exterior volume surrounding the housing 6100. In some embodiments, an outer surface of the vent portion 6116 is circumscribed by a portion of the flow restriction assembly 6430 and a seal member therebetween.

As depicted in FIG. 65-68, in some embodiments, the valve member 6345 includes (e.g., is circumscribed by) a first seal member 6342. The first seal member 6343 establishes a slidable sealed interface between the valve member 6345 and the vent portion 6116 of the housing 6100. The first seal member 6342 is functionally positioned between the inlet orifice 6113 and the medicament container gas chamber 6470. Said another way, the first seal member 6342 is positioned (e.g., longitudinally positioned) between the inlet orifice 6113 and the elastomeric member 6217. The first seal member 6342 is maintained between the inlet orifice 6113 and the medicament container gas chamber 6470 during both the delivery of the medicament and the retraction of the needle 6260. Accordingly, the first seal member 6342 fluidically isolates the medicament container gas chamber 6470 from the inlet orifice 6113 and the outlet orifice 6112 (i.e., from the exterior volume).

In some embodiments, the valve member 6345 is circumscribed by a second seal member 6343 (e.g., an O-ring). The second seal member 6343 establishes a slidable sealed interface between the valve member 6345 and the vent portion 6116 of the housing 6100. The second seal member 6343 is positioned (e.g., longitudinally positioned) between the inlet orifice 6113 and the outlet orifice 6112 when the valve member 6345 is in a first valve position (e.g., a first longitudinal position) as depicted in FIGS. 65 and 66. In the first valve position, the second seal member 6343 fluidically isolates the primary gas chamber 6440 from the exterior volume.

During operation of the medicament delivery device 6000, the valve member 6345 is configured to transition to a second valve position (e.g., a second longitudinal position) as depicted in FIGS. 67 and 68. In the second valve position, the first seal member 6342 remains functionally between the inlet orifice 6113 and the medicament container gas chamber 6470 (e.g., physically between the inlet orifice 6113 and the elastomeric member 6217). In the second valve position, the second seal member 6343 is positioned between the inlet orifice 6113 and the elastomeric member 6217. When the valve member 6345 is in the second valve position, the primary gas chamber 6440 is in fluid communication with the exterior volume via the inlet orifice 6113 and the outlet orifice 6112 (as indicated by arrows V₁ and V₂). The fluid communication between the primary gas chamber 6440 and the exterior volume also places the needle actuation gas chamber 6460 in fluid communication with the exterior volume via the insertion gas flow path (FP_{A}). As the pressure of the exterior volume is less than the pressure of the second portion of the pressurized gas within the needle actuation gas chamber 6460, a flow direction of the insertion gas flow path (FP_{A}) is reversed and the needle actuation gas chamber 6460 is vented through the primary gas chamber 6440 to the exterior volume. The venting of the needle actuation gas chamber 6460 reduces the pressure therein to a magnitude that is less than the opposing force exerted on the needle assembly 6250 by the retraction spring 6380 and the needle 6260 is withdrawn into the housing 6100. The venting of the needle actuation gas chamber 6460 and the corresponding transition of the needle assembly 6250 to the first needle carrier position does not affect the positioning of the medicament container 6200. As such, the medicament container 6200 is maintained in the second container position (e.g., fluidically coupled to the medicament coupling member 6251) following the retraction of the needle 6260.

In some embodiments, the vent assembly 6310 also includes an expandable assembly 6311. The expandable assembly 6311 is configured to transition from a first configuration (e.g., collapsed configuration), such as depicted in FIG. 60, to a second configuration (e.g., an extended or expanded configuration), such as depicted in FIGS. 65 and 67. The expandable assembly 6311 includes a first member 6320, a second (or intermediate) member 6330, and the valve member 6345 (e.g., a third member). In additional embodiments, the expandable assembly 6311 may have any number of additional members. The first member 6320, the second member 6330 and a portion of the valve member 6345 are nested together such that the gas vent assembly 6310 can be transitioned from the collapsed configuration to the expanded configuration, and a series of partially expanded configurations therebetween based on a longitudinal position of the elastomeric member 6217. When the expandable assembly 6311 reaches the expanded configuration (e.g., full extension or a maximal length), the valve member 6345 is transitioned from the first valve position (see e.g., FIG. 66) to the second valve position (see e.g., FIG. 68). In other words, the valve member 6345 is configured to move from the first valve position to the second valve position, when the expandable assembly 6311 transitions from the first configuration to the second configuration, to release the pressurized gas from the primary gas chamber 6440 to the exterior volume surrounding the housing 6100. Stated in a different manner, the length of the expandable assembly 6311 in the expanded configuration is selected to expand to reach the expanded configuration in conjunction with the positioning of the elastomeric member 6217 at a second longitudinal position, such as depicted in FIG. 65. The second longitudinal position depicted in FIG. 65 corresponds to a delivery-complete position of the elastomeric member 6217.

The first member 6320 is configured to matingly engage the elastomeric member 6217. In this manner, movement of the elastomeric member 6217 longitudinally causes a corresponding movement of first member 6320. An end of the first member 6320 that is longitudinally opposite the elastomeric member 6217 includes an inwardly extending lip configured to engage a corresponding outwardly extending lip of the second member 6330 at one end. The opposite end of the second member 6330 includes an inwardly extending lip configured to engage a corresponding outwardly extending lip of the valve member 6345. As the elastomeric member 6217 is moved longitudinally during operation, the first member 6320 is moved longitudinally away from the second member 6330 and the valve member 6345, and the second member 6330 is moved longitudinally away from the valve member 6345. Stated differently, the first member 6320, the second member 6330, and the valve member 6345 transition from a collapsed configuration to an expanded configuration by sliding apart from one another in response to the longitudinal movement of the elastomeric member 6217 that results from the second portion of the pressurized gas within the medicament container gas chamber 6470.

The first member 6320 can be coupled to the elastomeric member 6217 in any suitable manner. For example, as shown, the first surface 6218 of the elastomeric member 6217 receives and/or couples to a protrusion of the first member 6320 of the expandable assembly 6311. In some embodiments, the first member 6320 includes a threaded portion and first surface 6218 includes a corresponding threaded portion to receive the first members 320. In some embodiments, the threaded portion of the first member 6320 is a self-tapping threaded portion. In other embodiments, the first member 6320 can be threadedly coupled to the elastomeric member 6217. In yet other embodiments, the first member 6320 can be bonded to the elastomeric member 6217 via an adhesive, a weld process, or the like.

In some embodiments wherein the medicament delivery device 6000 includes more than one medicament container 6200, the medicament delivery device 6000 may also include more than one vent assembly 6310. For example, in some embodiments, such as depicted in FIGS. 60, 61, 65, and 67, the medicament delivery device 6000 includes a first vent assembly with a first valve member operably coupled to the first elastomeric member. In such embodiments, the medicament delivery device 6000 includes a second vent assembly including a second valve member operably coupled to the second elastomeric member. In such embodiments, the second vent assembly may serve as a redundant vent assembly, ensuring the venting of the medicament delivery device 6000 and retraction of the needle 6260 even if first vent assembly malfunctions or is obstructed in some manner.

To accurately deliver the medicament, the venting of the medicament delivery device 6000 vent assembly 6310 is coordinated with the movement of the elastomeric member 6217 accordingly, in embodiments, the elastomeric member 6217 is configured to move within the medicament container 6200 when a pressure within the medicament container gas chamber 6470 is greater than a first pressure threshold. Said another way, the elastomeric member 6217 remains at a first longitudinal position, as depicted in FIG. 61, until the pressure builds within the medicament container gas chamber 6470. When the magnitude of the pressure of the second portion of pressurized gas within the medicament container gas chamber 6470 is greater than the first pressure threshold, the elastomeric member 6217 is configured to move longitudinally towards the medicament coupling member 6251, thereby introducing a portion of the medicament to the flow passageway 6261. The first pressure threshold may correspond to the starting friction between the elastomeric member 6217 and the container body 6210. As such, the magnitude of the pressure of the second portion of pressurized gas may correspond to a minimum magnitude that overcomes the starting friction.

During the movement of the elastomeric member 6217, the valve member 6345 is maintained in the first valve position and fluidically isolates the primary gas chamber 6440 from the exterior volume. During the movement, the volume of the medicament container gas chamber 6470 continues to expand as the elastomeric member 6217 moves longitudinally away from the flow restriction assembly 6430. Accordingly, the magnitude of the pressure of the second portion of the pressurized gas remains at a level no greater than the minimum magnitude that overcame the starting friction.

The elastomeric member 6217 is configured to continue moving longitudinally within the medicament container 6200 until positioned at a second longitudinal position as depicted in FIG. 65. The second longitudinal position of the elastomeric member 6217 corresponds to a delivery-complete position at which a desired portion of the medicament has been dispensed from the medicament delivery device 6000.

When the elastomeric member 6217 is positioned at the second longitudinal position (e.g., the delivery-complete position), the medicament container gas chamber 6470 has a maximal operational volume. The continued introduction of the second portion of the pressurized gas into the maximal operational volume of the medicament container gas chamber 6470 results in a pressure increase within the medicament container gas chamber 6470. When the pressure within the medicament container gas chamber 6470 is greater than a second pressure threshold, the valve member transitions to the second valve position, as depicted in FIGS. 67 and 68. As such, the second pressure threshold corresponds to the starting friction of the interface between the valve member 6345 and the vent portion 6116.

In embodiments wherein the medicament delivery device 6000 is configured with two medicament containers 6200, the first and second medicament container gas chambers 6470 are in fluid communication via the delivery gas flow path (FP_{B}). The fluid communication facilitates the establishment of both of elastomeric members 6217 at the delivery-complete position within the respective medicament container while the pressure within the respective medicament container gas chambers 6470 remains below the second pressure threshold. The pressure within the respective medicament container gas chambers increasing to a magnitude above the second pressure threshold following the establishment of both of elastomeric members 6217 at the second longitudinal position.

For example, one of the elastomeric members 6217 is stopped at the second longitudinal position while the other elastomeric member 6217 is located between the first longitudinal position and a second longitudinal position. The medicament container gas chamber 6470 corresponding to the elastomeric member that is between the first longitudinal position and the second longitudinal position continues to increase. Due to the fluid communication between the two medicament container gas chambers 6470, the magnitude of the pressure of the second portion of the pressurized gas in both medicament container gas chambers 6470 remains at a level no greater than the minimum magnitude that overcame the starting friction. In other words, the pressure in the first and second medicament container gas chambers 6470 is maintained at a magnitude that is greater than the first pressure threshold and less than the second pressure threshold until each elastomeric member is positioned at the second longitudinal position. Once each elastomeric member 6217 is at the second longitudinal position (e.g., the delivery-complete position), both medicament container gas chamber 6470 has a maximal operational volume and the continued introduction of the second portion of the pressurized gas into the maximal operational volumes results in a pressure increase within each medicament container gas chamber 6470. When the pressure within both medicament container gas chambers 6470 is greater than the second pressure threshold, the valve member transitions to the second valve position, as depicted in FIGS. 67 and 68.

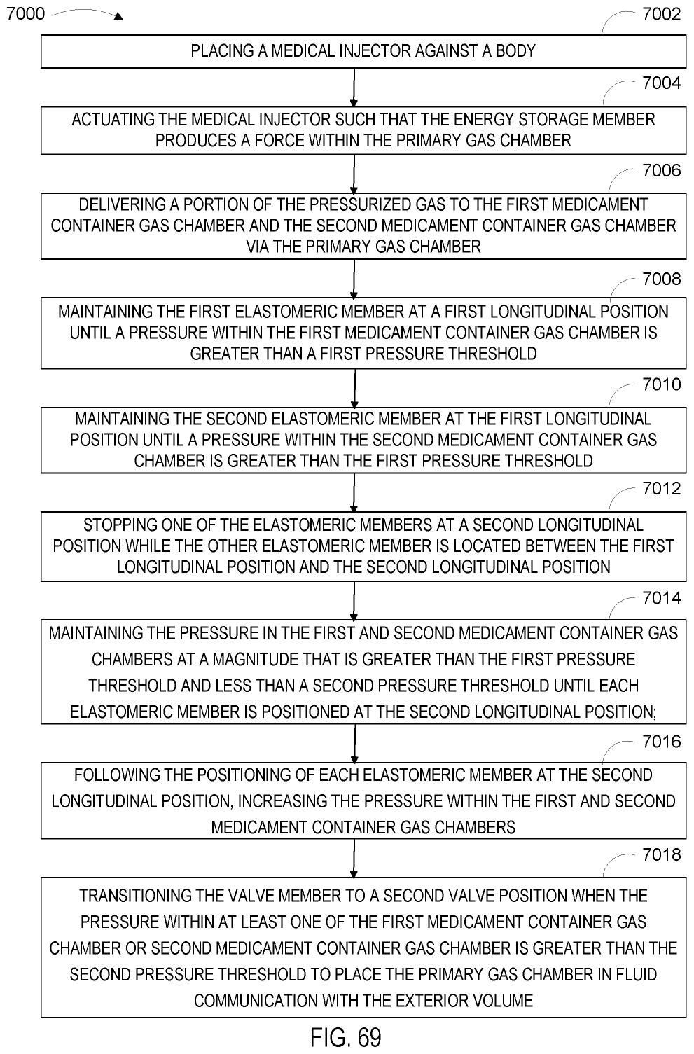

FIG. 69 is a flow chart of a method 7000 of delivering a dose of medicament according to an embodiment. The method 7000 may, in an embodiment, be performed medicament delivery device 6000 as described with reference to FIGS. 50-68. Thus, the method 7000 is described below with reference to medicament delivery device 6000 and in particular the medicament delivery device 6000 when configured with two medicament containers 6200

As illustrated at 7002 in FIG. 69, the method 7000 includes placing a medical injector (e.g., medicament delivery device 6000) against a body of a patient. The medical injector may include any of the elements described above with reference to the medicament delivery device 6000, including, for example, the first and second medicament containers 6200, the housing 6100, the energy storage member 6400, and the vent assembly 6310.

As illustrated at 7004, the method 7000 includes actuating the medical injector such that the energy storage member produces a force within the primary gas chamber. A portion of the pressurized gas is, as indicated at 7006 delivered to the first medicament container gas chamber and the second medicament container gas chamber via the primary gas chamber. As illustrated at 7008, the first elastomeric member is maintained at a first longitudinal position until a pressure within the first medicament container gas chamber is greater than a first pressure threshold. As illustrated at 7010, the second elastomeric member is maintained at the first longitudinal position until a pressure within the second medicament container gas chamber is greater than the first pressure threshold. As previously discussed, when the magnitude of the pressure within the medicament container gas chambers is greater than the first pressure threshold, the respective elastomeric member is configured to move longitudinally within the respective medicament container.

As illustrated at 7012, the method 7000 includes stopping one of the elastomeric members at a second longitudinal position while the other elastomeric member is located between the first longitudinal position and the second longitudinal position. Said another way, manufacturing tolerances may result longitudinal positions of the elastomeric members becoming unsynchronized. As such, one of the elastomeric members may achieve the second longitudinal position before the other elastomeric member. If venting were initiated upon the elastomeric member that first achieving the second longitudinal position, the desired portion of medicament may not be delivered from the other medicament container. For example, the venting may permit the retraction of the needle prior to both of elastomeric members completing a nominal stroke length. As such, as illustrated at 7014, the pressure in the first and second medicament container gas chambers is maintained at a magnitude that is greater than the first pressure threshold and less than a second pressure threshold until each elastomeric member is positioned at the second longitudinal position. The second pressure threshold facilitates the delivery of the dose by ensuring a complete delivery of the required volume of the first and second portions of the medicament.

As depicted at 7016, following the positioning of each elastomeric member at the second longitudinal position, the pressure within the first and second medicament container gas chambers is increased to a magnitude that exceeds the second pressure threshold. Accordingly, as depicted at 7018, the valve member is transitioned to a second position when the pressure within at least one of the first medicament container gas chamber or the second medicament container gas chamber is greater than the second pressure threshold. This transition places the primary gas chamber in fluid communication with the exterior volume.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

For example, any of the devices shown and described herein can include an electronic circuit system to provide user instruction and/or feedback. In some embodiments the electronic circuit system can be integral to the device (e.g., included within the housing, such as the housing 1100, 2100, 3100). In other embodiments, the electronic circuit system can be an external, discrete component that is affixed to the device. In some embodiments, an electronic circuit system can include a contact sensor to determine contact between the bottom portion of an on-body delivery system (e.g., the bottom portion 1104) and the target location. The electronic circuit system can produce a signal to the user to indicate that the device is properly placed for actuation. In some embodiments, an electronic circuit system can include a sensing unit to detect the position of the elastomeric member within the medicament container body and produce an electronic signal associated with at least one of a position, a velocity, or an acceleration of the elastomeric member to detect the rate of medicament delivery. For example, in some embodiments, any of the devices described herein can include any of the electronic circuit systems, modules, and/or sensing units shown and described in Intemational Patent Publication No. 2021/030210, which is incorporated herein by reference in its entirety.

In some embodiments, the medicament delivery device can include a sensor that determines an end of stroke (i.e., plunger position associated with an end of medicament delivery) and causes a switch or other electronic actuator to move the vent valve to release pressurized gas from within the medicament delivery device to retract the needle, as described herein.

In some embodiments, removal of the medicament delivery device from the patient's body can trigger a sensor to activate a latch mechanism to stop the flow of gas temporarily in order to minimize medicament loss until the patient reapplies the medicament delivery device to the body. The patient can resume medicament delivery by disengaging the latch (e.g., actuating a lever or button to disengage the latch mechanism) to allow the flow of gas to resume again and thus the continued delivery of medicament.

In some embodiments, the electronic circuit system can contain an electromechanical mechanism or motor to actuate the actuation mechanism. In some embodiments, the electromechanical mechanism or motor can be included with a mechanical actuation mechanism (which may be similar to or include components from the system actuation assembly 1500 described herein) and the mechanical actuation mechanism may serve as a backup method for operating the medicament delivery device in the event the electromechanical mechanism or motor is inoperative.

In some embodiments, the medicament delivery device can include a sensor, a radio, a memory and a communication module. The radio is configured to electronically communicate with a network or a computing device via a wireless protocol (e.g., Bluetooth©, Wi-Fi, short-range radio, cellular network, satellite, or other communication means). The sensor can monitor a patient's health status, such as a patient's heart rate, blood pressure, or exposure to chemical substances (e.g., fentanyl, radiation, or nerve agents). If the sensor detects that the patient's heart rate or blood pressure is out of a normal range and/or the patient has been exposed to a chemical substance (i.e., detection of an abnormal parameter), the sensor can transmit the detected abnormal parameters to the memory.

In some embodiments, the radio is configured to send a wireless signal, via the radio, to transmit the detected abnormal parameters to a network or computing device to alert a physician and/or health monitoring. In some embodiments, in response to the detected abnormal parameters, the medicament delivery device may provide an audio and/or visual prompt to instruct the patient to operate the delivery device. If the patient does not respond to the audio and/or visual prompt, the electronic circuit system can initiate the electromechanical mechanism or motor to initiate delivery of medicament to the patient. In some embodiments, the medicament delivery device is configured to receive a wireless signal from the physician or health monitoring service to initiate delivery of medicament. In some embodiments, the medicament delivery device is configured to receive an authentication signal and initiate delivery of medicament after verifying that the authentication signal is valid to prevent unauthorized access or control of the medicament delivery device.

In some embodiments, the medicament delivery device can include one or more sensors to monitor a patient's health status such as heart rate, blood pressure, or exposure to chemical substances (e.g., fentanyl, radiation, or nerve agents). If the one or more sensors detect abnormal parameters, the medicament delivery device can activate to deliver medicament without further input or action from the patient. For example, if the sensors detect that the patient is bleeding (e.g., hemorrhage), the medicament delivery device can deliver an anti-hemorrhage medicament to the patient once the hemorrhage condition is detected. In some embodiments, the medicament delivery device includes an override or termination input for the patient to abort or terminate medicament delivery.

In some embodiments, an auxiliary sensor detecting physiological parameters of the patient can be provided separate from the medicament delivery device. The auxiliary sensor is configured to send a wireless signal to the medicament delivery device and/or to the health monitoring service in response to abnormal parameters being detected. The delivery device is configured to receive a wireless signal from the auxiliary sensor to produce an audio and/or visual prompt, or receive a wireless signal from the auxiliary sensor to initiate delivery of medicament. In some embodiments, the medicament delivery device is configured to receive a wireless signal from the health monitoring service to produce an audio and/or visual prompt, or receive a wireless signal from the health monitoring service to initiate delivery of medicament.

Certain aspects of the electronics control system and the medicament delivery device with connected health aspects can be similar to or substantially the same to the medical injectors described in the in International Application No. PCT/US2018/013855 entitled, “MEDICAMENT DELIVERY DEVICES WITH WIRELESS CONNECTIVITY AND EVENT DETECTION,” filed on Jan. 16, 2018, U.S. patent application Ser. No. 15/872,162 (now U.S. Pat. No. 10,332,623) entitled, “MEDICAMENT DELIVERY DEVICES WITH WIRELESS CONNECTIVITY AND EVENT DETECTION,” filed on Jan. 16, 2018, U.S. patent application Ser. No. 16/421,639 (now U.S. Pat. No. 10,937,537) entitled, “MEDICAMENT DELIVERY DEVICES WITH WIRELESS CONNECTIVITY AND EVENT DETECTION,” filed on May 24, 2019, and U.S. patent application Ser. No. 17/186,896 entitled, “MEDICAMENT DELIVERY DEVICES WITH WIRELESS CONNECTIVITY AND EVENT DETECTION,” filed on Feb. 26, 2021, each of which is incorporated herein by reference in its entirety.

For example, any of the elastomeric members described herein can be constructed from any suitable material or combination of different materials. For example, in some embodiments, at least a portion of any of the elastomeric members described herein (e.g., the elastomeric members 1217, 2217, 3217) can be coated. Such coatings can include, for example, polydimethylsiloxane. In some embodiments, at least a portion of any of the elastomeric members described herein can be coated with polydimethylsiloxane in an amount of between approximately 0.02 mg/cm² and approximately 0.80 mg/cm².

Any of the medicament container assemblies described herein can have any suitable size (e.g., length and/or diameter) and can contain any suitable volume of the medicament. In some embodiments, any of the medicament container assemblies described herein (including the medicament container assemblies 1200, 2200, 3200,) can be a prefilled (or prefillable) syringe, such as those manufactured by Becton Dickinson, Gerresheimer, Ompi Pharma or others. For example, in some embodiments, the medicament container assembly 1200 (and any of the medicament container assemblies described herein) can be a Becton Dickinson “BD Hypak Physiolis” prefillable syringe containing any of the medicaments described herein. Moreover, any of the medicament delivery devices and/or medical injectors described herein can be configured to inject any suitable dosage such as, for example, a dose of up to 1 mL of any of the medicaments described herein. In other embodiments, any of the medicament delivery devices and/or medical injectors described herein can be configured to inject a dose of up to 2 mL, 3 mL, 4 mL, 5 mL, 6 mL, 7 mL, 8 mL, 9 mL, 10 mL, or more of any of the medicaments described herein.

Any of the container bodies described herein can be constructed from glass, and can be fitted and/or coupled to any suitable needle via a needle assembly (including needle assemblies 1250, 2250, 3250). For example, in some embodiments, any of the container bodies described herein (including the container bodies 1210, 2210, 3210) can be coupled to a needle having any suitable size. Any of the medicament container assemblies and/or prefilled syringes described herein can be coupled to a needle having a gauge size of 21 gauge, 22 gauge, 23 gauge, 24 gauge, 25 gauge, 26 gauge, 27 gauge, 28 gauge, 29 gauge, 30 gauge, or 31 gauge. Any of the medicament container assemblies and/or prefilled syringes described herein can be coupled to a needle having any suitable length, such as, for example, a length of about 0.2 inches, about 0.27 inches, about 0.38 inches, about 0.5 inches, about 0.63 inches, about 0.75 inches, or more. In some embodiments, any of the medicament containers and/or prefilled syringes described herein can be coupled to a 29 gauge, needle having a length of approximately 0.5 inches. Moreover, any of the medicament containers and/or prefilled syringes described herein can include a staked needle at the distal end thereof.

Although the medicament injectors shown and described above include a delivery mechanism (e.g., 1300, 2300, 3300) including the release of a pressurized gas, in other embodiments, a medicament delivery device can include any suitable method of delivery of a medicament disposed within. For example, in some embodiments, any of the devices described herein can include a mechanical energy storage (e.g. spring, gears, racks, pinions, pulleys, or the like) member, rather than a compressed gas container. In other embodiments, any of the devices described herein can include any other suitable energy storage member (e.g., magnetic, electrical, propellant based, chemical reaction based, or the like).

While the medical injectors herein are described as being “pistonless” gas-powered auto-injectors, in other embodiments, any of the medical injectors can include any suitable energy storage member configured to produce a force directly on a medicament container and/or a carrier (as described, for example, in the ′849 patent). For example, in some embodiments, a medical injector can include one or more bias members, springs, and/or any other suitable mechanical drives (as described above) configured to exert a force on one or more medicament containers. By way of example, a medical injector can include a first spring configured to produce a force on a first medicament container and a second spring configured to produce a force, substantially equal to the force produced by the first spring, on a second medicament container. Moreover, the first spring and the second spring can be actuated substantially concurrently and/or via the same actuation event such that the first spring and second spring move the first medicament container and the second medicament container substantially concurrently.

Although some of the “dual container” injectors have been described above as being moved in response to a force produced by a single energy storage and/or the same type of energy storage member, in other embodiments, a medicament container can include any suitable combination of energy storage members. For example, in some embodiments, a medical injector can include a first compressed gas container configured to release a volume of compressed gas to move a first medicament container relative to a housing, and a second compressed gas container configured to release a volume of compressed gas to move a second medicament container relative to the housing. In other embodiments, a medical injector can include a compressed gas container configured to release a volume of compressed gas and a spring configured to transition from a first configuration to a second configuration. In such embodiments, for example, the first medicament container can be moved in response to a force associated with the expansion of the compressed gas while the second medicament container can be moved in response to a force associated with the transitioning of the spring from the first configuration to the second configuration (or vice versa). In other embodiments, the forces produced by the expansion of the compressed gas and the transitioning of the spring can be collectively exerted on both the first medicament container and the second medicament container.

Although the embodiments have been particularly described above as moving the insertion member, the medicament containers, and/or the plunger in a substantially concurrent injection event, in other embodiments, a medical injector can be configured for a “staged” (or sequential) injection event. For example, in some embodiments, a medical injector can include a first energy storage member (such as any of those described herein) configured to exert a force on the insertion member and a first medicament container, and a second energy storage member (similar to or different from the first energy storage member) configured to exert a force on a second medicament container. In such embodiments, actuation of the medical injector can result in the first energy storage member exerting the force on the first medicament container to initiate a first injection event (i.e., delivery of a first medicament from the first medicament container), while the second energy storage member remains in a configuration associated with a greater potential energy (e.g., unactuated or the like). After a predetermined time after the actuation of the medical injector, the second energy storage member can exert the force on the second medicament container to initiate a second injection event (i.e., delivery of a second medicament from the second medicament container). By way of example, a medical injector can include a first compressed gas storage container configured to release a volume of compressed gas to initiate an injection event associated with a first medicament container and a second compressed gas storage container configured to release a volume of compressed gas to initiate an injection event associated with a second medicament container. In such embodiments, actuation of the medical injector can result in (1) the first gas storage container being punctured (or actuated) at a first time to initiate the injection event associated with the first medicament container and (2) the second gas storage container being punctured (or actuated) at a second time, after the first time, to initiate the injection event associated with the second medicament container.

In other embodiments, the second energy storage member can exert the force on the second medicament container in response to a second actuation event. For example, a medical injector can include an actuator (e.g., a base or the like) configured to be actuated (e.g., moved) a first amount and a second amount after the first amount. By way of example, a medical injector can include a base actuator configured to be moved a first distance to actuate a first energy storage and a second distance to actuate a second energy storage member. In such embodiments, the movement of the base actuator can be substantially continuous. That is to say, the base actuator can be moved the second distance in a single continuous motion and, while moving through a distance substantially equal to the first distance, can trigger an actuation of the first energy storage member. In other embodiments, the movement of the base actuator the first amount can be a discrete operation and the movement of the base actuator the second amount can be a discrete operation.

In still other embodiments, the medical injector can include a first actuator configured to actuate the first energy storage member and a second actuator configured to actuate the second energy storage member. For example, in some embodiments, a user can manipulate the medical injector to actuate the first actuator (e.g., by moving a base or the like, as described above), which in turn actuates the first energy storage member. After the first actuator is actuated, the user can manipulate the second actuator, which in turn actuates the second energy storage member. In some embodiments, the first actuator can be configured to actuate the second actuator after an actuation event. In other embodiments, the second actuator can be discretely and/or otherwise independently actuated by the user. For example, in some embodiments, a medical injector can include a first actuator disposed on or at a first end portion of the medical injector and can include a second actuator disposed on or at a second end portion of the medical injector opposite the first end portion. In some such embodiments, the first actuator and the second actuator can be actuated and/or moved in response to forces exerted in the same direction while the medical injector is in a substantially constant orientation.

Although the medicament containers are described above as being actuated (either concurrently or independently) to perform an injection event of a medicament directly into a patient, in other embodiments, an injection event of a first medicament container or a second medicament container need not result in direct injection of the medicament into the patient. For example, in some embodiments, a medical injector can include a first medicament container including a needle assembly coupled to a distal end portion of the first medicament container, and a second medicament container in fluid communication with the first medicament container. In such embodiments, actuation of the medical injector can result in, for example, an injection event in which the second medicament container injects a volume of medicament contained therein into the first medicament container. Moreover, in a substantially simultaneous process, the needle assembly coupled to the first medicament container can be moved to insert the needle into the patient. This arrangement can be such that a complete insertion of the needle into the patient substantially corresponds with and/or occurs substantially at the same time as an injection of the medicament from the second medicament container into the first medicament container.

Although particular injection events, mechanisms, devices, and/or components have been described herein, it is to be understood that they have been presented by way of example and not limitation. That is to say, an auto-injector can include more than one medicament container and can be configured to deliver at least one dose of a medicament to a patient in response any suitable actuation event and/or the like.

Any of the devices and/or medicament containers shown and described herein can be constructed from any suitable material. Such materials include glass, plastic (including thermoplastics such as cyclic olefin copolymers), or any other material used in the manufacture of prefilled syringes containing medications.

Any of the devices and/or medicament containers shown and described herein can contain and/or deliver a wide array of large or macromolecular injectables that include carbohydrate-derived formulations, lipids, nucleic acids, nucleic acids, hyaluronidase, proteins/peptides (e.g. monoclonal antibodies), anti-hemorrhagic agents, hemostatic agents (e.g., tranexamic acid, , ω-aminocaproic acid, anti-inhibitor coagulant complex-heat treated, anti-hemophilic factor, factor IX, carbazochrome, fibrinogen concentrate, oprelvekin and phylloquinone), local acting agents (e.g., cellulose, collagen, gelatin, thrombin and thrombin combination products), and other biotechnologically-derived medicaments. For example, anti-tumor necrosis factor agents such as infliximab, etanercept, adalimumab, golimumab, natalizumab, vedolizumab, and certolizumab can be administered using the described auto-injector heroin, Other macromolecular injectable medications that can be administered using the device and/or medicament containers shown and described herein include viscous medicaments that target pro-inflammatory cytokines (e.g. IL-1, IL-2, IL-4, IL-5, IL-6, IL-12, IL-13, IL-23, IL-17, IL-21, IL-23A, and associated receptors) including dupilumab, daratumumab, sarilumab, mepolizumab, benralizumab, reslizumab, lebrikizumab, ustekinumab, anrunkinzumab, bertilimumab, tralokinumab, and risankizumab. Large anti-adhesion molecules to treat a variety of diseases may be administered using the device and/or medicament containers shown and described herein including etrolizumab and vatelizumab. Still other large and viscous monoclonal antibodies that may be administered using the device and/or medicament containers shown and described herein include tezepelumab, anifrolumab, omalizumab, and proprotein convertase subtilisin kexin type 9 (PCSK9) inhibitors including alirocumab and evolocumab.

Any of the devices and/or medicament containers shown and described herein can include any suitable medicament or therapeutic agent. In some embodiments, the medicament contained within any of the medicament containers shown herein can be a vaccine, such as, for example, an influenza vaccine, a hepatitis vaccine, a haemophilus influenza Type B (HiB) vaccine, a measles vaccine, a mumps vaccine, a rubella vaccine, or combination vaccine (e.g. measles, mumps and rubella, quadrivalent, or hexavalent vaccines), a polio vaccine, a human papilloma virus (HPV) vaccine, a tetanus vaccine, a diphtheria vaccine, a pertussis vaccine, a bubonic plague vaccine, a yellow fever vaccine, a cholera vaccine, a malaria vaccine, a smallpox vaccine, a pneumococcal vaccine, a rotavirus vaccine, a varicella vaccine, a dengue fever vaccine, a rabies vaccine and/or a meningococcus vaccine. In other embodiments, the medicament contained within any of the medicament containers shown herein can be a catecholamine, such as epinephrine. In other embodiments, the medicament contained within any of the medicament containers shown herein can be an opioid receptor antagonist, such as naloxone, including any of the naloxone formulations described in U.S. Pat. No. 8,627,816, entitled “Medicament Delivery Device for Administration of Opioid Antagonists Including Formulation for Naloxone,” filed on Feb. 28, 2011. In yet other embodiments, the medicament contained within any of the medicament containers shown herein can include peptide hormones such as insulin and glucagon; human growth hormone (HGH); sumatriptan; a corticosteroid such as dexamethasone; ondansetron; an opioid agonist receptor modulators such as fentanyl; a partial agonist opioid receptor modulators such as buprenorphine; a mixed agonist/antagonist opioid receptor modulator such as nalbuphine; a benzodiazepine such as diazepam, midazolam or lorazepam; erythropoiesis-stimulating agents (ESA) such as darbepoetin alfa; immunoglobulins including dual-variable domain immunoglobulins; monoclonal antibodies such as denosumab, romosozumab, adalimumab, proprotein convertase subtilisin kexin type 9 (PCSK9) inhibitors including alirocumab, anti-interleukins such as anti-IL4R or anti-IL6R antibodies; interferons; anti-tumor necrosis factor (anti-TNF) agents such as etanercept; radiation exposure treatments; GM-CSF (Sargramostil) drugs and biologics; recombinant human granulocyte colony-stimulating factor (GCSF) such as pegfilgrastim; and other therapies suitable for injection in mammals. In yet other embodiments, the medicament contained within any of the medicament containers shown herein can be a placebo substance (i.e., a substance with no active ingredients), such as water.

The medicament containers and/or medicament delivery devices disclosed herein can contain any suitable amount of any medicament. For example, in some embodiments, a medicament delivery device as shown herein can be a single-dose device containing an amount medicament to be delivered of approximately 0.4 mg, 0.8 mg, 1 mg, 1.6 mg or 2 mg. As described above, the fill volume can be such that the ratio of the delivery volume to the fill volume is any suitable value (e.g., 0.4, 0.6 or the like). In some embodiments, an electronic circuit system can include “configuration switch” that, when actuated during the assembly of the delivery device, can select an electronic output corresponding to the dose contained within the medicament container.

Any of the medicament containers described herein can include any suitable elastomeric member and/or plunger. For example, an elastomeric member can be formulated to be compatible with the medicament contained within a medicament container. Moreover, a medicament container can include any number of elastomeric members. For example, in some embodiments, a medicament container can include a dry portion of a medicament and a fluid portion of the medicament, configured to be mixed before injection. The piston portion of the medicament delivery mechanism can be configured to engage multiple elastomeric members associated with the portions of the medicament. In this manner, multiple elastomeric members can be engaged to mix the dry portion with the fluid portion of the medicament before the completion of an injection event. In some embodiments, for example, any of the devices shown and described herein can include a mixing actuator similar to the mixing actuators shown and described in U.S. Pat. No. 9,173,999, entitled “Devices and Methods for Delivering Medicaments from a Multi-Chamber Container,” filed Jan. 25, 2012, which is incorporated herein by reference in its entirety.

Although the injectors described herein have been shown and described as including mechanisms for needle retraction, in other embodiments any of the injectors shown and described herein can include a needle shield that extends distally after the injection to cover the exposed needle. Such a design may be used, for example, in a “pistonless” design as discussed above.

In some embodiments, the electronic circuit system of the types described herein can be used in either an actual medicament delivery device or a simulated medicament delivery device. A simulated medicament delivery device can, for example, correspond to an actual medicament delivery device and can, for example, facilitate training a user in the operation of the corresponding actual medicament delivery device.

The simulated medicament delivery device can simulate the actual medicament delivery device in any number of ways. For example, in some embodiments, the simulated medicament delivery device can have a shape corresponding to a shape of the actual medicament delivery device, a size corresponding to a size of the actual medicament delivery device and/or a weight corresponding to a weight of the actual medicament delivery device. Moreover, in some embodiments, the simulated medicament delivery device can include components that correspond to the components of the actual medicament delivery device. In this manner, the simulated medicament delivery device can simulate the look, feel and sounds of the actual medicament delivery device. For example, in some embodiments, the simulated medicament delivery device can include external components (e.g., a housing, a needle guard, a sterile cover, a safety lock or the like) that correspond to external components of the actual medicament delivery device. In some embodiments, the simulated medicament delivery device can include internal components (e.g., an actuation mechanism, a compressed gas source, a medicament container or the like) that correspond to internal components of the actual medicament delivery device.

In some embodiments, however, the simulated medicament delivery device can be devoid of a medicament and/or those components that cause the medicament to be delivered (e.g., a needle, a nozzle or the like). In this manner, the simulated medicament delivery device can be used to train a user in the use of the actual medicament delivery device without exposing the user to a needle and/or a medicament. Moreover, the simulated medicament delivery device can have features to identify it as a training device to prevent a user from mistakenly believing that the simulated medicament delivery device can be used to deliver a medicament. For example, in some embodiments, the simulated medicament delivery device can be of a different color than a corresponding actual medicament delivery device. Similarly, in some embodiments, the simulated medicament delivery device can include a label clearly identifying it as a training device.

Although various embodiments have been described as having particular features and/or combinations of components, other embodiments are possible having a combination of any features and/or components from any of embodiments where appropriate. For example, any of the devices shown and described herein can include an electronic circuit system as described herein.

1. An apparatus, comprising:
a housing defining a primary gas chamber and an insertion gas flow path;
a medicament container within the housing, the medicament container containing a medicament and including an elastomeric member that seals the medicament within the medicament container, the medicament container and the elastomeric member defining a medicament container gas chamber;
a needle assembly including a needle carrier and a needle coupled to the needle carrier, the needle carrier defining a portion of a boundary of a needle actuation gas chamber, the needle carrier configured to move within the housing between a first needle carrier position and a second needle carrier position, the needle being within the housing when the needle carrier is in the first needle carrier position, the needle being outside of the housing when the needle carrier is in the second needle carrier position;
a flow restriction assembly disposed within the housing, the flow restriction assembly defining a portion of a boundary of the primary gas chamber and a delivery gas flow path, the flow restriction assembly configured to move within the housing to move the medicament container between a first container position and a second container position, the needle carrier being in fluid communication with the medicament container when the medicament container is in the second container position; and
an energy storage member configured to produce a pressurized gas when the energy storage member is actuated, the pressurized gas flowing into the primary gas chamber to move the medicament container from the first container position to the second container position, a first portion of the pressurized gas flowing within the insertion gas flow path and into the needle actuation gas chamber to move the needle carrier from the first needle carrier position to the second needle carrier position, a second portion of the pressurized gas flowing through the delivery gas flow path and into the medicament container gas chamber to move the elastomeric member within the medicament container.

2. The apparatus of claim 1, wherein the first portion of the pressurized gas has a pressure that is greater than a pressure of the second portion of the pressurized gas.

3. The apparatus of claim 1, further comprising:
a medicament coupling member having a first end portion and a second end portion and defining a flow passageway between the first end portion and the second end portion, the medicament container movably coupled to the first end portion of the medicament coupling member between the first container position and the second container position, wherein when the medicament container is in the first container position the medicament container is fluidically isolated from the flow passageway, when the medicament container is in the second container position the medicament container is in fluid communication with the flow passageway; and
wherein the needle carrier is coupled to the second end portion of the medicament coupling member to place the needle in fluid communication with the flow passageway.

4. The apparatus of claim 3, wherein:
the needle assembly is coupled to the medicament coupling member via a flexible member positioned between the second end portion and the needle assembly.

5. The apparatus of claim 1, wherein:
the housing and the flow restriction assembly define an intermediate gas flow path disposed downstream of the primary gas chamber and fluidly coupling the primary gas chamber to the insertion gas flow path and the delivery gas flow path.

6. The apparatus of claim 1, wherein:
the insertion gas flow path is configured in a parallel arrangement with the delivery gas flow path.

7. The apparatus of claim 1, wherein the flow restriction assembly includes:
an assembly body having a first outer surface coupled to the medicament container and a second outer surface that forms the portion of the boundary of the primary gas chamber, the assembly body having an inner surface at least partially defining the delivery gas flow path;
a flow restriction member within the delivery gas flow path, the flow restriction member being configured to regulate a flow of the second portion of the pressurized gas into the medicament container gas chamber that acts on the elastomeric member; and
a restriction member access cover coupled to the assembly body, the restriction member access cover and the assembly body defining the delivery gas flow path.

8. The apparatus of claim 1, further comprising:
a retraction spring configured to move the needle assembly towards the first needle carrier position.

9. The apparatus of claim 1, further comprising:
an expandable assembly being configured to transition from a first configuration to a second configuration when the elastomeric member moves within the medicament container, the expandable assembly including:
a first member coupled to the elastomeric member,
a second member coupled to the first member, and
a valve member coupled to the second member, the valve member configured to move from a first valve position to a second valve position when the expandable assembly transitions from the first configuration to the second configuration to release the pressurized gas from the primary gas chamber to an exterior volume surrounding the housing.

10. The apparatus of claim 9, wherein:
the housing defines a vent portion circumscribing the valve member, the vent portion defining an inlet orifice in fluid communication with the primary gas chamber and an outlet orifice in fluid communication with the exterior volume;
the valve member includes a first seal member positioned between the inlet orifice and the medicament container gas chamber to fluidically isolate the medicament container gas chamber from the inlet orifice and the outlet orifice;
the valve member includes a second seal member positioned between the inlet orifice and the outlet orifice when the valve member is in the first valve position to fluidically isolate the primary gas chamber from the exterior volume; and
the second seal member being positioned between the inlet orifice and the medicament container gas chamber when the valve member is in the second valve position to place the primary gas chamber in fluid communication with the exterior volume.

11. The apparatus of claim 1, further comprising:
a system actuation assembly, the system actuation assembly including:
an input surface configured to receive a force input, and
an inclined plane coupled to the input surface, the inclined plane being positioned to develop a linear motion of the energy storage member in response to the force input.

12. The apparatus of claim 1, further comprising:
an actuation solenoid positioned to develop a linear motion of the energy storage member in response to an input signal.

13. The apparatus of claim 1, wherein the medicament container is a first medicament container containing a first portion of the medicament, the apparatus further comprising:
a second medicament container containing a second portion of the medicament.

14. The apparatus of claim 1, wherein the elastomeric member is a first elastomeric member, the medicament container further including:
a second elastomeric member, the first elastomeric member and the second elastomeric member defining, at least in part, a first volume containing a first substance, the second elastomeric member and a portion of the medicament container defining a second volume containing a second substance, a movement of the second elastomeric member placing the first volume in fluid communication with the second volume.

15. An apparatus, comprising:
a housing defining a primary gas chamber and an insertion gas flow path;
a medicament container within the housing, the medicament container containing a medicament and including an elastomeric member that seals the medicament within the medicament container, the medicament container and the elastomeric member defining a medicament container gas chamber;
a needle assembly including a needle carrier and a needle coupled to the needle carrier, the needle carrier coupled to medicament container by a coupling member that selectively places the needle in fluid communication with the medicament container, the needle carrier defining a portion of a boundary of a needle actuation gas chamber, the needle carrier configured to move within the housing between a first needle carrier position and a second needle carrier position, the needle being within the housing when the needle carrier is in the first needle carrier position, the needle being outside of the housing when the needle is in the second needle carrier position;
a flow restriction assembly disposed with the housing, the flow restriction assembly defining a portion of a boundary of the primary gas chamber and a delivery gas flow path, the flow restriction assembly configured to move within the housing to move the medicament container between a first container position and a second container position; and
an energy storage member configured to produce a pressurized gas when the energy storage member is actuated, the pressurized gas flowing into the primary gas chamber to move the medicament container from the first container position to the second container position, a first portion of the pressurized gas flowing within the insertion gas flow path and into the needle actuation gas chamber to move the needle carrier from the first needle carrier position to the second needle carrier position, a second portion of the pressurized gas flowing through the delivery gas flow path and into the medicament container gas chamber to move the elastomeric member within the medicament container.

16.-46. (canceled)
